SEQUENCE LISTING (133 sequences)

SEQ ID NO: 1 - protein, 119 aa, Artificial Sequence
  SIRP peptide variant (general formula I) peptide
  PEPTIDE (31)..(31): Xaa is selected from Leu or Trp
  PEPTIDE (51)..(51): Xaa is selected from Met, Val, Phe, Ile or Leu
  PEPTIDE (52)..(52): Xaa is selected from Gln, Ser or Thr
  PEPTIDE (54)..(54): Xaa is selected from Glu, Thr or Arg
  PEPTIDE (56)..(56): Xaa is selected from His or Arg
  PEPTIDE (70)..(70): Xaa is selected from Asp, Gln or Glu
  PEPTIDE (72)..(72): Xaa is selected from Ile, Val, Met, Arg or Lys
  PEPTIDE (112)..(112): Xaa is selected from Met or Val
EEELQMIQPEKLLLVTVGETATLHCTVTSLXPVGPVLWFRGVGPGRELIYXXGXGXFPRVTTVSDLTKRXNXDFSIRISSITPADVGTYYCVKFRKGSPEDVEFKSGPGTEXALGAKPS

SEQ ID NO: 2 - protein, 119 aa, Artificial Sequence
  SIRP peptide variant (general formula II) peptide
  PEPTIDE (31)..(31): Xaa is selected from Leu or Trp
  PEPTIDE (52)..(52): Xaa is selected from Gln, Ser or Thr
  PEPTIDE (54)..(54): Xaa is selected from Glu, Thr or Arg
  PEPTIDE (56)..(56): Xaa is selected from His or Arg
  PEPTIDE (70)..(70): Xaa is selected from Asp, Gln or Glu
  PEPTIDE (72)..(72): Xaa is selected from Ile, Val, Met, Arg or Lys
  PEPTIDE (112)..(112): Xaa is selected from Met or Val
EEELQMIQPEKLLLVTVGETATLHCTVTSLXPVGPVLWFRGVGPGRELIYRXGXGXFPRVTTVSDLTKRXNXDFSIRISSITPADVGTYYCVKFRKGSPEDVEFKSGPGTEXALGAKPS

SEQ ID NO: 3 - protein, 129 aa, Artificial Sequence
  CD47-ECD-His peptide
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNEHHHHHH

SEQ ID NO: 4 - protein, 355 aa, Artificial Sequence
  CD47-ECD-Fc peptide
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNEEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 350 aa, Artificial Sequence
  SIRP-Fc peptide
EEELQIIQPDKSVSVAAGESAILHCTITSLFPVGPIQWFRGAGPARVLIYNQRQGPFPRVTTVSETTKRENMDFSISISNITPADAGTYYCIKFRKGSPDTEFKSGAGTELSVRAKPSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 120 aa, Artificial Sequence
  9-2 H5 heavy chain variable region domain
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSS

SEQ ID NO: 7 - protein, 113 aa, Artificial Sequence
  9-2 L11 light chain variable region domain
DIVMTQSPDSLAVSLGERATINCKSSQSLFYHSNQKHSLAWYQQKPGQPPKLLIYGASTRESGVPDRFSGSGSGTDFTLTISSLQAEDVAVYYCQQYYGYPYTFGGGTKVEIK

SEQ ID NO: 8 - protein, 119 aa, Artificial Sequence
  24D5 H12 heavy chain variable region domain
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSS

SEQ ID NO: 9 - protein, 111 aa, Artificial Sequence
  24D5 L61 light chain variable region domain
DIVLTQSPASLAVSPGQRATITCRASESVSIHGTHLMHWYQQKPGQPPKLLIYAASNLESGVPARFSGSGSGTDFTLTINPVEAEDTANYYCQQSFEDPLTFGQGTKLEIK

SEQ ID NO: 10 - protein, 327 aa, Artificial Sequence
  IgG4-S228P heavy chain constant region domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 330 aa, Artificial Sequence [sequence omitted]

SEQ ID NO: 12 - protein, 107 aa, Artificial Sequence [sequence omitted]

SEQ ID NO: 13 - protein, 446 aa, Artificial Sequence
  h1830 heavy chain peptide
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 14 - protein, 220 aa, Artificial Sequence
  h1830 light chain peptide
DIVMTQSPDSLAVSLGERATINCKSSQSLFYHSNQKHSLAWYQQKPGQPPKLLIYGASTRESGVPDRFSGSGSGTDFTLTISSLQAEDVAVYYCQQYYGYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 450 aa, Artificial Sequence
  h1830G1 heavy chain peptide
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 445 aa, Artificial Sequence
  h1831 heavy chain peptide
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 17 - protein, 218 aa, Artificial Sequence
  h1831 light chain peptide
DIVLTQSPASLAVSPGQRATITCRASESVSIHGTHLMHWYQQKPGQPPKLLIYAASNLESGVPARFSGSGSGTDFTLTINPVEAEDTANYYCQQSFEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 449 aa, Artificial Sequence
  h1830G1 heavy chain peptide
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 357 bp, Homo sapiens
gaggaggagctacagatgattcagcctgagaagctcctgttggtcacagttggaaagacagccactctgcactgcactgtgacctccctgcttcccgtgggacccgtcctgtggttcagaggagttggaccaggccgggaattaatctacaatcaaaaagaaggccacttccccagggtaacaacagtttcagacctcacaaagagaaacaacatggacttttccatccgcatcagtagcatcaccccagcagatgtcggcacatactactgtgtgaagtttcgaaaagggagccctgagaacgtggagtttaagtctggaccaggcactgagatggctttgggtgccaaaccctct

SEQ ID NO: 20 - protein, 119 aa, Artificial Sequence
  Wild-type SIRP peptide
EEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPS

SEQ ID NO: 21 - protein, 119 aa, Artificial Sequence
  S58 peptide
EEELQMIQPEKLLLVTVGETATLHCTVTSLLPVGPVLWFRGVGPGRELIYMSGRGHFPRVTTVSDLTKRNNMDFSIRISSITPADVGTYYCVKFRKGSPEDVEFKSGPGTEMALGAKPS

SEQ ID NO: 22 - protein, 119 aa, Artificial Sequence
  S79 peptide
EEELQMIQPEKLLLVTVGETATLHCTVTSLWPVGPVLWFRGVGPGRELIYRTGTGRFPRVTTVSDLTKRNNMDFSIRISSITPADVGTYYCVKFRKGSPEDVEFKSGPGTEVALGAKPS

SEQ ID NO: 23 - protein, 119 aa, Artificial Sequence
  S15 peptide
EEELQMIQPEKLLLVTVGETATLHCTVTSLLPVGPVLWFRGVGPGRELIYVSGRGHFPRVTTVSDLTKRENRDFSIRISSITPADVGTYYCVKFRKGSPEDVEFKSGPGTEMALGAKPS

SEQ ID NO: 24 - protein, 119 aa, Artificial Sequence
  S12 peptide
EEELQMIQPEKLLLVTVGETATLHCTVTSLLPVGPVLWFRGVGPGRELIYVSGRGHFPRVTTVSDLTKRENKDFSIRISSITPADVGTYYCVKFRKGSPEDVEFKSGPGTEMALGAKPS

SEQ ID NO: 25 - protein, 119 aa, Artificial Sequence
  S19 peptide
EEELQMIQPEKLLLVTVGETATLHCTVTSLLPVGPVLWFRGVGPGRELIYVSGRGHFPRVTTVSDLTKRNNRDFSIRISSITPADVGTYYCVKFRKGSPEDVEFKSGPGTEMALGAKPS

SEQ ID NO: 26 - protein, 119 aa, Artificial Sequence
  S85 peptide
EEELQMIQPEKLLLVTVGETATLHCTVTSLLPVGPVLWFRGVGPGRELIYVSGRGHFPRVTTVSDLTKRNNKDFSIRISSITPADVGTYYCVKFRKGSPEDVEFKSGPGTEMALGAKPS

SEQ ID NO: 27 - protein, 119 aa, Artificial Sequence
  S37peptide
EEELQMIQPEKLLLVTVGETATLHCTVTSLLPVGPVLWFRGVGPGRELIYMSGRGHFPRVTTVSDLTKRNNKDFSIRISSITPADVGTYYCVKFRKGSPEDVEFKSGPGTEMALGAKPS

SEQ ID NO: 28 - protein, 119 aa, Artificial Sequence
  S38 peptide
EEELQMIQPEKLLLVTVGETATLHCTVTSLLPVGPVLWFRGVGPGRELIYFSGRGHFPRVTTVSDLTKRNNMDFSIRISSITPADVGTYYCVKFRKGSPEDVEFKSGPGTEMALGAKPS

SEQ ID NO: 29 - protein, 119 aa, Artificial Sequence
  S22 peptide
EEELQMIQPEKLLLVTVGETATLHCTVTSLLPVGPVLWFRGVGPGRELIYISGRGHFPRVTTVSDLTKRNNMDFSIRISSITPADVGTYYCVKFRKGSPEDVEFKSGPGTEMALGAKPS

SEQ ID NO: 30 - protein, 119 aa, Artificial Sequence
  S29 peptide
EEELQMIQPEKLLLVTVGETATLHCTVTSLLPVGPVLWFRGVGPGRELIYLSGRGHFPRVTTVSDLTKRNNMDFSIRISSITPADVGTYYCVKFRKGSPEDVEFKSGPGTEMALGAKPS

SEQ ID NO: 31 - protein, 119 aa, Artificial Sequence
  S34 peptide
EEELQMIQPEKLLLVTVGETATLHCTVTSLLPVGPVLWFRGVGPGRELIYRSGRGHFPRVTTVSDLTKRNNMDFSIRISSITPADVGTYYCVKFRKGSPEDVEFKSGPGTEMALGAKPS

SEQ ID NO: 32 - protein, 119 aa, Artificial Sequence
  S41 peptide
EEELQMIQPEKLLLVTVGETATLHCTVTSLLPVGPVLWFRGVGPGRELIYVSGRGHFPRVTTVSDLTKRNNMDFSIRISSITPADVGTYYCVKFRKGSPEDVEFKSGPGTEMALGAKPS

SEQ ID NO: 33 - protein, 119 aa, Artificial Sequence
  S42 peptide
EEELQMIQPEKLLLVTVGETATLHCTVTSLLPVGPVLWFRGVGPGRELIYMSGRGHFPRVTTVSDLTKRNNIDFSIRISSITPADVGTYYCVKFRKGSPEDVEFKSGPGTEMALGAKPS

SEQ ID NO: 34 - protein, 119 aa, Artificial Sequence
  S43 peptide
EEELQMIQPEKLLLVTVGETATLHCTVTSLLPVGPVLWFRGVGPGRELIYMSGRGHFPRVTTVSDLTKRNNRDFSIRISSITPADVGTYYCVKFRKGSPEDVEFKSGPGTEMALGAKPS

SEQ ID NO: 35 - protein, 119 aa, Artificial Sequence
  S44 peptide
EEELQMIQPEKLLLVTVGETATLHCTVTSLLPVGPVLWFRGVGPGRELIYMSGRGHFPRVTTVSDLTKRNNVDFSIRISSITPADVGTYYCVKFRKGSPEDVEFKSGPGTEMALGAKPS

SEQ ID NO: 36 - protein, 119 aa, Artificial Sequence
  S45 peptide
EEELQMIQPEKLLLVTVGETATLHCTVTSLLPVGPVLWFRGVGPGRELIYMSGRGHFPRVTTVSDLTKRDNMDFSIRISSITPADVGTYYCVKFRKGSPEDVEFKSGPGTEMALGAKPS

SEQ ID NO: 37 - protein, 119 aa, Artificial Sequence
  S46 peptide
EEELQMIQPEKLLLVTVGETATLHCTVTSLLPVGPVLWFRGVGPGRELIYMSGRGHFPRVTTVSDLTKRENKDFSIRISSITPADVGTYYCVKFRKGSPEDVEFKSGPGTEMALGAKPS

SEQ ID NO: 38 - protein, 119 aa, Artificial Sequence
  S47 peptide
EEELQMIQPEKLLLVTVGETATLHCTVTSLLPVGPVLWFRGVGPGRELIYMSGRGHFPRVTTVSDLTKRENKDFSIRISSITPADVGTYYCVKFRKGSPEDVEFKSGPGTEVALGAKPS

SEQ ID NO: 39 - protein, 119 aa, Artificial Sequence
  S48 peptide
EEELQMIQPEKLLLVTVGETATLHCTVTSLLPVGPVLWFRGVGPGRELIYRSGRGHFPRVTTVSDLTKRNNKDFSIRISSITPADVGTYYCVKFRKGSPEDVEFKSGPGTEMALGAKPS

SEQ ID NO: 40 - protein, 119 aa, Artificial Sequence
  S49 peptide
EEELQMIQPEKLLLVTVGETATLHCTVTSLLPVGPVLWFRGVGPGRELIYRSGRGHFPRVTTVSDLTKRENKDFSIRISSITPADVGTYYCVKFRKGSPEDVEFKSGPGTEMALGAKPS

SEQ ID NO: 41 - protein, 581 aa, Artificial Sequence
  h1830-S58 containing heavy chain moiety peptide
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYMSGRGHFPRVTTVSDLTKRNNMDFSIRISSITPADVGTYYCVKFRKGSPEDVEFKSGPGTEMALGAKPS

SEQ ID NO: 42 - protein, 581 aa, Artificial Sequence
  h1830-S15 containing heavy chain moiety peptide
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYVSGRGHFPRVTTVSDLTKRENRDFSIRISSITPADVGTYYCVKFRKGSPEDVEFKSGPGTEMALGAKPS

SEQ ID NO: 43 - protein, 581 aa, Artificial Sequence
  h1830-S12 containing heavy chain moiety peptide
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYVSGRGHFPRVTTVSDLTKRENKDFSIRISSITPADVGTYYCVKFRKGSPEDVEFKSGPGTEMALGAKPS

SEQ ID NO: 44 - protein, 581 aa, Artificial Sequence
  h1830-S19 containing heavy chain moiety peptide
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYVSGRGHFPRVTTVSDLTKRNNRDFSIRISSITPADVGTYYCVKFRKGSPEDVEFKSGPGTEMALGAKPS

SEQ ID NO: 45 - protein, 581 aa, Artificial Sequence
  h1830-S85 containing heavy chain moiety peptide
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYVSGRGHFPRVTTVSDLTKRNNKDFSIRISSITPADVGTYYCVKFRKGSPEDVEFKSGPGTEMALGAKPS

SEQ ID NO: 46 - protein, 581 aa, Artificial Sequence
  h1830-S37 containing heavy chain moiety peptide
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYMSGRGHFPRVTTVSDLTKRNNKDFSIRISSITPADVGTYYCVKFRKGSPEDVEFKSGPGTEMALGAKPS

SEQ ID NO: 47 - protein, 584 aa, Artificial Sequence
  h1830-19-S79 containing heavy chain moiety peptide
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGEEELQMIQPEKLLLVTVGETATLHCTVTSLWPVGPVLWFRGVGPGRELIYRTGTGRFPRVTTVSDLTKRNNMDFSIRISSITPADVGTYYCVKFRKGSPEDVEFKSGPGTEVALGAKPS

SEQ ID NO: 48 - protein, 581 aa, Artificial Sequence
  h1830-S79 containing heavy chain moiety peptide
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLWPVGPVLWFRGVGPGRELIYRTGTGRFPRVTTVSDLTKRNNMDFSIRISSITPADVGTYYCVKFRKGSPEDVEFKSGPGTEVALGAKPS

SEQ ID NO: 49 - protein, 589 aa, Artificial Sequence
  h1830G1-19-S79 containing heavy chain moiety peptide
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGSGGGGSGGGGSGGGGEEELQMIQPEKLLLVTVGETATLHCTVTSLWPVGPVLWFRGVGPGRELIYRTGTGRFPRVTTVSDLTKRNNMDFSIRISSITPADVGTYYCVKFRKGSPEDVEFKSGPGTEVALGAKPS

SEQ ID NO: 50 - protein, 581 aa, Artificial Sequence
  h1830-S38 containing heavy chain moiety peptide
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYFSGRGHFPRVTTVSDLTKRNNMDFSIRISSITPADVGTYYCVKFRKGSPEDVEFKSGPGTEMALGAKPS

SEQ ID NO: 51 - protein, 581 aa, Artificial Sequence
  h1830-S22 containing heavy chain moiety peptide
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYISGRGHFPRVTTVSDLTKRNNMDFSIRISSITPADVGTYYCVKFRKGSPEDVEFKSGPGTEMALGAKPS

SEQ ID NO: 52 - protein, 581 aa, Artificial Sequence
  h1830-S29 containing heavy chain moiety peptide
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYLSGRGHFPRVTTVSDLTKRNNMDFSIRISSITPADVGTYYCVKFRKGSPEDVEFKSGPGTEMALGAKPS

SEQ ID NO: 53 - protein, 581 aa, Artificial Sequence
  h1830-S34 containing heavy chain moiety peptide
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYRSGRGHFPRVTTVSDLTKRNNMDFSIRISSITPADVGTYYCVKFRKGSPEDVEFKSGPGTEMALGAKPS

SEQ ID NO: 54 - protein, 581 aa, Artificial Sequence
  h1830-S41 peptide
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYVSGRGHFPRVTTVSDLTKRNNMDFSIRISSITPADVGTYYCVKFRKGSPEDVEFKSGPGTEMALGAKPS

SEQ ID NO: 55 - protein, 581 aa, Artificial Sequence
  h1830-S42 containing heavy chain moiety
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYMSGRGHFPRVTTVSDLTKRNNIDFSIRISSITPADVGTYYCVKFRKGSPEDVEFKSGPGTEMALGAKPS

SEQ ID NO: 56 - protein, 581 aa, Artificial Sequence
  h1830-S43 containing heavy chain moiety
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYMSGRGHFPRVTTVSDLTKRNNRDFSIRISSITPADVGTYYCVKFRKGSPEDVEFKSGPGTEMALGAKPS

SEQ ID NO: 57 - protein, 581 aa, Artificial Sequence
  h1830-S44 containing heavy chain moiety
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYMSGRGHFPRVTTVSDLTKRNNVDFSIRISSITPADVGTYYCVKFRKGSPEDVEFKSGPGTEMALGAKPS

SEQ ID NO: 58 - protein, 581 aa, Artificial Sequence
  h1830-S45 containing heavy chain moiety
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYMSGRGHFPRVTTVSDLTKRDNMDFSIRISSITPADVGTYYCVKFRKGSPEDVEFKSGPGTEMALGAKPS

SEQ ID NO: 59 - protein, 581 aa, Artificial Sequence
  h1830-S46 containing heavy chain moiety
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYMSGRGHFPRVTTVSDLTKRENKDFSIRISSITPADVGTYYCVKFRKGSPEDVEFKSGPGTEMALGAKPS

SEQ ID NO: 60 - protein, 581 aa, Artificial Sequence
  h1830-S47 containing heavy chain moiety
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYMSGRGHFPRVTTVSDLTKRENKDFSIRISSITPADVGTYYCVKFRKGSPEDVEFKSGPGTEVALGAKPS

SEQ ID NO: 61 - protein, 581 aa, Artificial Sequence
  h1830-S48 containing heavy chain moiety
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYRSGRGHFPRVTTVSDLTKRNNKDFSIRISSITPADVGTYYCVKFRKGSPEDVEFKSGPGTEMALGAKPS

SEQ ID NO: 62 - protein, 581 aa, Artificial Sequence
  h1830-S49 containing heavy chain moiety
QVQLQESGPGLVKPSQTLSLTCTVSGGSISDGSAYWSWIRQHPGKGLEYIGFISRAGSTYNTPSLKGRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYRSGRGHFPRVTTVSDLTKRENKDFSIRISSITPADVGTYYCVKFRKGSPEDVEFKSGPGTEMALGAKPS

SEQ ID NO: 63 - protein, 583 aa, Artificial Sequence
  h1831-19-S58 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGEEELQMIQPEKLLLVTVGETATLHCTVTSLLPVGPVLWFRGVGPGRELIYMSGRGHFPRVTTVSDLTKRNNMDFSIRISSITPADVGTYYCVKFRKGSPEDVEFKSGPGTEMALGAKPS

SEQ ID NO: 64 - protein, 580 aa, Artificial Sequence
  h1831-S15 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYVSGRGHFPRVTTVSDLTKRENRDFSIRISSITPADVGTYYCVKFRKGSPEDVEFKSGPGTEMALGAKPS

SEQ ID NO: 65 - protein, 580 aa, Artificial Sequence
  h1831-S12 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYVSGRGHFPRVTTVSDLTKRENKDFSIRISSITPADVGTYYCVKFRKGSPEDVEFKSGPGTEMALGAKPS

SEQ ID NO: 66 - protein, 580 aa, Artificial Sequence
  h1831-S19 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYVSGRGHFPRVTTVSDLTKRNNRDFSIRISSITPADVGTYYCVKFRKGSPEDVEFKSGPGTEMALGAKPS

SEQ ID NO: 67 - protein, 580 aa, Artificial Sequence
  h1831-S85 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYVSGRGHFPRVTTVSDLTKRNNKDFSIRISSITPADVGTYYCVKFRKGSPEDVEFKSGPGTEMALGAKPS

SEQ ID NO: 68 - protein, 580 aa, Artificial Sequence
  h1831-S37 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYMSGRGHFPRVTTVSDLTKRNNKDFSIRISSITPADVGTYYCVKFRKGSPEDVEFKSGPGTEMALGAKPS

SEQ ID NO: 69 - protein, 583 aa, Artificial Sequence
  h1831-19-S79 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGEEELQMIQPEKLLLVTVGETATLHCTVTSLWPVGPVLWFRGVGPGRELIYRTGTGRFPRVTTVSDLTKRNNMDFSIRISSITPADVGTYYCVKFRKGSPEDVEFKSGPGTEVALGAKPS

SEQ ID NO: 70 - protein, 580 aa, Artificial Sequence
  h1831-S38 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYFSGRGHFPRVTTVSDLTKRNNMDFSIRISSITPADVGTYYCVKFRKGSPEDVEFKSGPGTEMALGAKPS

SEQ ID NO: 71 - protein, 580 aa, Artificial Sequence
  h1831-S22 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYISGRGHFPRVTTVSDLTKRNNMDFSIRISSITPADVGTYYCVKFRKGSPEDVEFKSGPGTEMALGAKPS

SEQ ID NO: 72 - protein, 580 aa, Artificial Sequence
  h1831-S29 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYLSGRGHFPRVTTVSDLTKRNNMDFSIRISSITPADVGTYYCVKFRKGSPEDVEFKSGPGTEMALGAKPS

SEQ ID NO: 73 - protein, 580 aa, Artificial Sequence
  h1831-S34 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYRSGRGHFPRVTTVSDLTKRNNMDFSIRISSITPADVGTYYCVKFRKGSPEDVEFKSGPGTEMALGAKPS

SEQ ID NO: 74 - protein, 580 aa, Artificial Sequence
  h1831-S41 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYVSGRGHFPRVTTVSDLTKRNNMDFSIRISSITPADVGTYYCVKFRKGSPEDVEFKSGPGTEMALGAKPS

SEQ ID NO: 75 - protein, 580 aa, Artificial Sequence
  h1831-S42 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYMSGRGHFPRVTTVSDLTKRNNIDFSIRISSITPADVGTYYCVKFRKGSPEDVEFKSGPGTEMALGAKPS

SEQ ID NO: 76 - protein, 580 aa, Artificial Sequence
  h1831-S43 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYMSGRGHFPRVTTVSDLTKRNNRDFSIRISSITPADVGTYYCVKFRKGSPEDVEFKSGPGTEMALGAKPS

SEQ ID NO: 77 - protein, 580 aa, Artificial Sequence
  h1831-S44 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYMSGRGHFPRVTTVSDLTKRNNVDFSIRISSITPADVGTYYCVKFRKGSPEDVEFKSGPGTEMALGAKPS

SEQ ID NO: 78 - protein, 580 aa, Artificial Sequence
  h1831-S45 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYMSGRGHFPRVTTVSDLTKRDNMDFSIRISSITPADVGTYYCVKFRKGSPEDVEFKSGPGTEMALGAKPS

SEQ ID NO: 79 - protein, 580 aa, Artificial Sequence
  h1831-S46 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYMSGRGHFPRVTTVSDLTKRENKDFSIRISSITPADVGTYYCVKFRKGSPEDVEFKSGPGTEMALGAKPS

SEQ ID NO: 80 - protein, 580 aa, Artificial Sequence
  h1831-S47 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYMSGRGHFPRVTTVSDLTKRENKDFSIRISSITPADVGTYYCVKFRKGSPEDVEFKSGPGTEVALGAKPS

SEQ ID NO: 81 - protein, 580 aa, Artificial Sequence
  h1831-S48 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYRSGRGHFPRVTTVSDLTKRNNKDFSIRISSITPADVGTYYCVKFRKGSPEDVEFKSGPGTEMALGAKPS

SEQ ID NO: 82 - protein, 580 aa, Artificial Sequence
  h1831-S49 containing heavy chain moiety
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGGGEEELQMIQPEKLLLVTVGETATLHCTVTSLLPVGPVLWFRGVGPGRELIYRSGRGHFPRVTTVSDLTKRENKDFSIRISSITPADVGTYYCVKFRKGSPEDVEFKSGPGTEMALGAKPS

SEQ ID NO: 83 - protein, 444 aa, Artificial Sequence
  Hu5F9 heavy chain
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - protein, 219 aa, Artificial Sequence
  Hu5F9 light chain
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 351 aa, Artificial Sequence
  SIRP-CV
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 348 aa, Artificial Sequence
  S58-Fc
EEELQMIQPEKLLLVTVGETATLHCTVTSLLPVGPVLWFRGVGPGRELIYMSGRGHFPRVTTVSDLTKRNNMDFSIRISSITPADVGTYYCVKFRKGSPEDVEFKSGPGTEMALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - protein, 447 aa, Artificial Sequence
  Hu167-IgG4 AA heavy chain
QVQLVQSGAEVKKPGASVKVSCKASGYTFTSYWMHWVRQAPGQGLEWMGNIDPSDSETHYNQKFKDRVTMTRDTSISTAYMELSRLRSDDTAVYYCARWGYLGRSAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 88 - protein, 214 aa, Artificial Sequence
  Hu167-IgG4 AA light chain
DVQITQSPSSLSASVGDRVTITCRTSKSISKFLAWYQQKPGKAPKLLIYSGSTLQSGVPSRFSGSGSGTDFTLTISSLQPEDFATYYCQQHNEYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 5 aa, Artificial Sequence
  GGGGS domain
GGGGS

SEQ ID NO: 90 - protein, 16 aa, Artificial Sequence
  GS16 domain
GGGGSGGGGSGGGGGG

SEQ ID NO: 91 - protein, 17 aa, Artificial Sequence
  GS17 domain
GGGGSGGGGSGGGGSGG

SEQ ID NO: 92 - protein, 18 aa, Artificial Sequence
  GS18 domain
GGGGSGGGGSGGGGSGGG

SEQ ID NO: 93 - protein, 19 aa, Artificial Sequence
  GS19 domain
GGGGSGGGGSGGGGSGGGG

SEQ ID NO: 94 - protein, 30 aa, Artificial Sequence
  30AA-linker domain
DPALVHQRPAPPGGGGSGGGGSGGGGSGGG

SEQ ID NO: 95 - protein, 5 aa, Artificial Sequence
  GKPGS domain
GKPGS

SEQ ID NO: 96 - protein, 5 aa, Artificial Sequence
  GGGES
GGGES

SEQ ID NO: 97 - protein, 7 aa, Artificial Sequence
  9-2 H5L11 HCDR1 domain
DGSAYWS

SEQ ID NO: 98 - protein, 16 aa, Artificial Sequence
  9-2 H5L11 HCDR2 domain
FISRAGSTYNTPSLKG

SEQ ID NO: 99 - protein, 10 aa, Artificial Sequence
  9-2 H5L11 HCDR3 domain
SGGWLAPFDY

SEQ ID NO: 100 - protein, 17 aa, Artificial Sequence
  9-2 H5L11 LCDR1 domain
KSSQSLFYHSNQKHSLA

SEQ ID NO: 101 - protein, 7 aa, Artificial Sequence
  9-2 H5L11 LCDR2 domain
GASTRES

SEQ ID NO: 102 - protein, 9 aa, Artificial Sequence
  9-2 H5L11 LCDR3 domain
QQYYGYPYT

SEQ ID NO: 103 - protein, 5 aa, Artificial Sequence
  24D5 H12L61 HCDR1 domain
SYWMH

SEQ ID NO: 104 - protein, 17 aa, Artificial Sequence
  24D5 H12L61 HCDR2 domain
RITPSSGFAMYNEKFKN

SEQ ID NO: 105 - protein, 10 aa, Artificial Sequence
  24D5 H12L61 HCDR3 domain
GGSSYDYFDY

SEQ ID NO: 106 - protein, 15 aa, Artificial Sequence
  24D5 H12L61 LCDR1 domain
RASESVSIHGTHLMH

SEQ ID NO: 107 - protein, 7 aa, Artificial Sequence
  24D5 H12L61 LCDR2 domain
AASNLES

SEQ ID NO: 108 - protein, 9 aa, Artificial Sequence
  24D5 H12L61 LCDR3 domain
QQSFEDPLT

SEQ ID NO: 109 - protein, 583 aa, Artificial Sequence
  h1831-19-S37 heavy chain
QVQLVQSGAEVKKPGASVKVSCKASGYTFTSYWMHWVRQAPGQGLEWMGRITPSSGFAMYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGEEELQMIQPEKLLLVTVGETATLHCTVTSLLPVGPVLWFRGVGPGRELIYMSGRGHFPRVTTVSDLTKRNNKDFSIRISSITPADVGTYYCVKFRKGSPEDVEFKSGPGTEMALGAKPS

SEQ ID NO: 110 - protein, 348 aa, Artificial Sequence
  S37-Fc peptide
EEELQMIQPEKLLLVTVGETATLHCTVTSLLPVGPVLWFRGVGPGRELIYMSGRGHFPRVTTVSDLTKRNNKDFSIRISSITPADVGTYYCVKFRKGSPEDVEFKSGPGTEMALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 111 - protein, 218 aa, Artificial Sequence
  h1831K light chain
DIVLTQSPASLAVSPGQRATITCRASESVSIHGTHLMHWYQQKPGQPPKLLIYAASKLESGVPARFSGSGSGTDFTLTINPVEAEDTANYYCQQSFEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 7 aa, Artificial Sequence
  h1831K LCDR2 domain
AASKLES

SEQ ID NO: 113 - protein, 111 aa, Artificial Sequence
  h1831K light chain variable region
DIVLTQSPASLAVSPGQRATITCRASESVSIHGTHLMHWYQQKPGQPPKLLIYAASKLESGVPARFSGSGSGTDFTLTINPVEAEDTANYYCQQSFEDPLTFGQGTKLEIK

SEQ ID NO: 114 - protein, 348 aa, Artificial Sequence
  S79-Fc peptide
EEELQMIQPEKLLLVTVGETATLHCTVTSLWPVGPVLWFRGVGPGRELIYRTGTGRFPRVTTVSDLTKRNNMDFSIRISSITPADVGTYYCVKFRKGSPEDVEFKSGPGTEVALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 115 - protein, 348 aa, Artificial Sequence
  S15-Fc peptide
EEELQMIQPEKLLLVTVGETATLHCTVTSLLPVGPVLWFRGVGPGRELIYVSGRGHFPRVTTVSDLTKRENRDFSIRISSITPADVGTYYCVKFRKGSPEDVEFKSGPGTEMALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 116 - protein, 348 aa, Artificial Sequence
  S12-Fc peptide
EEELQMIQPEKLLLVTVGETATLHCTVTSLLPVGPVLWFRGVGPGRELIYVSGRGHFPRVTTVSDLTKRENKDFSIRISSITPADVGTYYCVKFRKGSPEDVEFKSGPGTEMALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 117 - protein, 348 aa, Artificial Sequence
  S19-Fc peptide
EEELQMIQPEKLLLVTVGETATLHCTVTSLLPVGPVLWFRGVGPGRELIYVSGRGHFPRVTTVSDLTKRNNRDFSIRISSITPADVGTYYCVKFRKGSPEDVEFKSGPGTEMALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 118 - protein, 348 aa, Artificial Sequence
  S85-Fc peptide
EEELQMIQPEKLLLVTVGETATLHCTVTSLLPVGPVLWFRGVGPGRELIYVSGRGHFPRVTTVSDLTKRNNKDFSIRISSITPADVGTYYCVKFRKGSPEDVEFKSGPGTEMALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 119 - protein, 348 aa, Artificial Sequence
  S38-Fc peptide
EEELQMIQPEKLLLVTVGETATLHCTVTSLLPVGPVLWFRGVGPGRELIYFSGRGHFPRVTTVSDLTKRNNMDFSIRISSITPADVGTYYCVKFRKGSPEDVEFKSGPGTEMALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 120 - protein, 348 aa, Artificial Sequence
  S22-Fc peptide
EEELQMIQPEKLLLVTVGETATLHCTVTSLLPVGPVLWFRGVGPGRELIYISGRGHFPRVTTVSDLTKRNNMDFSIRISSITPADVGTYYCVKFRKGSPEDVEFKSGPGTEMALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 121 - protein, 348 aa, Artificial Sequence
  S29-Fc peptide
EEELQMIQPEKLLLVTVGETATLHCTVTSLLPVGPVLWFRGVGPGRELIYLSGRGHFPRVTTVSDLTKRNNMDFSIRISSITPADVGTYYCVKFRKGSPEDVEFKSGPGTEMALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 122 - protein, 348 aa, Artificial Sequence
  S34-Fc peptide
EEELQMIQPEKLLLVTVGETATLHCTVTSLLPVGPVLWFRGVGPGRELIYRSGRGHFPRVTTVSDLTKRNNMDFSIRISSITPADVGTYYCVKFRKGSPEDVEFKSGPGTEMALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 123 - protein, 348 aa, Artificial Sequence
  S41-Fc peptide
EEELQMIQPEKLLLVTVGETATLHCTVTSLLPVGPVLWFRGVGPGRELIYVSGRGHFPRVTTVSDLTKRNNMDFSIRISSITPADVGTYYCVKFRKGSPEDVEFKSGPGTEMALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 124 - protein, 348 aa, Artificial Sequence
  S42-Fc peptide
EEELQMIQPEKLLLVTVGETATLHCTVTSLLPVGPVLWFRGVGPGRELIYMSGRGHFPRVTTVSDLTKRNNIDFSIRISSITPADVGTYYCVKFRKGSPEDVEFKSGPGTEMALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 125 - protein, 348 aa, Artificial Sequence
  S43-Fc peptide
EEELQMIQPEKLLLVTVGETATLHCTVTSLLPVGPVLWFRGVGPGRELIYMSGRGHFPRVTTVSDLTKRNNRDFSIRISSITPADVGTYYCVKFRKGSPEDVEFKSGPGTEMALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 126 - protein, 348 aa, Artificial Sequence
  S44-Fc peptide
EEELQMIQPEKLLLVTVGETATLHCTVTSLLPVGPVLWFRGVGPGRELIYMSGRGHFPRVTTVSDLTKRNNVDFSIRISSITPADVGTYYCVKFRKGSPEDVEFKSGPGTEMALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 127 - protein, 348 aa, Artificial Sequence
  S45-Fc peptide
EEELQMIQPEKLLLVTVGETATLHCTVTSLLPVGPVLWFRGVGPGRELIYMSGRGHFPRVTTVSDLTKRDNMDFSIRISSITPADVGTYYCVKFRKGSPEDVEFKSGPGTEMALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 128 - protein, 348 aa, Artificial Sequence
  S46-Fc peptide
EEELQMIQPEKLLLVTVGETATLHCTVTSLLPVGPVLWFRGVGPGRELIYMSGRGHFPRVTTVSDLTKRENKDFSIRISSITPADVGTYYCVKFRKGSPEDVEFKSGPGTEMALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 129 - protein, 348 aa, Artificial Sequence
  S47-Fc peptide
EEELQMIQPEKLLLVTVGETATLHCTVTSLLPVGPVLWFRGVGPGRELIYMSGRGHFPRVTTVSDLTKRENKDFSIRISSITPADVGTYYCVKFRKGSPEDVEFKSGPGTEVALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 130 - protein, 348 aa, Artificial Sequence
  S48-Fc peptide
EEELQMIQPEKLLLVTVGETATLHCTVTSLLPVGPVLWFRGVGPGRELIYRSGRGHFPRVTTVSDLTKRNNKDFSIRISSITPADVGTYYCVKFRKGSPEDVEFKSGPGTEMALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 131 - protein, 348 aa, Artificial Sequence
  S49-Fc peptide
EEELQMIQPEKLLLVTVGETATLHCTVTSLLPVGPVLWFRGVGPGRELIYRSGRGHFPRVTTVSDLTKRENKDFSIRISSITPADVGTYYCVKFRKGSPEDVEFKSGPGTEMALGAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 132 - protein, 124 aa, Artificial Sequence
  SIRP alpha protein extracellular domain with His tag
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSHHHHHH

SEQ ID NO: 133 - protein, 345 aa, Artificial Sequence
  TTI-621 peptide
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2023/0012428 A1
(43) Pub. Date: Jan. 12, 2023

(54) BIFUNCTIONAL FUSION PROTEIN AND PHARMACEUTICAL USE THEREOF

(71) Applicants: JIANGSU HENGRUI MEDICINE CO., LTD., Lianyungang (CN); SHANGHAI HENGRUI PHARMACEUTICAL CO., LTD., Shanghai (CN)

(72) Inventors: Xiaoling Gu, Shanghai (CN); Xin Ye, Shanghai (CN); Bing Hu, Shanghai (CN); Hu Ge, Shanghai (CN); Weikang Tao, Shanghai (CN)

(21) Appl. No.: 17/436,384

(22) PCT Filed: Mar. 5, 2020
(86) PCT No.: PCT/CN2020/077907

§ 371 Date: Sep. 3, 2021

(30) Foreign Application Priority Data
Mar. 6, 2019 (CN) 201910168433.0
May 24, 2019 (CN) 201910437477.9

(51) Int. Cl. C07K 14/705; C07K 16/28; A61P 35/00

(52) U.S. Cl. CPC C07K 14/70503; C07K 16/2827; A61P 35/00; A61K 2039/505

(57) ABSTRACT

Provided are a bifunctional fusion protein and pharmaceutical use thereof. Specifically, provided are a bifunctional fusion protein comprising an SIRPγ peptide variant and an anti-human PD-L1 antibody, an SIRPγ peptide variant, and pharmaceutical use thereof. The bifunctional fusion protein can specifically bind PD-L1 and CD47 to block the binding of PD-L1 or CD47 to a receptor or ligand thereof. In addition, also provided are preparation and application of the bifunctional fusion protein, and treatment of cancers and immune-related diseases.

### TECHNICAL FIELD

The present disclosure relates to a bifunctional fusion protein that specifically binds PD-L1 and CD47, a pharmaceutical composition comprising the bifunctional fusion protein, and use thereof as an anticancer agent.

### BACKGROUND OF THE INVENTION

The statements here only provide background information related to the present disclosure and do not necessarily constitute the prior art.

Programmed death-1 (PD-1) is a protein receptor expressed on the surface of T cells discovered in 1992, which participates in the process of cell apoptosis. PD-1 belongs to the CD28 family and has 23% amino acid homology with cytotoxic T lymphocyte antigen 4 (CTLA-4). However, the expression of PD-1 is different from that of CTLA-4, mainly on activated T cells, B cells and myeloid cells. PD-1 has two ligands, PD-L1 and PD-L2, respectively. PD-L1 is mainly expressed on T cells, B cells, macrophages and dendritic cells (DCs), and the expression on cells can be up-regulated upon activation. The expression of PD-L2 is relatively limited, mainly on antigen-presenting cells, such as activated macrophages and dendritic cells.

New research has found that high expression of PD-L1 protein is detected in human tumor tissues, such as breast cancer, lung cancer, stomach cancer, bowel cancer, kidney cancer, melanoma, and the expression level of PD-L1 is closely related to the clinic and prognosis of patients. Since PD-L1 plays the role of the second signal pathway to inhibit T cell proliferation, blocking the binding of PD-L1/PD-1 has become a very potential emerging target in the field of tumor immunotherapy.

The cell surface protein CD47 is expressed or overexpressed on many tumor types, including acute myeloid leukemia, various subtypes of B-cell non-Hodgkin's lymphoma, and many human solid tumor cells. Binding of CD47 to signal regulatory protein a (SIRPa) on macrophages is a “do not eat me” signal on the surface of tumor cells. Recent data indicate that anti-CD47 antibodies also help to improve the effective anti-tumor T cell response in immune-tolerant mice. Therefore, anti-CD47 antibodies are a new class of immune checkpoint inhibitors that regulate the innate immune system and the adaptive immune system.

Currently, there have been related CD47 patents, such as WO2016065329, WO2016109415, WO2014087248, WO2014093678, CN107849143A, CN108350048, CN106535914, WO2016023001A, CN107459578A, CN2017110167989, etc. For example, WO2016023001A describes a multispecific PD-1 mimetic peptide comprising a high-affinity PD-1 mimetic peptide and a high-affinity SIRP-α that specifically binds to CD47, and use thereof; CN107459578A describes a recombinant fusion protein comprising a SIRPα mutant and an anti-PD-L1 antibody that targets CD47 and PD-L1 molecules; CN201711016798.9 discloses a multifunctional fusion protein comprising the extracellular part of SIRPα and the extracellular part of PD-1.

However, many therapies in current preclinical and clinical studies are directed at the CD47/SIRPα interaction, including anti-CD47 antibody, SIRPα receptor protein and engineered SIRPα receptor protein, anti-SIRPα antibody and bispecific antibody, etc., and there is no related report on a multispecific fusion protein comprising a SIRPγ peptide.

SIRPγ is expressed on T cells and activated NK cells, and compared with SIRPa, SIRPγ binds CD47 with 10-fold lower affinity. The CD47-SIRPγ interaction participates in the contact between antigen presenting cells and T cells, as well as co-stimulates T cell activation and promotes T cell proliferation (Piccio et al., Blood 2005, 105, 2421-2427). In addition, the CD47-SIRPγ interaction plays a role in the transendothelial migration of T cells (Stefanisakis et al., Blood 2008, 112, 1280-1289).

### SUMMARY OF THE INVENTION

The present disclosure provides a bifunctional fusion protein comprising a SIRPγ peptide variant. Compared with the wild-type SIRPγ peptide, the SIRPγ peptide variant has significantly improved affinity to CD47.

In some embodiments, provided is a bifunctional fusion protein comprising a SIRPγ peptide variant and an anti-human PD-L1 antibody, the SIRPγ peptide variant being linked to the polypeptide chain of the anti-human PD-L1 antibody, the SIRPγ peptide variant is a SIRPγ peptide variant with a substitution mutation at position N51 relative to the wild-type SIRPγ peptide as shown in SEQ ID NO: 20. In some embodiments, the aforementioned SIRPγ peptide variant has the activity of binding to CD47 on the surface of tumor cells. Preferably, the SIRPγ peptide variant has more enhanced activity of binding to CD47 on the surface of tumor cells than the wild-type SIRPγ peptide.

In some embodiments, provided is a bifunctional fusion protein comprising a human SIRPγ peptide variant and an anti-human PD-L1 antibody, the SIRPγ peptide variant being linked to the polypeptide chain of the anti-human PD-L1 antibody,

wherein the SIRPγ peptide variant is a SIRPγ peptide variant with a substitution mutation at position N51 relative to the wild-type SIRPγ peptide as shown in SEQ ID NO: 20. In some embodiments, the aforementioned SIRPγ peptide variant has the activity of binding to CD47 on the surface of tumor cells. Preferably, the SIRPγ peptide variant has more enhanced activity of binding to CD47 on the surface of tumor cells than the wild-type SIRPγ peptide. In some embodiments, the aforementioned bifunctional fusion protein, wherein the SIRPγ peptide variant and the polypeptide chain of the anti-human PD-L1 antibody are directly linked by a peptide bond or covalently linked through a linker. Preferably, the linker can be selected from any one of the linkers shown in the group consisting of SEQ ID NO: 89-96, (GGGGS)n, (GGGES)n and (GKPGS)n, wherein n is an integer of 2 to 7.

In some embodiments, the aforementioned bifunctional fusion protein, wherein the carboxyl terminal of the SIRPγ peptide variant is linked to the amino terminal of the heavy chain variable region of the anti-human PD-L1 antibody,

or the carboxyl terminal of the SIRPγ peptide variant is linked to the amino terminal of the light chain variable region of the anti-human PD-L1 antibody,

or the carboxyl terminal of the heavy chain of the anti-human PD-L1 antibody is linked to the amino terminal of the SIRPγ peptide variant,

or the carboxyl terminal of the light chain of the anti-human PD-L1 antibody is linked to the amino terminal of the SIRPγ peptide variant.

In some preferred embodiments, the aforementioned bifunctional fusion protein, wherein the SIRPγ peptide variant is a SIRPγ peptide variant further comprising amino acid substitution(s) at one or more positions selected from the group consisiting of K19, K53, N101, L31, Q52, E54, H56, N70, M72 and M112 relative to the wild-type SIRPγ peptide.

In some preferred embodiments, the aforementioned bifunctional fusion protein, wherein the SIRPγ peptide variant is a SIRPγ peptide variant further comprising amino acid substitutions at one or more positions selected from K19, K53 and N101 relative to the wild-type SIRPγ peptide.

In some preferred embodiments, the aforementioned bifunctional fusion protein, wherein the SIRPγ peptide variant is a SIRPγ peptide variant with a N51R substitution mutation relative to the wild-type SIRPγ peptide as shown in SEQ ID NO: 20.

In some preferred embodiments, the aforementioned bifunctional fusion protein, wherein the SIRPγ peptide variant with a substitution mutation at position N51 does not substantially bind to CD47 on the surface of red blood cells, preferably, the SIRPγ peptide variant with a substitution mutation at position N51 is a SIRPγ peptide variant comprising a N51F, N51I, N51L, N51M or N51V substitution mutation.

In some preferred embodiments, the aforementioned bifunctional fusion protein, wherein the SIRPγ peptide variant is a SIRPγ peptide variant further comprising K19E, K53G and N101D substitution mutations relative to the wild-type SIRPγ peptide as shown in SEQ ID NO: 20.

In some preferred embodiments, the aforementioned bifunctional fusion protein, wherein the SIRPγ peptide variant further comprises K19E, N51V, Q52S, K53G, E54R, M72K and N101D mutations relative to the wild-type SIRPγ peptide as shown in SEQ ID NO: 20.

In some preferred embodiments, the aforementioned bifunctional fusion protein, wherein the SIRPγ peptide variant further comprises K19E, N51M, Q52S, K53G, E54R, M72K and N101D mutations relative to the wild-type SIRPγ peptide as shown in SEQ ID NO: 20.

In some preferred embodiments, the aforementioned bifunctional fusion protein, wherein the SIRPγ peptide variant is a SIRPγ peptide variant further comprising amino acid substitutions at one or more positions selected from the group consisiting of M6, V27, L30, V33, V36, L37, V42, E47, L66, T67, V92 and S98.

In some preferred embodiments, the aforementioned bifunctional fusion protein, wherein the amino acid sequence of the SIRPγ peptide variant (general formula I) is as shown in SEQ ID NO: 1:

(SEQ ID NO: 1)
EEELQMIQPE KLLLVTVGET ATLHCTVTSL X₁PVGPVLWFR
GVGPGRELIY X₂X₃GX₄GX₅FPRV TTVSDLTKRX₆ NX₇DFSIRISS
ITPADVGTYY CVKFRKGSPE DVEFKSGPGT EX₈ALGAKPS

wherein, X₁ is selected from L or W, X₂ is selected from M, V, F, I or L, X₃ is selected from Q, S or T, X₄ is selected from E, T or R, X₅ is selected from H or R, X₆ is selected from D, N or E, X₇ is selected from I, V, M, R or K, and X₈ is selected from M or V.

In some embodiments, the aforementioned bifunctional fusion protein, the amino acid sequence of the SIRPγ peptide variant (general formula II) is as shown in SEQ ID NO: 2:

(SEQ ID NO: 2)
EEELQMIQPE KLLLVTVGET ATLHCTVTSL X₁PVGPYLWFR
GVGPGRELIY RX₃GX₄GX₅FPRV TTVSDLTKRX₆ NX₇DFSIRISS
ITPADVGTYY CVKFRKGSPE DVEFKSGPGT EX₈ALGAKPS

wherein, X₁ is selected from L or W, X₃ is selected from Q, S or T, X₄ is selected from E, T or R, X₅ is selected from H or R, X₆ is selected from D, N or E, X₇ is selected from I, V, M, R or K, and X₈ is selected from M or V.

In further preferred embodiments, the aforementioned bifunctional fusion protein, wherein the SIRPγ peptide variant is as shown in the group consisiting of SEQ ID NO: 21, 22, 23, 24, 25, 26, 27, 28, 29, 30, 31, 32, 33, 34, 35, 36, 37, 38, 39 and 40, preferably as shown in SEQ ID NO: 26 or 27.

In some embodiments, the aforementioned bifunctional fusion protein, wherein the anti-human PD-L1 antibody is selected from the group consisiting of Avelumab, Atezolizumab, Durvalumab, JS-003, CS-1001, LY-3300054, KD-033, CK-301, CCX-4503, CX-072, KN-035, HRP00052, HRP00049, FAZ-053, GR-1405, KD-005, HLX-20, KL-A167, CBT-502, STI-A1014, REMD-290, BGB-A333, BCD-135 and MCLA-145.

In some embodiments, the aforementioned bifunctional fusion protein, wherein the anti-human PD-L1 antibody comprises a heavy chain variable region and a light chain variable region, wherein:

the heavy chain variable region comprises HCDR1, HCDR2 and HCDR3 regions with the same sequence(s) as those in the heavy chain variable region as shown in SEQ ID NO: 6, and

the light chain variable region comprises LCDR1, LCDR2 and LCDR3 regions with the same sequence(s) as those in the light chain variable region as shown in SEQ ID NO: 7; or

the heavy chain variable region comprises HCDR1, HCDR2 and HCDR3 regions with the same sequence(s) as those in the heavy chain variable region as shown in SEQ ID NO: 8, and

the light chain variable region comprises LCDR1, LCDR2 and LCDR3 regions with the same sequence(s) as those in the light chain variable region as shown in SEQ ID NO: 9; or

the heavy chain variable region comprises HCDR1, HCDR2 and HCDR3 regions with the same sequence(s) as those in the heavy chain variable region as shown in SEQ ID NO: 8, and

the light chain variable region comprises LCDR1, LCDR2 and LCDR3 regions with the same sequence(s) as those in the light chain variable region as shown in SEQ ID NO: 113. Furthermore, in some embodiments, the HCDR1, HCDR2 and HCDR3 regions and the LCDR1, LCDR2 and LCDR3 regions are defined by the Kabat numbering criteria.

In some embodiments, the aforementioned bifunctional fusion protein, the heavy chain variable region of the anti-human PD-L1 antibody comprises the HCDR1, HCDR2, and HCDR3 regions as shown in SEQ ID NO: 97, 98 and 99, respectively, and the light chain variable region of the anti-human PD-L1 antibody comprises the LCDR1, LCDR2 and LCDR3 regions as shown in SEQ ID NO: 100, 101 and 102, respectively, or

the heavy chain variable region of the anti-human PD-L1 antibody comprises the HCDR1, HCDR2, and HCDR3 regions as shown in SEQ ID NO: 103, 104 and 105, respectively, and the light chain variable region of the anti-human PD-L1 antibody comprises the LCDR1, LCDR2 and LCDR3 regions as shown in SEQ ID NO: 106, 107 and 108, respectively;

or the heavy chain variable region of the anti-human PD-L1 antibody comprises the HCDR1, HCDR2, and HCDR3 regions as shown in SEQ ID NO: 103, 104 and 105, respectively, and the light chain variable region of the anti-human PD-L1 antibody comprises the LCDR1, LCDR2 and LCDR3 regions as shown in SEQ ID NO: 106, 112 and 108, respectively.

In some embodiments, the aforementioned bifunctional fusion protein, the anti-human PD-L1 antibody comprises a heavy chain variable region and a light chain variable region, wherein:

the heavy chain variable region is shown in SEQ ID NO: 6, and the light chain variable region is shown in SEQ ID NO: 7; or

the heavy chain variable region is shown in SEQ ID NO: 8, and the light chain variable region is shown in SEQ ID NO: 113;

or

the heavy chain variable region is shown in SEQ ID NO: 8, and the light chain variable region is shown in SEQ ID NO: 9.

In some embodiments, the aforementioned bifunctional fusion protein, wherein the anti-human PD-L1 antibody further comprises a heavy chain constant region and a light chain constant region, preferably, the heavy chain constant region is as shown in SEQ ID NO: 10 or 11, and the light chain constant region is as shown in SEQ ID NO: 12.

In some embodiments, the aforementioned bifunctional fusion protein, wherein the anti-human PD-L1 antibody comprises a heavy chain and a light chain, wherein: the heavy chain is as shown in SEQ ID NO: 13 or 15, and the light chain is as shown in

SEQ ID NO: 14; or

the heavy chain is as shown in SEQ ID NO: 16 or 18, and the light chain is as shown in SEQ ID NO: 17; or

the heavy chain is as shown in SEQ ID NO: 16 or 18, and the light chain is as shown in SEQ ID NO: 111.

In some embodiments, the aforementioned bifunctional fusion protein, wherein the bifunctional fusion protein comprises a first polypeptide and a second polypeptide, wherein:

the first polypeptide is selected from the polypeptide as shown in any one of the group consisiting of SEQ ID NO: 41, 42, 43, 44, 45, 46, 47, 48, 49, 50, 51, 52, 53, 54, 55, 56, 57, 58, 59, 60, 61 and 62, and the second polypeptide is selected from the polypeptide as shown in SEQ ID NO: 14; or

the first polypeptide is selected from the polypeptide as shown in any one of the group consisiting of SEQ ID NO: 63, 64, 65, 66, 67, 68, 69, 70, 71, 72, 73, 74, 75, 76, 77, 78, 79, 80, 81, 82 and 109, and the second polypeptide is selected from the polypeptide as shown in SEQ ID NO: 17; or

the first polypeptide is selected from the polypeptide as shown in any one of the group consisiting of SEQ ID NO: 63, 64, 65, 66, 67, 68, 69, 70, 71, 72, 73, 74, 75, 76, 77, 78, 79, 80, 81, 82 and 109, and the second polypeptide is selected from the polypeptide as shown in SEQ ID NO: 111.

In some other embodiments of the present disclosure, provided is a SIRPγ peptide variant that is a SIRPγ peptide variant with a substitution mutation at position N51 relative to the wild-type SIRPγ peptide as shown in SEQ ID NO: 20. In some embodiments, the aforementioned SIRPγ peptide variant has the activity of binding with CD47 on the surface of tumor cells. Preferably, the SIRPγ peptide variant has more enhanced activity of binding with CD47 on the surface of tumor cells than the wild-type SIRPγ peptide.

In some preferred embodiments, the aforementioned SIRPγ peptide variant, wherein the SIRPγ peptide variant is a SIRPγ peptide variant with amino acid substitutions at one or more positions selected from the group consisiting of K19, K53, N101, L31, Q52, E54, H56, N70, M72 and M112 relative to the wild-type SIRPγ peptide.

In some preferred embodiments, the aforementioned SIRPγ peptide variant, wherein the SIRPγ peptide variant is a SIRPγ peptide variant further comprising amino acid substitutions at one or more positions selected from K19, K53 and N101 relative to the wild-type SIRPγ peptide.

In some preferred embodiments, the aforementioned SIRPγ peptide variant, wherein the SIRPγ peptide variant is a SIRPγ peptide variant with a N51R substitution mutation relative to the wild-type SIRPγ peptide as shown in SEQ ID NO: 20.

In some preferred embodiments, the aforementioned SIRPγ peptide variant, wherein the SIRPγ peptide variant with a substitution mutation at position N51 does not substantially bind to CD47 on the surface of red blood cells, preferably, the SIRPγ peptide variant with a substitution mutation at position NM is a SIRPγ peptide variant comprising N51F, N51I, N51L, N51M or N51V substitution mutation.

In some preferred embodiments, the aforementioned SIRPγ peptide variant, wherein the SIRPγ peptide variant is a SIRPγ peptide variant comprising K19E, K53G and N101D substitution mutations relative to the wild-type SIRPγ as shown in SEQ ID NO: 20.

In some preferred embodiments, the aforementioned SIRPγ peptide variant, wherein the SIRPγ peptide variant comprises K19E, N51V, Q52S, K53G, E54R, M72K and N101D mutations relative to the wild-type SIRPγ peptide as shown in SEQ ID NO: 20.

In some preferred embodiments, the aforementioned SIRPγ peptide variant, wherein the SIRPγ peptide variant comprises K19E, N51M, Q52S, K53G, E54R, M72K and N101D mutations relative to the wild-type SIRPγ peptide as shown in SEQ ID NO: 20.

In some preferred embodiments, the aforementioned SIRPγ peptide variant, wherein the SIRPγ peptide variant is a SIRPγ peptide variant further comprising amino acid substitutions at one or more positions selected from the group consisiting of M6, V27, L30, V33, V36, L37, V42, E47, L66, T67, V92 and S98.

In some preferred embodiments, the aforementioned SIRPγ peptide variant, wherein the SIRPγ peptide variant is as shown in SEQ ID NO: 1,

(SEQ ID NO: 1)
EEELQMIQPE KLLLVTVGET ATLHCTVTSL X₁PVGPVLWFR
GVGPGRELIY X₂X₃GX₄GX₅FPRV TTVSDLTKRX₆
NX₇DFSIRISS ITPADVGTYY CVKFRKGSPE DVEFKSGPGT
EX₈ALGAKPS

wherein, X₁ is selected from L or W, X₂ is selected from M, V, F, I or L, X₃ is selected from Q, S or T, X₄ is selected from E, T or R, X₅ is selected from H or R, X₆ is selected from D, N or E, X₇ is selected from I, V, M, R or K, and X₈ is selected from M or V.

In some preferred embodiments, the aforementioned SIRPγ peptide variant, the SIRPγ peptide variant is as shown in SEQ ID NO: 2:

(SEQ ID NO: 2)
EEELQMIQPE KLLLVTVGET ATLHCTVTSL X₁PVGPVLWFR
GVGPGRELIY RX₃GX₄GX₅FPRV TTVSDLTKRX₆
NX₇DFSIRISS ITPADVGTYY CVKFRKGSPE
DVEFKSGPGT EX₈ALGAKPS

wherein, X₁ is selected from L or W, X₃ is selected from Q, S or T, X₄ is selected from E, T or R, X₅ is selected from H or R, X₆ is selected from D, N or E, X₇ is selected from I, V, M, R or K, and X₈ is selected from M or V.

In some preferred embodiments, the aforementioned SIRPγ peptide variant, wherein the SIRPγ peptide variant is as shown in the group consisiting of SEQ ID NO: 21, 22, 23, 24, 25, 26, 27, 28, 29, 30, 31, 32, 33, 34, 35, 36, 37, 38, 39 and 40.

In other aspects of the present disclosure, also provided is a fusion protein comprising a SIRPγ peptide variant and an antibody Fc fragment, the SIRPγ peptide variant being the SIRPγ peptide variant according to any one described above; in some embodiments, the antibody Fc fragment is a human antibody Fc fragment; in some preferred embodiments, the antibody Fc fragment sequence is the same as the Fc fragment sequence in the heavy chain constant region as shown in SEQ ID NO: 10 or 11; in some preferred embodiments, the amino acid sequence of the fusion protein is as shown in the group consisiting of SEQ ID NO: 86, 110, 114, 115, 116, 117, 118, 119, 120, 121, 122, 123, 124, 125, 126, 127, 128, 129, 130 and 131.

In other aspects of the present disclosure, also provided is an anti-human PD-L1 antibody comprising a light chain variable region and a heavy chain variable region of antibody, the heavy chain variable region comprises HCDR1, HCDR2 and HCDR3 regions as shown in SEQ ID NO: 103, 104 and 105, respectively, and the light chain variable region comprises LCDR1, LCDR2 and LCDR3 regions as shown in SEQ ID NO: 106, 112 and 108, respectively.

In some embodiments, the aforementioned anti-human PD-L1 antibody, the heavy chain variable region is shown in SEQ ID NO: 8, and the light chain variable region is shown in SEQ ID NO: 113.

In some embodiments, the aforementioned anti-human PD-L1 antibody, wherein the anti-human PD-L1 antibody is a full-length antibody, further comprising an antibody constant region, preferably, the heavy chain constant region of the antibody is selected from the constant regions of human IgG1, IgG2, IgG3 and IgG4, the light chain constant region of the antibody is selected from the constant regions of human antibody κ and λ, chains, more preferably, the full-length antibody comprises the heavy chain constant region as shown in SEQ ID NO: 10 or 11 and the light chain constant region as shown in SEQ ID NO: 12.

In some preferred embodiments, the aforementioned anti-human PD-L1 antibody, the antibody comprises a heavy chain as shown in SEQ ID NO: 16 or 18, and a light chain as shown in SEQ ID NO: 111.

In other aspects, the present disclosure also provides a pharmaceutical composition comprising a therapeutically effective amount of the aforementioned bifunctional fusion protein, or the aforementioned SIRPγ peptide variant, or the aforementioned fusion protein, or the aforementioned anti-human PD-L1 antibody, and one or more pharmaceutically acceptable carriers, diluents, buffers or excipients. In some embodiments, the therapeutically effective amount is a unit dose of the composition comprising 0.1 to 3000 mg of the aforementioned bifunctional fusion protein, or the SIRPγ peptide variant according to the description above, or the fusion protein according to the description above, or the anti-human PD-L1 antibody according to the description above.

In other aspects, the present disclosure also provides an isolated nucleic acid molecule encoding the aforementioned bifunctional fusion protein, or encoding the aforementioned SIRPγ peptide variant.

In other aspects, the present disclosure also provides an isolated nucleic acid molecule encoding the aforementioned anti-human PD-L1 antibody.

In other aspects, the present disclosure also provides a recombinant vector comprising the aforementioned isolated nucleic acid molecule.

In other aspects, the present disclosure also provides a host cell transformed with a recombinant vector according to the description above, which is selected from prokaryotic cells and eukaryotic cells, preferably eukaryotic cells, more preferably mammalian cells or insect cells.

In other aspects, the present disclosure also provides a method for producing the aforementioned bifunctional fusion protein, or a method for producing the SIRPγ peptide variant according to the description above, or a method for producing the fusion protein according to the description above, or a method for producing the anti-human PD-L1 antibody according to the description above, the method comprises culturing the aforementioned host cell in culture medium to form and accumulate the aforementioned bifunctional fusion protein, or the SIRPγ peptide variant according to the description above, and recovering the bifunctional fusion protein or SIRPγ peptide variant, or the fusion protein according to the description above, or the anti-human PD-L1 antibody according to the description above from the culture.

In other aspects, the present disclosure also provides a method for eliminating immunosuppression-related diseases in a subject, which comprises administering to the subject a therapeutically effective amount of the aforementioned bifunctional fusion protein, or the SIRPγ peptide variant according to the description above, or the fusion protein according to the description above, or the anti-human PD-L1 antibody according to the description above, or the aforementioned pharmaceutical composition, or the aforementioned isolated nucleic acid molecule, preferably, the therapeutically effective amount is a unit dose of the composition comprising 0.1 to 3000 mg of the aforementioned bifunctional fusion protein, or the SIRPγ peptide variant according to the description above, or the anti-human PD-L1 antibody according to the description above.

In some embodiments, the PD-L1-CD47 bifunctional fusion protein, the SIRPγ peptide variant, or the fusion protein according to the description above, or the anti-human PD-L1 antibody according to the description above, is administered to individuals in single or accumulative administrations at a dose of about 10 μg/kg, about 50 μg/kg, about 100 μg/kg, about 200 μg/kg, about 300 μg/kg, about 400 μg/kg, about 500 μg/kg, about 600 μg/kg, about 700 μg/kg, about 800 μg/kg, about 900 μg/kg, about 1000 g/kg, about 1100 g/kg, 1200 g/kg, 1300 g/kg, 1400 g/kg, 1500 g/kg, 1600 g/kg, 1700 g/kg, 1800 g/kg, 1900 g/kg, about 2000 g/kg, about 3000 g/kg, about 4000 g/kg, about 5000 g/kg, about 6000 g/kg, about 7000 g/kg, about 8000 g/kg, about 9000 g/kg, about 10 mg/kg, about 20 mg/kg, about 30 mg/kg, about 40 mg/kg, about 50 mg/kg, about 60 mg/kg, about 70 mg/kg, about 80 mg/kg, about 90 mg/kg, about 100 mg/kg, about 200 mg/kg, about 300 mg/kg, about 400 mg/kg, about 500 mg/kg, about 600 mg/kg, about 700 mg/kg, about 800 mg/kg, about 900 mg/kg or about 1000 mg/kg.

In other aspects, the present disclosure also provides use of the aforementioned bifunctional fusion protein, or the SIRPγ peptide variant according to the description above, or the fusion protein according to the description above, or the anti-human PD-L1 antibody according to the description above, or the aforementioned pharmaceutical composition, or the aforementioned isolated nucleic acid molecule, in the preparation of medicament for eliminating immunosuppression-related diseases in a subject, preferably, the unit dose of pharmaceutical composition comprises 0.1 to 3000 mg of the aforementioned bifunctional fusion protein, or the aforementioned SIRPγ peptide variant, or the aforementioned anti-human PD-L1 antibody.

In other aspects, the present disclosure also provides the aforementioned bifunctional fusion protein, or the aforementioned SIRPγ peptide variant, or the fusion protein according to the description above, or the anti-human PD-L1 antibody according to the description above, or the aforementioned pharmaceutical composition, or the aforementioned isolated nucleic acid molecule used as medicament for eliminating immunosuppression-related diseases in a subject, preferably, the unit dose of the pharmaceutical composition comprises 0.1 to 3000 mg of the aforementioned bifunctional fusion protein, or the aforementioned SIRPγ peptide variant, or the aforementioned anti-human PD-L1 antibody.

In another aspect, the present disclosure also provides the aforementioned bifunctional fusion protein, or the aforementioned SIRPγ peptide variant, or the fusion protein according to the description above, or the anti-human PD-L1 antibody according to the description above, or the aforementioned pharmaceutical composition, or the aforementioned isolated nucleic acid molecule used as medicament, preferably, the unit dose of the pharmaceutical composition comprises 0.1 to 3000 mg of the aforementioned bifunctional fusion protein, or the aforementioned SIRPγ peptide variant, or the aforementioned anti-human PD-L1 antibody.

In some embodiments, eliminating the aforementioned immunosuppression-related diseases in a subject include cancer, bacterial or viral infection. The cancer includes but is not limited to carcinoma, lymphoma, blastoma, sarcoma and leukemia or lymphoid malignancy. More specific examples of the cancer include squamous cell carcinoma, myeloma, small cell lung cancer, non-small cell lung cancer (NSCLC), head and neck squamous cell carcinoma (HNSCC), glioma, Hodgkin's lymphoma, non-Hodgkin's lymphoma, diffuse large B-cell lymphoma (DLBCL), follicular lymphoma, acute lymphoblastic leukemia (ALL), acute myelocytic leukemia (AML), chronic lymphocytic leukemia (CLL), chronic myelocytic leukemia (CML), primary mediastinal large B-cell lymphoma, mantle cell lymphoma (MCL), small lymphocytic lymphoma (SLL), T-cell/histiocyte-rich large B-cell lymphoma, multiple myeloma, myeloid cell leukemia-1 protein (Mc1-1), myelodysplastic syndrome (MDS), gastrointestinal (tract) cancer, kidney cancer, ovarian cancer, liver cancer, lymphoblastic leukemia, lymphocytic leukemia, colorectal cancer, endometrial cancer, prostate cancer, thyroid cancer, melanoma, chondrosarcoma, neuroblastoma, pancreatic cancer, glioblastoma multiforme, gastric cancer, bone cancer, Ewing's sarcoma, cervical cancer, brain cancer, bladder cancer, hepatoma, breast cancer, colon cancer, hepatocellular carcinoma (HCC), clear cell renal cell carcinoma (RCC), head and neck cancer, pharyngolaryngeal cancer, hepatobiliary cancer, central nervous system cancer, esophageal cancer, malignant pleural mesothelioma, systemic light chain amyloidosis, lymphoplasmacytic lymphoma, myelodysplastic syndrome, myelodysplastic tumor, neuroendocrine tumor, Merkel cell carcinoma, testicular cancer and skin cancer.

### DESCRIPTION OF THE DRAWINGS

FIG. 1: Schematic diagram of the PD-L1-CD47 bifunctional fusion protein in some embodiments.

FIG. 2A to FIG. 2C: The binding ability test of PD-L1-CD47 bifunctional fusion protein to CD47 on the surface of human red blood cells, the negative controls (control) on the right side represent cell+secondary antibody. FIG. 2A and FIG. 2B represent the binding ability test of different PD-L1-CD47 bifunctional fusion proteins (10 μg/ml) to CD47 on the surface of red blood cells; FIG. 2C represents the binding ability test of different PD-L1-CD47 bifunctional fusion proteins (10 μg/ml and 1 μg/ml) to CD47 on the surface of red blood cells.

FIG. 3: The binding ability test of PD-L1-CD47 bifunctional fusion protein to CD47 on the surface of Raji cells, the negative control on the right side represents cell+secondary antibody.

FIG. 4: red blood cell phagocytosis mediated by PD-L1-CD47 bifunctional fusion protein.

FIG. 5A to FIG. 5B: Phagocytosis of tumor cells (Molp-8 cells) mediated by PD-L1-CD47 bifunctional fusion protein. FIG. 5A and FIG. 5B represent the phagocytosis of tumor cells mediated by different PD-L1-CD47 bifunctional fusion proteins detected in different batches of experiments.

FIG. 6: Red blood cell coagulation mediated by PD-L1-CD47 bifunctional fusion protein.

FIG. 7A to FIG. 7E: IFN-γ secretion mediated by PD-L1-CD47 bifunctional fusion protein. FIG. 7A, FIG. 7B, FIG. 7C, FIG. 7D and FIG. 7E represent the results of IFN-γ secretion mediated by different PD-L1-CD47 bifunctional fusion proteins.

FIG. 8: The effect of different PD-L1-CD47 bifunctional fusion proteins on the tumor volume of MC38/H-11-hCD47 (#5-4) tumor-bearing B-hCD274/hCD47/hSIRPα mouse model.

FIG. 9: The effect of different PD-L1-CD47 bifunctional fusion proteins on the tumor volume of MC38-hPD-L1-hCD47 tumor-bearing C57/BL-6 mouse model.

FIG. 10: The effect of different PD-L1-CD47 bifunctional fusion proteins on the tumor volume of MC38-hPD-L1 tumor-bearing C57/BL-6 mouse model.

FIG. 11: The effect of different PD-L1-CD47 bifunctional fusion proteins on the tumor volume of Molp-8 tumor-bearing nude mice in vivo model. This model focuses on investigating the anti-tumor effect of the bifunctional fusion protein on CD47-targeting pathway.

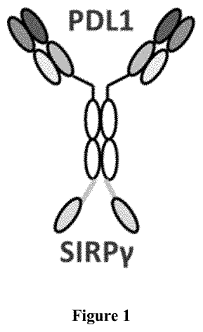

### DETAILED DESCRIPTION OF THE EMBODIMENTS

### Terms

The three-letter codes and one-letter codes of amino acids used in the present disclosure are as described in J. biol. chem, 243, p3558 (1968).

The term “bifunctional fusion protein” refers to a protein molecule that can bind to two target proteins or target antigens. The bifunctional fusion protein in the present disclosure mainly comprises a protein capable of binding to PD-L1 and CD47 on the cell surface, which is a fusion protein formed by linking an anti-PD-L1 antibody to a SIRPγ polypeptide variant.

The term “PD-L1” refers to programmed death ligand 1, also known as CD274 or B7H1. The amino acid sequence of human full-length PD-L1 is provided in GenBank under the accession number NP_054862.1. Unless specified to be from a non-human species, the term “PD-L1” means human PD-L1.

Anti-human PD-L1 antibody refers to an antibody capable of binding to human PD-L1 and capable of blocking the binding of PD-1 to PD-L1. The anti-human PD-L1 antibody can be selected from Avelumab, Atezolizumab, Durvalumab, JS-003, CS-1001, LY-3300054, KD-033, CK-301, CCX-4503, CX-072, KN-035, HRP00052, HRP00049, FAZ-053, GR-1405, KD-005, HLX-20, KL-A167, CBT-502, STI-A1014, REMD-290, BGB-A333, BCD-135, MCLA-145, etc. In addition, the anti-human PD-L1 antibody in the present disclosure can also be selected from full-length antibodies h1830, h1831, or an anti-PD-L1 antibody or antigen-binding fragment thereof that has the same CDR combination as that of the h1830 and h1831 antibodies, respectively.

“SIRPγ peptide” refers to a human SIRPγ-D1 domain peptide (the amino acid sequence of the wild-type SIRPγ peptide is shown in SEQ ID NO: 20), which has the activity of binding to human CD47. The SIRPγ peptides can also include a human SIRPγ-D1 domain peptide mutant, or a “SIRPγ peptide variant”, which is with amino acid substitutions at one or more positions relative to the wild-type SIRPγ peptide, the number of the amino acid substitution mutations is no more than 20, 19, 18, 17, 16, 15, 14, 13, 12, 11, 10, 9, 8, 7, 6, 5, 4, 3, 2, and the SIRPγ peptide variant has enhanced activity of binding to CD47 on the surface of tumor cells than the wild-type SIRPγ peptide (the affinity of the wild-type SIRPγ binding with CD47 is at micromolar level). Furthermore, in some specific embodiments, the SIRPγ peptide variant gains the property of not binding or (relative to the binding activity to CD47 on the surface of tumor cells) reduced binding to CD47 on the surface of human red blood cells. As shown in Table 1 below, for example, the S58 peptide is a mutant substituted at position K19 for K19E, position N51 for N51M, position Q52 for Q52S, position K53 for K53G, position E54 for E54R, and position N101 for N101D, relative to the wild-type SIRPγ peptide shown in SEQ ID NO: 20.

In some specific embodiments, the alternative positions of the amino acid substitution mutation can include amino acid substitution mutations at one or more position(s) selected from K19, K53, N101, L31, N51, Q52, E54, H56, N70, M72, M112, M6, V27, L30, V33, V36, L37, V42, E47, L66, T67, V92 or S98.

In some specific embodiments, the SIRPγ peptide variant is as shown in SEQ ID NO: 1:

(SEQ ID NO: 1)
EEELQMIQPE KLLLVTVGET ATLHCTVTSL X₁PVGPVLWFR
GVGPGRELIY X₂X₃GX₄GX₅FPRV TTVSDLTKRX₆ NX₇DFSIRISS
ITPADVGTYY CVKFRKGSPE DVEFKSGPGT EX₈ALGAKPS

wherein, X₁ is selected from L or W, X₂ is selected from M, V, F, I or L, X₃ is selected from Q, S or T, X₄ is selected from E, T or R, X₅ is selected from H or R, X₆ is selected from D, N or E, X₇ is selected from I, V, M, R or K, and X₈ is selected from M or V.

In some specific embodiments, the SIRPγ peptide variant is as shown in SEQ ID NO: 2:

(SEQ ID NO: 2)
EEELQMIQPE KLLLVTVGET ATLHCTVTSL X₁PVGPVLWFR
GVGPGRELIY RX₃GX₄GX₅FPRV TTVSDLTKRX₆ NX7DFSIRISS
ITPADVGTYY CVKFRKGSPE DVEFKSGPGT EX₈ALGAKPS

wherein, X₁ is selected from L or W, X₃ is selected from Q, S or T, X₄ is selected from E, T or R, X₅ is selected from H or R, X₆ is selected from D, N or E, X₇ is selected from I, V, M, R or K, and X₈ is selected from M or V.

The table below shows the amino acid substitution mutation positions and exemplary substituted amino acid residues of different SIRPγ peptide variants relative to the wild-type SIRPγ peptide.

TABLE 1

| SIRPγ peptide | | | | | | | | | | | |
|---|---|---|---|---|---|---|---|---|---|---|---|
| (wild type) | | | | | | | | | | | |
| amino acid | | | | | | | | | | | |
| residue and | | | | | | | | | | | |
| position | K19 | L31 | N51 | Q52 | K53 | E54 | H56 | N70 | M72 | N101 | M112 |
| S58 | E | | M | S | G | R | | | | D | |
| S79 | E | W | R | T | G | T | R | | | D | V |
| S15 | E | | V | S | G | R | | E | R | D | |
| S12 | E | | V | S | G | R | | E | K | D | |
| S19 | E | | V | S | G | R | | | R | D | |
| S85 | E | | V | S | G | R | | | K | D | |
| S37 | E | | M | S | G | R | | | K | D | |
| S38 | E | | F | S | G | R | | | | D | |
| S22 | E | | I | S | G | R | | | | D | |
| S29 | E | | L | S | G | R | | | | D | |
| S34 | E | | R | S | G | R | | | | D | |
| S41 | E | | V | S | G | R | | | | D | |
| S42 | E | | M | S | G | R | | | I | D | |
| S43 | E | | M | S | G | R | | | R | D | |
| S44 | E | | M | S | G | R | | | V | D | |
| S45 | E | | M | S | G | R | | D | | D | |
| S46 | E | | M | S | G | R | | E | K | D | |
| S47 | E | | M | S | G | R | | E | K | D | V |
| S48 | E | | R | S | G | R | | | K | D | |
| S49 | E | | R | S | G | R | | E | K | D | |

The term “antibody (Ab)” comprises any antigen-binding molecule or molecular complex that comprises at least one complementarity determining region (CDR) that specifically binds or interacts with a specific antigen (or epitope thereof, for example PD-L1 antigen or epitope thereof). The term “antibody” comprises: immunoglobulin molecules comprising four polypeptide chains, two heavy (H) chains and two light (L) chains, interconnected by disulfide bond(s), and multimers thereof (for example IgM). Each heavy chain comprises a heavy chain variable region (abbreviated as HCVR or VH herein) and a heavy chain constant region (CH). This heavy chain constant region comprises three regions (domains), CH1, CH2 and CH3. Each light chain comprises a light chain variable region (abbreviated as LCVR or VL herein) and a light chain constant region (CL). The light chain constant region comprises one region (domain, CL). The VH and VL regions can be further subdivided into hypervariable regions, called complementarity determining regions (CDR), between which more conservative regions (called framework regions, FR) are interspersed. Each VH and VL consists of three CDRs and four FRs, arranged from the amino terminal to the carboxyl terminal in the following order: FR1, CDR1, FR2, CDR2, FR3, CDR3, FR4. In different examples of the present disclosure, the FRs of the anti-PD-L1 antibody (or antigen-binding fragment thereof) can be the same as the human germline sequence, or can be naturally or artificially modified. The antibodies can be antibodies of different subclasses, for example, IgG (for example, IgG1, IgG2, IgG3 or IgG4 subclasses), IgA1, IgA2, IgD, IgE or IgM antibodies.

The terms “full-length antibody”, “intact antibody”, “complete antibody” and “whole antibody” are used interchangeably herein and refer to an antibody in a substantially intact form, as distinguished from the antigen-binding fragments defined below. The terms specifically refer to an antibody in which the heavy chain comprises VH region, CH1 region, hinge region and Fc region from the amino terminal to the carboxyl terminal, and the light chain comprises VL region and CL region from the amino terminal to the carboxyl terminal.

Non-limiting examples of antigen-binding fragment include: (i) Fab fragment; (ii) F(ab′)2 fragment; (iii) Fd fragment; (iv) Fv fragment; (v) single-chain Fv (scFv) molecule; (vi) dAb fragment; and (vii) the smallest recognition unit consisting of amino acid residues mimicking the antibody hypervariable region (for example isolated complementarity determining regions (CDR), for example CDR3 peptide) or restrictive FR3-CDR3-FR4 peptide. Other engineered molecules, for example region-specific antibody, single-domain antibody, region-deleted antibody, chimeric antibody, CDR-grafted antibody, diabody, triabody, tetrabody, minibody, nanobody (e.g. monovalent nanobody, bivalent nanobody, etc.), small modular immunopharmaceutical (SMIP) and shark variable IgNAR region, are also encompassed in the term “antigen-binding fragment” as used herein.

The antigen-binding fragment of an antibody will typically comprise at least one variable region. The variable region can be a region of any size or amino acid composition, and will generally comprise CDRs adjacent to one or more framework sequence(s) or within the framework. In an antigen-binding fragment with VH regions and VL regions, the VH and VL regions can be positioned opposite to each other in any suitable arrangement. For example, the variable region could be dimerized and contain VH-VL or VL-VH dimers.

In certain examples, in any configuration of the variable region and the constant region of the antigen-binding fragment, the variable region and the constant region can be directly linked to each other or can be linked through an intact or partial hinge or linker region. The hinge region can consist of at least 2 (for example 5, 10, 15, 20, 40, 60 or more) amino acids, so that it produces flexible and semi-flexible connection between adjacent variable and/or constant regions in a single polypeptide molecule. Besides, the antigen-binding fragment of the present disclosure involves homodimers or heterodimers (or other multimers) comprising variable regions and constant regions that are non-covalently linked to each other and/or linked to one or more monomer VH or VL regions (for example by disulfide bond).

“Murine antibody” in the present disclosure is a mouse or rat-derived monoclonal antibody prepared according to the knowledge and skills in the art. During preparation, the test subject is injected with antigen, and then hybridomas expressing antibodies with the desired sequence or functional properties are isolated. When the injected test subject is a mouse, the antibody produced is a mouse-derived antibody, and when the injected test subject is a rat, the antibody produced is a rat-derived antibody.

“Chimeric antibody” is an antibody formed by fusing the variable region of an antibody of the first species (such as mouse) with the constant region of an antibody of the second species (such as human). Establishing a chimeric antibody requires first establishing a hybridoma secreting monoclonal antibodies of the first species, then cloning the variable region gene from the hybridoma cells, and then cloning the antibody constant region gene of the second species as necessary, linking the variable region gene of the first species with the constant region gene of the second species to form a chimeric gene which is then inserted into an expression vector, and finally expressing the chimeric antibody molecule in a eukaryotic system or a prokaryotic system. In a preferred embodiment of the present disclosure, the antibody light chain of the chimeric antibody further comprises a light chain constant region of a human κ, λ, chain or variant thereof. The antibody heavy chain of the chimeric antibody further comprises the heavy chain constant region of human IgG1, IgG2, IgG3, IgG4 or variant thereof, preferably the heavy chain constant region of human IgG1, IgG2 or IgG4, or the heavy chain constant region variants of IgG1, IgG2 or IgG4 comprising amino acid mutations (such as YTE mutation, back mutation, L234A and/or L235A mutation or S228P mutation).

The term “humanized antibody”, including CDR-grafted antibody, refers to the antibody produced by grafting CDR sequences of an antibody derived from animals (for example murine) into the framework regions of a human antibody variable region. The humanized antibody can overcome the heterogeneous reaction induced by the chimeric antibody carrying a large amount of heterogeneous protein components. Such framework sequences can be obtained from public DNA databases or published references that include germline antibody gene sequences. For example, the germline DNA sequences of the human heavy chain and light chain variable region genes can be found in the “VBase” human germline sequence database (available on the Internet http://www.vbase2.org/), as well as in Kabat, E. A., et al., 1991, Sequences of Proteins of Immunological Interest, 5th edition. In order to avoid the decrease in activity caused by the decrease in immunogenicity, the FR sequence in human antibody variable region can be subjected to a small amount of back mutations to maintain activity. The humanized antibody of the present disclosure also includes a humanized antibody that has been further displayed by phage and subjected to affinity maturation for the CDR.

Due to the contact residues of the antigen, CDR grafting could result in reduced affinity of the produced antibody or antigen-binding fragment thereof to the antigen due to the framework residues in contact with the antigen. Such interactions could be the result of hypermutation of somatic cells. Therefore, it could still be necessary to graft such donor framework amino acids to the framework of the humanized antibody. The amino acid residues from non-human antibodies or antigen-binding fragments thereof which involve in antigen binding can be identified by examining the sequence and structure of the animal monoclonal antibody variable region. Residues in the CDR donor framework that differ from the germline can be considered related. If the closest germline cannot be determined, the sequence can be compared with the consensus sequence of a subclass or animal antibody sequence with a high percentage of similarity. Rare framework residues are thought to be the result of hypermutation of somatic cells and thus play an important role in binding.

In an embodiment of the present disclosure, the antibody or antigen-binding fragment thereof could further comprise the light chain constant region of human or murine κ, λ, chain or variant thereof, or further comprise the heavy chain constant region of human or murine IgG1, IgG2, IgG3, IgG4 or variant thereof.

“Conventional variant” of the human antibody heavy chain constant region and the human antibody light chain constant region refers to the variant of heavy chain constant region or light chain constant region derived from human that has been disclosed in the prior art and does not change the structure and function of the antibody variable region. Exemplary variants include IgG1, IgG2, IgG3 or IgG4 heavy chain constant region variants with site-directed modifications and amino acid substitutions in the heavy chain constant region. Specific substitutions are such as YTE mutations, L234A and/or L235A mutations, or S228P mutations, or mutations to obtain a knob-into-hole structure (so that the antibody heavy chain has a combination of knob-Fc and hole-Fc) known in the art. These mutations have been confirmed to make the antibody have new properties, but do not change the function of the antibody variable region.

“Human antibody” and “human-derived antibody” can be used interchangeably, and can be an antibody derived from human or an antibody obtained from a transgenic organism which is “engineered” to produce specific human antibodies in response to antigen stimulation and can be produced by any method known in the art. In some technologies, the elements of human heavy chain and light chain gene loci are introduced into cell lines in which the endogenous heavy chain and light chain gene loci are target disrupted. The transgenic organism can synthesize human antibodies specific to antigens, and the organism can be used to produce human antibody-secreting hybridomas. A human antibody can also be an antibody in which the heavy chain and light chain are encoded by nucleotide sequences derived from one or more human DNA origins. A fully human antibody can also be constructed by gene or chromosome transfection methods and phage display technology, or constructed by B cells activated in vitro, all of which are known in the art.

“Monoclonal antibody” refers to an antibody obtained from a population of substantially homogeneous antibodies, that is, except for possible variant antibodies (for example, comprising naturally-occurring mutations or mutations generated during the manufacture of monoclonal antibody preparations, these variants are usually present in a small amount), the individual antibodies constituting the population recognize the same and/or bind with the same epitope. Each monoclonal antibody of a monoclonal antibody preparation (formulation) is directed against a single determinant on an antigen. Therefore, the modifier “monoclonal” indicates the characteristics of the antibody as obtained from a substantially homogeneous antibody population, and should not be interpreted as requiring any specific method to manufacture the antibody. For example, monoclonal antibodies used according to the present disclosure can be prepared by various techniques, including but not limited to hybridoma methods, recombinant DNA methods, phage display methods, as well as methods that utilizes transgenic animals comprising the complete or partial human immunoglobulin gene loci. Such methods and other exemplary methods for preparing monoclonal antibodies are described herein.

In addition, although the two domains VL and VH of the Fv fragment are encoded by separate genes, recombination methods can be used to connect them by synthetic linkers, so that they can be produced as a single protein chain in which the VL and VH regions pair to form a monovalent molecule (referred to as single-chain Fv (scFv); see, for example, Bird et al. (1988) Science 242: 423-426; and Huston et al. (1988) Proc. Natl. Acad. Sci USA 85: 5879-5883). Such single chain antibodies are also intended to be included in the term “antigen-binding fragment” of antibody. Such antibody fragments are obtained by using conventional techniques known to those skilled in the art, and the fragments are screened for function in the same manner as that used for screening intact antibodies. The antigen binding moiety can be produced by recombinant DNA technology or by enzymatic or chemical fragmentation of the intact immunoglobulin.

The antigen-binding fragment can also be incorporated into a single-chain molecule comprising a pair of tandem Fv fragments (VH-CH1-VH-CH1), which together with a complementary light chain polypeptide forms a pair of antigen-binding regions (Zapata et al., 1995, Protein Eng. 8(10): 1057-1062; and U.S. Pat. No. 5,641,870).

Fab is an antibody fragment with a molecular weight of about 50,000 Da and with antigen-binding activity, obtained by treating an IgG antibody with the protease papain (which cleaves the amino acid residue at position 224 in the H chain), in which about half of the H chain at N-terminal side and the entire L chain are bound together by disulfide bond(s).

F(ab′)₂ is is an antibody fragment with a molecular weight of about 100,000 Da and with antigen-binding activity, obtained by digesting the downstream part of the two disulfide bonds in the hinge region of IgG with pepsin, and comprises two Fab regions linked at the hinge position.

Fab′ is an antibody fragment with a molecular weight of about 50,000 Da and with antigen-binding activity, obtained by cleaving the disulfide bond in the hinge region of the aforementioned F(ab′)2. Fab′ can be produced by treating the F(ab′)2 that specifically recognizes and binds an antigen with reducing agents, for example dithiothreitol.

In addition, Fab′ can be expressed by inserting the DNA encoding the Fab′ fragment of the antibody into a prokaryotic expression vector or a eukaryotic expression vector and introducing the vector into a prokaryotic organism or eukaryotic organism.

The term “single-chain antibody”, “single-chain Fv” or “scFv” refers to molecules comprising an antibody heavy chain variable domain (or region; VH) and an antibody light chain variable domain (or region; VL) linked by a linker. Such scFv molecules can be represented by a general formula: NH₂-VL-linker-VH-COOH or NH₂-VH-linker-VL-COOH. Suitable linkers of prior art consist of repeating GGGGS amino acid sequences or variants thereof, for example 1 to 4 (including 1, 2, 3 or 4) repeated variants (Holliger et al. (1993), Proc. Natl. Acad. Sci. USA 90: 6444-6448). Other linkers that can be used in the present disclosure are described in Alfthan et al. (1995), Protein Eng. 8:725-731, Choi et al. (2001), Eur. J. Immunol. 31:94-106, Hu et al. (1996), Cancer Res. 56:3055-3061, Kipriyanov et al. (1999), J. Mol. Biol. 293:41-56 and Roovers et al. (2001), Cancer Immunol Immunother. 50:51-59.

“Anti-human PD-L1 antibody” includes a full-length antibody capable of specifically binding to human PD-L1, as well as an antigen-binding fragment comprising the variable region of the light chain and the variable region of the heavy chain of the full-length antibody, including but not limited to a single chain antibody (scFv), Fab fragment, or other antigen-binding fragment comprising scFv or Fab comprising the light chain variable region and the heavy chain variable region of the full-length antibody.

The “linked” when used in the expression “the SIRPγ peptide is linked to the polypeptide chain of the anti-human PD-L1 antibody” refers to an effective connection between the polypeptides, including, for example, connection via a peptide bond, or connection through a linker. The connection will not lead to loss of the respective functions of the SIRPγ peptide and the anti-human PD-L1 antibody.

“Linker” refers to a connective polypeptide sequence used to connect protein domains or different proteins or different polypeptides, usually with a certain degree of flexibility. The use of linkers will not lead to loss of the original functions of the protein domains. Exemplary linkers are shown in the table below.

TABLE 2
Sequences of exemplary linkers

| | | SEQ |
|---|---|---|
| | | ID |
| Name | Sequence | NO |
| GGGGS | GGGGS | 89 |
| GS16 | GGGGSGGGGSGGGGGG | 90 |
| GS17 | GGGGSGGGGSGGGGSGG | 91 |
| GS18 | GGGGSGGGGSGGGGSGGG | 92 |
| GS19 | GGGGSGGGGSGGGGSGGGG | 93 |
| 30AA- | DPALVHQRPAPPGGGGSGGGGSGGGGSGGG | 94 |
| linker | | |
| GKPGS | GKPGS | 95 |
| GGGES | GGGES | 96 |

(GGGGS)n, n is an integer of 2 to 7
(GGGES)n, n is an integer of 2 to 7
(GKPGS)n, n is an integer of 2 to 7

In some embodiments, the anti-PD-L1 antibodies can be linked to the SIRPγ peptide variant by linker(s). Some exemplary bifunctional fusion proteins include the fusion proteins shown below:

TABLE 3
PD-L1-CD47 bifunctional fusion proteins

| | h1830 | h1830 | h1831 | h1831 | h1831 | h1831 |
|---|---|---|---|---|---|---|
| | GS16 | GS19 | GS16 | GS19 | GS16 | GS19 |
| SIRPγ | linker | linker | linker | linker | linker | linker |
| S58 | h1830-S58 | h1830-19-S58 | h1831-S58 | h1831-19-S58 | h1831K-S58 | h1831K-19-S58 |
| S15 | h1830-S15 | h1830-19-S15 | h1831-S15 | h1831-19-S15 | h1831K-S15 | h1831K-19-S15 |
| S12 | h1830-S12 | h1830-19-S12 | h1831-S12 | h1831-19-S12 | h1831K-S12 | h1831K-19-S12 |
| S19 | h1830-S19 | h1830-19-S19 | h1831-S19 | h1831-19-S19 | h1831K-S19 | h1831K-19-S19 |
| S85 | h1830-S85 | h1830-19-S85 | h1831-S85 | h1831-19-S85 | h1831K-S85 | h1831K-19-S85 |
| S37 | h1830-S37 | h1830-19-S37 | h1831-S37 | h1831-19-S37 | h1831K-S37 | h1831K-19-S37 |
| S79 | h1830-S79 | h1830-19-S79 | h1831-S79 | h1831-19-S79 | h1831K-S79 | h1831K-19-S79 |
| S38 | h1830-S38 | h1830-19-S38 | h1831-S38 | h1831-19-S38 | h1831K-S38 | h1831K-19-S38 |
| S22 | h1830-S22 | h1830-19-S22 | h1831-S22 | h1831-19-S22 | h1831K-S22 | h1831K-19-S22 |
| S29 | h1830-S29 | h1830-19-S29 | h1831-S29 | h1831-19-S29 | h1831K-S29 | h1831K-19-S29 |
| S34 | h1830-S34 | h1830-19-S34 | h1831-S34 | h1831-19-S34 | h1831K-S34 | h1831K-19-S34 |
| S41 | h1830-S41 | h1830-19-S41 | h1831-S41 | h1831-19-S41 | h1831K-S41 | h1831K-19-S41 |
| S42 | h1830-S42 | h1830-19-S42 | h1831-S42 | h1831-19-S42 | h1831K-S42 | h1831K-19-S42 |
| S43 | h1830-S43 | h1830-19-S43 | h1831-S43 | h1831-19-S43 | h1831K-S43 | h1831K-19-S43 |
| S44 | h1830-S44 | h1830-19-S44 | h1831-S44 | h1831-19-S44 | h1831K-S44 | h1831K-19-S44 |
| S45 | h1830-S45 | h1830-19-S45 | h1831-S45 | h1831-19-S45 | h1831K-S45 | h1831K-19-S45 |
| S46 | h1830-S46 | h1830-19-S46 | h1831-S46 | h1831-19-S46 | h1831K-S46 | h1831K-19-S46 |
| S47 | h1830-S47 | h1830-19-S47 | h1831-S47 | h1831-19-S47 | h1831K-S47 | h1831K-19-S47 |
| S48 | h1830-S48 | h1830-19-S48 | h1831-S48 | h1831-19-S48 | h1831K-S48 | h1831K-19-S48 |
| S49 | h1830-S49 | h1830-19-S49 | h1831-S49 | h1831-19-S49 | h1831K-S49 | h1831K-19-S49 |

Diabody refers to an antibody fragment of dimerized scFv, and is an antibody fragment with bivalent antigen binding activity. In the bivalent antigen binding activity, the two antigens can be the same or different.

The dsFv is obtained by connecting polypeptides (in which one amino acid residue in each of VH and VL is substituted with a cysteine residue) via a disulfide bond between cysteine residues. The amino acid residues substituted with cysteine residues can be selected according to a known method (Protein Engineering. 7:697 (1994)) based on the three-dimensional structure prediction of the antibody.

The antigen-binding fragment in some examples of the present disclosure can be produced by the following steps: obtaining the cDNA encoding the VH and/or VL and other required domains of the monoclonal antibody of the present disclosure that specifically recognizes and binds to an antigen, constructing the DNA encoding the antigen-binding fragment, inserting the DNA into a prokaryotic expression vector or a eukaryotic expression vector, and then introducing the expression vector into a prokaryote or eukaryote to express the antigen-binding fragment.

“Fc region” can be a native sequence Fc region or a variant Fc region. Although the boundaries of the Fc region of an immunoglobulin heavy chain could vary, the Fc region of human IgG heavy chain is usually defined as extending from the amino acid residue at position Cys226 or from Pro230 to its carboxyl terminal. The numbering of residues in the Fc region is as the numbering of the EU index in Kabat. Kabat et al., Sequences of Proteins of Immunological Interest, 5th Edition, Public Health Service, National Institutes of Health, Bethesda, Md., 1991. The Fc region of immunoglobulin usually has two constant region domains, CH2 and CH3.

The term “amino acid difference” or “amino acid mutation” refers to the presence of amino acid changes or mutations in the variant protein or polypeptide compared with the original protein or polypeptide, including insertion, deletion or substitution of one or more amino acid, on the basis of the original protein or polypeptide.

“Variable region” of an antibody refers to the antibody light chain variable region (VL) or the antibody heavy chain variable region (VH), alone or in combination. As known in the art, the variable regions of the heavy chain and the light chain each consist of 4 framework regions (FRs) linked by 3 complementarity determining regions (CDRs) (also called hypervariable regions). The CDRs in each chain are held tightly together by FRs, and contribute to the formation of the antigen binding site of the antibody together with the CDRs from the other chain. At least two techniques for determining CDR can be mentioned: (1) a method based on cross-species sequence variability (i.e., Kabat et al., Sequences of Proteins of Immunological Interest, (5th edition, 1991, National Institutes of Health, Bethesda Md.)); and (2) a method based on the crystallographic study of antigen-antibody complexes (Al-Lazikani et al., J. Molec. Biol. 273:927-948 (1997)). As used herein, CDR can refer to a CDR determined by either method or a combination of the two methods.

The term “antibody framework” or “FR region” refers to a moiety of the variable domain VL or VH, which serves as a scaffold for the antigen binding loop (CDR) of the variable domain. Essentially, it is a variable domain without CDR.

The term “complementarity determining region” and “CDR” refer to one of the six hypervariable regions in the variable domain of an antibody that mainly contribute to antigen binding. Generally, three CDRs (HCDR1, HCDR2, HCDR3) are present in each heavy chain variable region, and three CDRs (LCDR1, LCDR2, LCDR3) are present in each light chain variable region. Any one of a variety of well-known schemes can be used to determine the amino acid sequence boundaries of a CDR, including the “Kabat” numbering criteria (see Kabat et al. (1991), “Sequences of Proteins of Immunological Interest”, 5th edition, Public Health Service, National Institutes of Health, Bethesda, Md.), “Chothia” numbering criteria (Al-Lazikani et al., (1997) JMB 273:927-948) and ImMunoGenTics (IMGT) numbering criteria (Lefranc M. P., Immunologist, 7, 132-136 (1999); Lefranc, M. P. et al., Dev. Comp. Immunol., 27, 55-77 (2003)), etc. For example, for the classical format, following the Kabat criteria, the CDR amino acid residue numbers in the heavy chain variable domain (VH) are 31-35 (HCDR1), 50-65 (HCDR2) and 95-102 (HCDR3); the CDR amino acid residue numbers in the light chain variable domain (VL) are 24-34 (LCDR1), 50-56 (LCDR2) and 89-97 (LCDR3). Following the Chothia criteria, the CDR amino acid numbers in VH are 26-32 (HCDR1), 52-56 (HCDR2) and 95-102 (HCDR3); and the amino acid residue numbers in VL are 26-32 (LCDR1), 50-52 (LCDR2) and 91-96 (LCDR3). By combining the CDR definitions of both Kabat and Chothia, the CDR consists of amino acid residues 26-35 (HCDR1), 50-65 (HCDR2) and 95-102 (HCDR3) in human VH and amino acid residues 24-34 (LCDR1), 50-56 (LCDR2) and 89-97 (LCDR3) in human VL. Following IMGT criteria, the CDR amino acid residue numbers in VH are roughly 26-35 (CDR1), 51-57 (CDR2) and 93-102 (CDR3), and the CDR amino acid residue numbers in VL are roughly 27-32 (CDR1), 50-52 (CDR2) and 89-97 (CDR3). Following IMGT criteria, the CDR region of an antibody can be determined by using the program IMGT/DomainGap Align.

“Antibody constant region domain” refers to the domain derived from the constant regions of the light chain and heavy chain of an antibody, including CL and CH1, CH2, CH3 and CH4 domains derived from different types of antibodies.

“Epitope” or “antigenic determinant” refers to a site on an antigen to which an immunoglobulin or an antibody specifically binds. Epitopes usually include at least 3, 4, 5, 6, 7, 8, 9, 10, 11, 12, 13, 14 or 15 consecutive or non-consecutive amino acids in a unique spatial conformation. See, for example, Epitope Mapping Protocols in Methods in Molecular Biology, Vol. 66, G. E. Morris, Ed. (1996).

The terms “specifically bind”, “selectively bind”, “selective binding” and “specific binding” refer to the binding of an antibody to an epitope on a predetermined antigen.

The term “affinity” refers to the strength of the interaction between an antibody and an antigen at a single epitope. Within each antigenic site, the variable region of the antibody “arm” interacts with the antigen at multiple amino acid positions through weak non-covalent forces; the greater the interaction, the stronger the affinity. As used herein, the term “high affinity” of an antibody or antigen-binding fragment thereof (e.g. Fab fragment) generally refers to an antibody or antigen-binding fragment with a KD of 1E⁻⁹M or less (e.g., a KD of 1E⁻¹⁰ M or less, a KD of 1E⁻¹¹M or less, a KD of 1E⁻¹²M or less, a K_{D} of 1E⁻¹³M or less, a KD of 1E⁻¹⁴M or less, etc.).

The term “KD” or “KD” refers to the dissociation equilibrium constant of a specific antibody-antigen interaction. Generally, an antibody binds to an antigen with a dissociation equilibrium constant (KD) of less than about 1E⁻⁸M, for example, less than about 1E⁻⁹M, 1E⁻¹⁹M or 1E⁻¹¹M or less, for example, as measured in a BIACORE instrument using surface plasmon resonance (SPR) technology. The smaller the KD value, the greater the affinity is.

The term “nucleic acid molecule” refers to DNA molecules and RNA molecules. A nucleic acid molecule can be a single-stranded or double-stranded DNA molecule or RNA molecule, for example, a double-stranded DNA or mRNA. When a nucleic acid is placed in a functional relationship with another nucleic acid sequence, the nucleic acid is “operatively linked”. For example, if a promoter or enhancer affects the transcription of a coding sequence, then the promoter or enhancer is operatively linked to the coding sequence.

The term “vector” means a construct capable of delivering one or more target genes or sequences and preferably expressing the same in a host cell. Examples of vectors include, but are not limited to, viral vector, naked DNA or RNA expression vector, plasmid, cosmid or phage vector, DNA or RNA expression vector associated with cationic flocculant, DNA or RNA expression vector encapsulated in liposome, and certain eukaryotic cell such as producer cell.

The methods for producing and purifying antibodies and antigen-binding fragments are well known in the prior art, such as Antibodies: A Laboratory Manual, Cold Spring Harbor, Chapters 5-8 and 15. For example, mice can be immunized with an antigen or fragment thereof, and the obtained antibody can be renatured and purified, and amino acid sequencing can be performed by using conventional methods. Antigen-binding fragments can also be prepared by using conventional methods. The antibody or antigen-binding fragment according to the present disclosure is genetically engineered to add one or more human FR regions onto the non-human CDR regions. The human FR germline sequences can be obtained from the website http://www.imgt.org/ by aligning against the IMGT human antibody variable region germline gene database via MOE software, or be obtained from The Immunoglobulin FactsBook, 2001ISBN012441351.

The term “host cell” refers to a cell into which an expression vector has been introduced. Host cells can include bacteria, microorganisms, plant or animal cells. Bacteria that can be easily transformed include members of the enterobacteriaceae, for example Escherichia coli or Salmonella strains; Bacillaceae, for example Bacillus subtilis; Pneumococcus; Streptococcus and Haemophilus influenzae. Suitable microorganisms include Saccharomyces cerevisiae and Pichia pastoris. Suitable animal host cell lines include CHO (Chinese Hamster Ovary Cell Line), HEK293 cells (non-limiting examples such as HEK293E cells) and NSO cells.

The engineered antibody or antigen-binding fragment can be prepared and purified by conventional methods. For example, the cDNA sequences encoding the heavy chain and light chain can be cloned and recombined into a GS expression vector. The recombinant immunoglobulin expression vectors can be stably transfected into CHO cells. As an alternative prior art, mammalian expression systems can lead to glycosylation of antibodies, especially at highly conserved N-terminal positions of the Fc region. Stable clones are obtained by expressing antibodies that specifically bind with antigens. Positive clones are expanded in serum-free medium of bioreactors to produce antibodies. The culture medium into which the antibodies are secreted can be purified by conventional techniques. For example, Protein A or Protein G Sepharose FF column comprising adjusted buffer can be used for purification. Non-specifically bound components are washed away. Then the bound antibodies are eluted by the pH gradient, and the antibody fragments are detected by SDS-PAGE and collected. The antibodies can be filtered and concentrated by conventional methods. Soluble mixtures and multimers can also be removed by conventional methods, for example molecular sieves and ion exchange. The resulting product needs to be frozen immediately, such as at −70° C., or lyophilized.

“Administering”, “administration”, “giving” and “treating”, when applied to animals, humans, experimental subjects, cells, tissues, organs or biological fluids, refer to the contact of exogenous medicament, therapeutic agent, diagnostic agent, composition or manual operation (for example “euthanasia” in the example) with the animals, humans, subjects, cells, tissues, organs or biological fluids. “Giving” and “treating” can refer to for example treatment, pharmacokinetics, diagnosis, research and experimental methods. The treatment of cells includes contact of reagents with cells, and contact of reagents with fluids, in which the fluids are in contact with the cells. “Giving” and “treating” also mean treating for example cells by reagents, diagnostic agent, binding compositions or by another kind of cells in vitro and ex vivo. “Treating” when applied to human, veterinary or research subjects, refers to therapeutic treatment, prevention or prophylactic measures, research and diagnostic applications.

“Treatment” means applying an internal or external therapeutic agent, for example a composition comprising any one of the compounds of the present disclosure, to a patient (or subject) who has (or is suspected of having, or is susceptible to) one or more disease symptoms on which the therapeutic agent is known to have therapeutic effect. Generally, the therapeutic agent is given at an amount effective to alleviate one or more disease symptoms in the treated patient (or subject) or population to induce the regression of such symptoms or inhibit the development of such symptoms to any clinically detectable extent. The amount of therapeutic agent that is effective to alleviate any specific disease symptom (also referred to as a “therapeutically effective amount”) can vary according to a variety of factors, for example the patient's (or subject's) disease state, age and body weight, and the ability of the agent to produce the desired therapeutic effect in the patient (or subject). Whether the disease symptoms have been alleviated can be evaluated through any clinical testing methods commonly used by doctors or other health care professionals to evaluate the severity or progression of the symptoms. Although the embodiments of the present disclosure (for example treatment methods or products) are ineffective in alleviating each target disease symptom, but shall reduce the target disease symptom in a statistically significant number of patients (or subjects), as determined according to any statistical test methods known in the art, such as Student t-test, chi-square test, Mann and Whitney's U test, Kruskal-Wallis test (H test), Jonckheere-Terpstra test and Wilcoxon test.

“Amino acid conservative modification” or “amino acid conservative substitution” refers to the substitution of amino acids in a protein or polypeptide with other amino acids with similar characteristics (for example charge, side chain size, hydrophobicity/hydrophilicity, main chain conformation and rigidity, etc.), thereby allowing frequent changes without changing the biological activity or other required characteristics (for example antigen affinity and/or specificity) of the protein or polypeptide. Those skilled in the art know that, generally, a single amino acid substitution in a non-essential region of a polypeptide does not substantially change the biological activity (see, for example, Watson et al., (1987) Molecular Biology of the Gene, The Benjamin/Cummings Pub. Co., Page 224, (4th edition)). In addition, the substitution of amino acids with similar structure or function is unlikely to disrupt the biological activity. Exemplary conservative substitutions are stated in the table “Exemplary conservative substitutions of amino acid” below.

TABLE 4
Exemplary conservative substitutions of amino acid

| Original residue | Conservative substitution |
|---|---|
| Ala(A) | Gly; Ser |
| Arg(R) | Lys; His |
| Asn(N) | Gln; His; Asp |
| Asp(D) | Glu; Asn |
| Cys(C) | Ser; Ala; Val |
| Gln(Q) | Asn; Glu |
| Glu(E) | Asp; Gln |
| Gly(G) | Ala |
| His(H) | Asn; Gin |
| Ile(I) | Leu; Val |
| Leu(L) | Ile; Val |
| Lys(K) | Arg; His |
| Met(M) | Leu; Ile; Tyr |
| Phe(F) | Tyr; Met; Leu |
| Pro(P) | Ala |
| Ser(S) | Thr |
| Thr(T) | Ser |
| Trp(W) | Tyr; Phe |
| Tyr(Y) | Trp; Phe |
| Val(V) | Ile; Leu |

“Effective amount”, “effective dose” refers to the amount of an agent, compound or pharmaceutical composition necessary to obtain any one or more beneficial or desired therapeutic results. For prophylactic use, the beneficial or desired results include elimination or reduction of risk, reduction of severity or delay of the disease onset, including the biochemistry, histology and/or behavioral symptoms of the disease, complications thereof and intermediate pathological phenotypes that appear during the developmental process of the disease. For therapeutic applications, the beneficial or desired results include clinical results, for example reducing the incidence of various target antigen-related disorders of the present disclosure or improving one or more symptoms of the disorder, reducing the dose of other agents required to treat the disorder, enhancing the therapeutic effect of another agent, and/or delaying the progression of target antigen-related disorder of the present disclosure in the patient (or subject).

“Exogenous” refers to substances produced outside organisms, cells or human bodies according to circumstances.

“Endogenous” refers to substances produced inside cells, organisms or human bodies according to circumstances.

“Isolated” refers to a purified state, and in this case means that the designated molecule is substantially free of other biomolecules, for example nucleic acids, proteins, lipids, carbohydrates or other materials, for example cell debris and growth medium. Generally, the term “isolated” is not intended to mean the complete absence of these materials or the absence of water, buffer or salt, unless they are present in an amount that significantly interferes with the experimental or therapeutic use of the compound as described herein.

“Optional” or “optionally” means that the event or environment that follows the term can but does not have to occur, and this description includes occasions where the event or environment occurs or does not occur.

“Pharmaceutical composition” means a mixture comprising one or more of the compounds described in the present disclosure, or a physiologically/pharmaceutically acceptable salt or a prodrug thereof, and other chemical compositions, for example physiological/pharmaceutically acceptable carriers and excipients. The purpose of the pharmaceutical composition is to promote the administration to organisms, which facilitates the absorption of the active ingredient and thereby exerts biological activity.

The term “pharmaceutically acceptable carrier” refers to any inactive substance suitable for use in a formulation for the delivery of antibodies or antigen-binding fragments. The carrier can be an anti-adhesive agent, binder, coating, disintegrant, filler or diluent, preservative (such as antioxidant, antibacterial or antifungal agent), sweetener, absorption delaying agent, wetting agent, emulsifier, buffer, etc. Examples of suitable pharmaceutically acceptable carriers include water, ethanol, polyol (for example glycerol, propanediol, polyethylene glycol, etc.) dextrose, vegetable oil (for example olive oil), saline, buffer, buffered saline, and isotonic agent for example saccharide, polyol, sorbitol and sodium chloride.

In addition, another aspect of the present disclosure relates to methods for immunodetection or determination of target antigens, reagents for immunodetection or determination of target antigens, methods for immunodetection or determination of cells expressing target antigens and diagnostic agents for diagnosing diseases related to target antigen-positive cells, which includes the monoclonal antibody or antibody fragment, or fusion protein, or bifunctional fusion protein of the present disclosure (which specifically recognizes and binds target antigen) used as an active ingredient.

The terms “cancer”, “cancerous” or “malignant tumor” refer to or describe the physiological condition in mammals that is generally characterized by unregulated cell growth, and are used interchangeably in the present disclosure. Examples of the cancer or malignant tumor include but are not limited to carcinoma, lymphoma, blastoma, sarcoma and leukemia or lymphoid malignancy. More specific examples of the cancer include squamous cell carcinoma, myeloma, small cell lung cancer, non-small cell lung cancer (NSCLC), head and neck squamous cell carcinoma (HNSCC), glioma, Hodgkin's lymphoma, non-Hodgkin's lymphoma, diffuse large B-cell lymphoma (DLBCL), follicular lymphoma, acute lymphoblastic leukemia (ALL), acute myelocytic leukemia (AML), chronic lymphocytic leukemia (CLL), chronic myelocytic leukemia (CML), primary mediastinal large B-cell lymphoma, mantle cell lymphoma (MCL), small lymphocytic lymphoma (SLL), T-cell/histiocyte-rich large B-cell lymphoma, multiple myeloma, myeloid cell leukemia-1 protein (Mcl-1), myelodysplastic syndrome (MDS), gastrointestinal (tract) cancer, kidney cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, gastric cancer, bone cancer, Ewing's sarcoma, cervical cancer, brain cancer, gastric cancer, bladder cancer, hepatoma, breast cancer, colon cancer, hepatocellular carcinoma (HCC), clear cell renal cell carcinoma (RCC), head and neck cancer, pharyngolaryngeal cancer, hepatobiliary cancer, central nervous system cancer, esophageal cancer, malignant pleural mesothelioma, systemic light chain amyloidosis, lymphoplasmacytic lymphoma, myelodysplastic syndrome, myelodysplastic tumor, neuroendocrine tumor, Merkel cell carcinoma, testicular cancer and skin cancer.

“Inflammatory disorder” refers to any disease, disorder or syndrome in which an excessive or unregulated inflammatory response results in excessive inflammatory symptoms, host tissue damage or loss of tissue function. “Inflammatory disease” also refers to a pathological state mediated by the chemotactic pooling of leukocytes or neutrophils.

“Inflammation” refers to a local protective response caused by tissue damage or destruction, which is used to destroy, weaken or eliminate (isolate) harmful substances and injured tissues. Inflammation is significantly related to the chemotactic pooling of leukocytes or neutrophils. Inflammation can be caused by pathogenic organisms and viruses as well as non-infectious causes such as trauma or reperfusion or stroke after myocardial infarction, immune response to exogenous antigens, and autoimmune response.

The aforementioned diseases related to target antigen-positive cells can be diagnosed by detecting or measuring cells expressing the target antigen using the monoclonal antibody or antibody fragment of the present disclosure.

In order to detect cells expressing the polypeptide, known immunodetection methods can be used, preferably using immunoprecipitation, fluorescent cell staining, immunohistochemical staining, etc. In addition, fluorescent antibody staining method utilizing the FMAT8100HTS system (Applied Biosystem) can be used.

In the present disclosure, there is no particular limitation on the in vivo sample used for detection or measurement of the target antigen, as long as it has the possibility of comprising cells expressing the target antigen, for example histocyte, blood, plasma, serum, pancreatic juice, urine, feces, tissue fluid or culture fluid.

According to the required diagnostic method, the diagnostic agent comprising the monoclonal antibody or antibody fragment thereof of the present disclosure can further comprise reagents for performing antigen-antibody reaction or reagents for detecting the reaction. The reagents used to perform the antigen-antibody reaction include buffer, salt, etc. The reagents used for detection include reagents commonly used in immunodetection or measurement methods, for example labeled second antibodies that recognize the monoclonal antibody, antibody fragment thereof or conjugate thereof, and a substrate corresponding to the label, etc.

The details of one or more embodiments of the present invention are presented in the above specification. Although any methods and materials similar or identical to those described herein can be used in the practice or testing of the present disclosure, the preferred methods and materials are described below. The other features, purposes and advantages of the present disclosure will be apparent through the specification and the claims. In the specification and the claims, unless otherwise clearly indicated in the context, the singular form includes the cases of plural form. Unless otherwise defined, all technical and scientific terms used herein have the general meanings understood by those of ordinary skill in the art to which the present invention belongs. All patents and publications cited in the specification are incorporated by reference. The following examples are provided to more fully illustrate the preferred embodiments disclosed in the present invention. These examples should not be construed as limiting the scope of the present disclosure in any way, and the scope of the present disclosure is defined by the claims.

### EXAMPLES

### Example 1. Preparation of CD47 Antigen and Proteins for Detection

UniProt leukocyte surface antigen CD47 (human CD47 protein, Uniprot number: Q08722) was used as a template for CD47. The amino acid sequence of the antigen and proteins for detection involved in the present disclosure were designed, alternatively different tags (such as his tag or Fc, etc.) were fused on the basis of the CD47 protein.

1. The extracellular domain of CD47 protein with His tag (CD47-ECD-His): (SEQ ID NO: 3)

QLLFNKTKSVEFTFCNDTVVIPCFVTNMEAQNTTEVYVKWKFKGRDIYTF
DGALNKSTVPTDFSSAKIEVSQLLKGDASLKMDKSDAVSHTGNYTCEVTE
LTREGETIIELKYRVVSWFSPNEHHHHHH

Note: the underlined part was a 6×his tag.

2. CD47 extracellular domain and human IgG1Fc fusion protein (CD47-ECD-Fc) as a detection reagent: (SEQ ID NO: 4)

QLLFNKTKSVEFTFCNDTVVIPCFVTNMEAQNTTEVYVKWKFKGRDIYTF
DGALNKSTVPTDFSSAKIEVSQLLKGDASLKMDKSDAVSHTGNYTCEVTE
LTREGETIIELKYRVVSWFSPNEEPKSSDKTHTCPPCPAPELLGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLS
LSPGK

Note: the underlined part was the human IgG1-Fc moiety.

3. Fusion protein of human SIRPα and human IgG1Fc (SIRPα-Fc) as a binding and blocking detection reagent: (SEQ ID NO: 5)

EEELQIIQPDKSVSVAAGESAILHCTITSLFPVGPIQWFRGAGPARVLIY
NQRQGPFPRVTTVSETTKRENMDFSISISNITPADAGTYYCIKFRKGSPD
TEFKSGAGTELSVRAKPSEPKSSDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK

Note: the underlined part was the human IgG1-Fc moiety.

4. The extracellular domain of SIRPα protein with His tag (SIRPα-His)

(SEQ ID NO: 132)
EEELQVIQPDKSVSVAAGESAILHCTVTSLIPVGPIQWFRGAGPARELIY
NQKEGHFPRVTTVSESTKRENMDFSISISNITPADAGTYYCVKFRKGSPD
TEFKSGAGTELSVRAKPSHHHHHH.

### Example 2. Purification of CD47 and SIRPα-Related Recombinant Proteins

1. Purification Steps of Recombinant Proteins with His Tag:

The cell expression supernatant was centrifuged at high speed to remove impurities, the buffer was replaced with PBS, and imidazole was added to a final concentration of 5 mM. The nickel column was equilibrated with PBS solution comprising 5 mM imidazole, and the column volume was washed 2 to 5 times. The supernatant sample after replacement was loaded onto the IMAC column. The column was washed with PBS solution comprising 5 mM imidazole until the A280 reading dropped to baseline. Then the chromatography column was washed with PBS+10 mM imidazole to remove non-specifically bound protein impurities, and the effluent was collected. The target protein was then eluted with PBS solution comprising 300 mM imidazole, and the elution peak was collected. The collected eluate was concentrated and further purified by gel chromatography Superdex200 (GE, 28-9893-35), and the mobile phase was PBS. The multimer peak was removed and the elution peak was collected. The obtained protein was identified by electrophoresis, peptide map and LC-MS, and then aliquoted for use.

The obtained CD47-ECD-His (SEQ ID NO: 3) with His tag was used as an immunogen or detection reagent for the antibody of the present disclosure. CD47-ECD-His could also be used as an immunogen to stimulate mouse immunity after coupling reaction with KLH protein by in vitro chemical methods.

2. Purification Steps of CD47-ECD-Fc and SIRPα-Fc Fusion Protein:

The cell expression supernatant was centrifuged at high speed to remove impurities, and the supernatant was subjected to MabSelect Sure (GE, 17-5438-01) affinity chromatography. The MabS elect Sure column was first regenerated with 0.1 M NaOH, washed with pure water and then equilibrated with PBS. After the binding of the supernatant, the column was washed with PBS until the A280 reading dropped to the baseline. The target protein was eluted with 0.1 M acetate buffer at pH 3.5, and neutralized with 1 M Tris-HCl. The eluted sample was properly concentrated and then further purified by PBS-balanced gel chromatography Superdex200 (GE, 28-9893-35). The collection tubes with the target protein were pooled and concentrated to an appropriate concentration.

This method was used to purify the CD47-ECD-Fc (SEQ ID NO: 4) and SIRPα-Fc (SEQ ID NO: 5) fusion protein. This method could also be used to purify the humanized antibody proteins involved in the present disclosure.

### Example 3. Construction and Expression of Anti-PD-L1 Humanized Antibody (IgG4-S228P Form)

The light and heavy chain variable regions of the anti-PD-L1 antibody were modified from the anti-PD-L1 antibody of WO2017084495A1, its sequence and related properties recorded in the PCT application with application number PCT/CN2019/070982, the entire content of which is incorporated into the present application.

Anti PD-L1 antibody 9-2 H5L11:

9-2 H5 heavy chain variable region
(SEQ ID NO: 6)
QVQLQESGPGLVKPSQTLSLTCTVSGGSISDGSAYWSWIRQHPGKGLEYI
GFISRAGSTYNTPSLKGRVTISRDTSKNQFSLKLSSVTAADTAVYYCARS
GGWLAPFDYWGRGTLVTVSS
9-2 L11 light chain variable region
(SEQ ID NO: 7)
DIVMTQSPDSLAVSLGERATINCKSSQSLFYHSNQKHSLAWYQQKPGQPP
KLLIYGASTRESGVPDRFSGSGSGTDFTLTISSLQAEDVAVYYCQQYYGY
PYTFGGGTKVEIK

Anti PD-L1 antibody 24D5 H12L61:

24D5 H12 heavy chain variable region
(SEQ ID NO: 8)
QVQLVQSGAEVKKPGASVKVSCKASGYTFTSYWMHWVRQAPGQGLEWMGR
ITPSSGFAMYNEKFKNRVTMTRDTSTSTVYMELSSLRSEDTAVYYCARGG
SSYDYFDYWGQGTTVTVSS
24D5 L61 light chain variable region
(SEQ ID NO: 9)
DIVLTQSPASLAVSPGQRATITCRASESVSIHGTHLMHWYQQKPGQPPKL
LIYAASNLESGVPARFSGSGSGTDFTLTINPVEAEDTANYYCQQSFEDPL
TFGQGTKLEIK

Note: The underlined CDRs in the light and heavy chain variable regions derived from the above antibodies 9-2 and 24D5 were the CDRs defined by Kabat numbering criteria.

TABLE 5
Antibody CDRs defined by Kabat numbering criteria

| Anti- | | | | |
|---|---|---|---|---|
| body | Chain | CDR1 | CDR2 | CDR3 |
| 9-2 | Heavy | DGSAYWS | FISRAGSTYNTPSLKG | SGGWLAPFDY |
| H5L11 | chain | (SEQ ID | (SEQ ID NO: 98) | (SEQ ID |
| | HCDR | NO: 97) | | NO: 99) |
| | Light | KSSQSLFY | GASTRES | QQYYGYPYT |
| | chain | HSNQKHSL | (SEQ ID NO: 101) | (SEQ ID |
| | LCDR | A | | NO: 102) |
| | | (SEQ ID | | |
| | | NO: 100) | | |
| 24D5 | Heavy | SYWMH | RITPSSGFAMYNEKFKN | GGSSYDYFDY |
| H12L61 | chain | (SEQ ID | (SEQ ID NO: 104) | (SEQ ID |
| | HCDR | NO: 103) | | NO: 105) |
| | Light | RASESVSI | AASNLES | QQSFEDPLT |
| | chain | HGTHLMH | (SEQ ID NO: 107) | (SEQ ID |
| | LCDR | (SEQ ID | | NO: 108) |
| | | NO: 106) | | |

Primers were desiged; each humanized antibody VH/VK gene fragment was constructed by PCR, and then the homologous recombination with the expression vector pHr (with signal peptide and constant region gene (CH1-FC/CL) fragment) was carried out to construct full-length antibody expressing vector VH-CH1-FC-pHr/VK-CL-pHr.

The sequence of IgG4-S228P heavy chain constant
region was as follows:
(SEQ ID NO: 10)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
The sequence of IgG1 heavy chain constant
region was as follows:
(SEQ ID NO: 11)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
The sequence of the antibody light chain (Kappa
chain) constant region was as follows:
(SEQ ID NO: 12)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

The constructed full-length antibody was as follows:

Anti PD-L1 IgG4 antibody h1830

h1830 heavy chain
(SEQ ID NO: 13)
QVQLQESGPGLVKPSQTLSLTCTVSGGSISDGSAYWSWIRQHPGKGLEYI
GFISRAGSTYNTPSLKGRVTISRDTSKNQFSLKLSSVTAADTAVYYCARS
GGWLAPFDYWGRGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLG
h1830 light chain
(SEQ ID NO: 14)
DIVMTQSPDSLAVSLGERATINCKSSQSLFYHSNQKHSLAWYQQKPGQPP
KLLIYGASTRESGVPDRFSGSGSGTDFTLTISSLQAEDVAVYYCQQYYGY
PYTFGGGTKVEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC

Anti PD-L1 IgG4 antibody h1830G1

h1830G1 heavy chain: (SEQ ID NO: 15)
QVQLQESGPGLVKPSQTLSLTCTVSGGSISDGSAYWSWIRQHPGKGLEYI
GFISRAGSTYNTPSLKGRVTISRDTSKNQFSLKLSSVTAADTAVYYCARS
GGWLAPFDYWGRGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
h1830G1 light chain (same as h1830 light chain,
SEQ ID NO: 14):
DIVMTQSPDSLAVSLGERATINCKSSQSLFYHSNQKHSLAWYQQKPGQPP
KLLIYGASTRESGVPDRFSGSGSGTDFTLTISSLQAEDVAVYYCQQYYGY
PYTFGGGTKVEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC

PD-L1 antibody h1831:

h1831 heavy chain
(SEQ ID NO: 16)
QVQLVQSGAEVKKPGASVKVSCKASGYTFTSYWMHWVRQAPGQGLEWMGR
ITPSSGFAMYNEKFKNRVTMTRDTSTSTVYMELSSLRSEDTAVYYCARGG
SSYDYFDYWGQGTTVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLG
h1831 light chain
(SEQ ID NO: 17)
DIVLTQSPASLAVSPGQRATITCRASESVSIHGTHLMHWYQQKPGQPPKL
LIYAASNLESGVPARFSGSGSGTDFTLTINPVEAEDTANYYCQQSFEDPL
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

Anti PD-L1 IgG1 antibody h1831G1

h1830G1 heavy chain:
(SEQ ID NO: 18)
QVQLVQSGAEVKKPGASVKVSCKASGYTFTSYWMHWVRQAPGQGLEW
MGRITPSSGFAMYNEKFKNRVTMTRDTSTSTVYMELSSLRSEDTAVYYCA
RGGSSYDYFDYWGQGTTVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSP
GK
h1831G1 light chain
(same as h1831 light chain, SEQ ID NO: 17)
DIVLTQSPASLAVSPGQRATITCRASESVSIHGTHLMHWYQQKPGQPPKL
LIYAASNLESGVPARFSGSGSGTDFTLTINPVEAEDTANYYCQQSFEDPL
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC.

### Example 4. Screening and Preparation of SIRPγ Mutants

4.1 Construction of the Affinity Maturation Phage Library of SIRPγ and Screening of the Library

In order to obtain CD47 receptors with high affinity, affinity maturation was performed on the CD47 receptor SIRPγ D1 domain through the phage display platform technology. The affinity maturation phage library directed to the CD47 binding domain was designed and prepared based on the wild-type SIRPγ, and screened for new SIRPγ mutants. The specific sequence of the wild-type SIRPγ D1 domain was as follows:

DNA coding sequence of wild-type SIRPγ peptide:
(SEQ ID NO: 19)
GAGGAGGAGCTACAGATGATTCAGCCTGAGAAGCTCCTGTTGGTCACA
GTTGGAAAGACAGCCACTCTGCACTGCACTGTGACCTCCCTGCTTCCCGT
GGGACCCGTCCTGTGGTTCAGAGGAGTTGGACCAGGCCGGGAATTAATCT
ACAATCAAAAAGAAGGCCACTTCCCCAGGGTAACAACAGTTTCAGACCTC
ACAAAGAGAAACAACATGGACTTTTCCATCCGCATCAGTAGCATCACCCC
AGCAGATGTCGGCACATACTACTGTGTGAAGTTTCGAAAAGGGAGCCCTG
AGAACGTGGAGTTTAAGTCTGGACCAGGCACTGAGATGGCTTTGGGTGCC
AAACCCTCT
Wild-type SIRPγ peptide:
(SEQ ID NO: 20)
EEELQMIQPEKLLLVTVGKTATLHCTVTSLLPVGPVLWFRGVGPGRELIY
NQKEGHFPRVTTVSDLTKRNNMDFSIRISSITPADVGTYYCVKFRKGSPE
NVEFKSGPGTEMALGAKPS.

Construction of the phage library: degenerate primers were designed, and the designed mutant amino acids were introduced into the SIRPγ phage mutant library by PCR, with the size of each library of about 10⁹.

Screening of the SIRPγ phagemutant library: the packaged SIRPγ phage mutant library (1×10¹²-1×10¹³) and 100 μl streptavidin microbeads (Miltenyi Biotec, Auburn, Calif.) were added to 1 ml of phosphate buffered saline (abbreviated as MPBS) comprising 2% skimmed milk and incubated at room temperature for 1 hour, placed on a magnetic stand, and the supernatant was collected. 10 μg/ml biotinylated human CD47-ECD-his protein (purchased from Sino Biological) was added to the supernatant and incubated for 1 hour at room temperature. Then 100 μl of streptavidin-coated magnetic beads (preincubated with 1 ml MPBS) were added and incubated for 1 hour at room temperature. The sample was loaded on the magnetic stand system for sorting, and the supernatant was removed. 1 ml PBST (phosphate buffer comprising 0.1% Tween-20) was added and turned over several times. Fresh washing solution was added after complete removal of supernatant, the step was repeated 11 times to remove unbound mutants, and 0.5 ml elution solution (50 μl 10 mg/ml trypsin stock solution added into 450 μl PBS) was added. The sample was shaken for 15 min at room temperature, placed on a magnetic stand, and the supernatant was transfered into a new EP tube. TG1 was seeded into the 2YT medium and expanded until when the bacteria density was OD600=0.4. 1.75 ml of TG1 (OD600=0.4) was added to each tube, and 250 μl of eluted phage was added, incubated in a 37° C. water bath for 30 min, and spread on plates with gradient dilution for testing the titer. The remaining TG1 solution was centrifuged, spread on plates and incubated overnight at 37° C.

The biotinylated human CD47-ECD-his protein antigen was used for SIRPγ phage mutant library, and after 2-3 rounds of MACS screening (streptavidin magnetic beads, Invitrogen), phage mutant monoclones with higher affinity than the wild-type SIRPγ were finally obtained and subjected to sequencing verification. The sequenced clones were compared and analyzed. After removing redundant sequences, the non-redundant sequences were converted into PDL1-CD47 bifunctional fusion protein for expression in mammalian cell.

4.2 Construction of the Affinity Maturation Yeast Library of SIRPγ and Screening of the Library

In order to obtain CD47 receptors with higher affinity, affinity maturation of the CD47 receptor SIRPγ-D1 domain was carried out by yeast display platform technology. The affinity maturation yeast library directed to the CD47-binding domain was designed and prepared on the basis of SIRPγ-D1, and screened for new SIRPγ mutants.

Construction of the yeast library: degenerate primers were designed, and the designed mutant amino acids were introduced into the SIRPγ mutant library by PCR, with the size of each library of about 10⁹. The constructed yeast library was verified for its diversity by the second-generation sequencing method.

In the first round of screening, about 5×10¹⁰ cells from the SIRPγ mutant library and 10 μg/ml biotinylated human CD47-Fc protein were incubated in 50 ml phosphate buffer (referred to as PBSA) comprising 0.1% bovine serum albumin (BSA) at room temperature for 1 hour. Then, the mixture was washed three times with 0.1% PBSA to remove unbound antibody fragments. Then, 100 μl of streptavidin beads (Milenyi Biotec, Auburn, Calif.) were added to the SIRPγ mutant library that were bound with the biotinylated human CD47-Fc protein, and were loaded on the AutoMACS system for sorting. Library cells with a high affinity for CD47-Fc were collected and then amplified in SDCAA culture medium (20 g dextrose, 6.7 g Difco yeast nitrogen source-free of amino acids, 5 g Bacto casein amino acids, 5.4 g Na₂HPO₄ and 8.56 g NaH₂PO₄.H₂O, dissolved in 1 L of distilled water) at 30° C., 250 rpm for 24 hours. Then, the culture was induced in SGCAA culture medium (20 g galactose, 6.7 g Difco yeast nitrogen source-free of amino acids, 5 g Bacto casein amino acids, 5.4 g Na₂HPO₄ and 8.56 g NaH₂PO₄.H₂O, dissolved in 1 L of distilled water) at 20° C., 250 rpm for 18 hours. The obtained enriched library was subjected to the second round of screening for binding with biotinylated recombinant human CD47-Fc. In order to ensure sufficient diversity of the antibody library for the second and/or third round of screening, a size of the library which is 100-fold of the previous round was used as the number of input cells.

For the third and fourth rounds of screening, the library cells from the previous round were incubated with 1 μg/ml biotinylated human CD47-Fc protein and 10 μg/ml Mouse Anti-cMyc antibody (9E10, sigma) in 0.1% PBSA at room temperature for 1 hour. The mixture was washed three times with 0.1% PBSA to remove unbound antibody fragments. Goat anti-mouse-Alexa488 (A-11001, life technologies) and Strepavidin-PE (S-866, Life technologies) were added and incubated at 4° C. for 1 hour. The mixture was washed three times with 0.1% PBSA to remove unbound antibody fragments. Finally, SIRPγ mutants with high affinity were screened by FACS screening (BD FACSAria™ FUSION).

The biotinylated human CD47-Fc antigen was used for SIRPγ mutant library; 2 to 3 rounds of MACS screening (streptavidin magnetic beads, Invitrogen) and 2 to 3 rounds of FACS screening (BD FACSAria™ FUSION) were performed. About 400 yeast monoclones were then selected for culturing and induced expression. The binding of yeast monoclones to human CD47-Fc antigen was detected by using FACS (BD FACSCanto II), and yeast monoclones with higher affinity than the wild-type SIRPγ were selected and subjected to sequencing verification. The sequenced clones were compared and analyzed. After removing redundant sequences, the non-redundant sequences were converted into PDL1-CD47 bifunctional fusion protein for expression in mammalian cell.

After screening, the SIRPγ peptide variants finally obtained were as follows:

S58
(SEQ ID NO: 21)
EEELQMIQPEKLLLVTVGETATLHCTVTSLLPVGPVLWFRGVGPGRELIY
MSGRGHFPRVTTVSDLTKRNNMDFSIRISSITPADVGTYYCVKFRKGSPE
DVEFKSGPGTEMALGAKPS
S79
(SEQ ID NO: 22)
EEELQMIQPEKLLLVTVGETATLHCTVTSLWPVGPVLWFRGVGPGRELIY
RTGTGRFPRVTTVSDLTKRNNMDFSIRISSITPADVGTYYCVKFRKGSPE
DVEFKSGPGTEVALGAKPS
S15
(SEQ ID NO: 23)
EEELQMIQPEKLLLVTVGETATLHCTVTSLLPVGPVLWFRGVGPGRELIY
VSGRGHFPRVTTVSDLTKRENRDFSIRISSITPADVGTYYCVKFRKGSPE
DVEFKSGPGTEMALGAKPS
S12
(SEQ ID NO: 24)
EEELQMIQPEKLLLVTVGETATLHCTVTSLLPVGPVLWFRGVGPGRELIY
VSGRGHFPRVTTVSDLTKRENKDFSIRISSITPADVGTYYCVKFRKGSPE
DVEFKSGPGTEMALGAKPS
S19
(SEQ ID NO: 25)
EEELQMIQPEKLLLVTVGETATLHCTVTSLLPVGPVLWFRGVGPGRELIY
VSGRGHFPRVTTVSDLTKRNNRDFSIRISSITPADVGTYYCVKFRKGSPE
DVEFKSGPGTEMALGAKPS
S85
(SEQ ID NO: 26)
EEELQMIQPEKLLLVTVGETATLHCTVTSLLPVGPVLWFRGVGPGRELIY
VSGRGHFPRVTTVSDLTKRNNKDFSIRISSITPADVGTYYCVKFRKGSPE
DVEFKSGPGTEMALGAKPS
S37
(SEQ ID NO: 27)
EEELQMIQPEKLLLVTVGETATLHCTVTSLLPVGPVLWFRGVGPGRELIY
MSGRGHFPRVTTVSDLTKRNNKDFSIRISSITPADVGTYYCVKFRKGSPE
DVEFKSGPGTEMALGAKPS
S38
(SEQ ID NO: 28)
EEELQMIQPEKLLLVTVGETATLHCTVTSLLPVGPVLWFRGVGPGRELIY
FSGRGHFPRVTTVSDLTKRNNMDFSIRISSITPADVGTYYCVKFRKGSPE
DVEFKSGPGTEMALGAKPS
S22
(SEQ ID NO: 29)
EEELQMIQPEKLLLVTVGETATLHCTVTSLLPVGPVLWFRGVGPGRELIY
ISGRGHFPRVTTVSDLTKRNNMDFSIRISSITPADVGTYYCVKFRKGSPE
DVEFKSGPGTEMALGAKPS
S29
(SEQ ID NO: 30)
EEELQMIQPEKLLLVTVGETATLHCTVTSLLPVGPVLWFRGVGPGRELIY
LSGRGHFPRVTTVSDLTKRNNMDFSIRISSITPADVGTYYCVKFRKGSPE
DVEFKSGPGTEMALGAKPS
S34
(SEQ ID NO: 31)
EEELQMIQPEKLLLVTVGETATLHCTVTSLLPVGPVLWFRGVGPGRELIY
RSGRGHFPRVTTVSDLTKRNNMDFSIRISSITPADVGTYYCVKFRKGSPE
DVEFKSGPGTEMALGAKPS
S41
(SEQ ID NO: 32)
EEELQMIQPEKLLLVTVGETATLHCTVTSLLPVGPVLWFRGVGPGRELIY
VSGRGHFPRVTTVSDLTKRNNMDFSIRISSITPADVGTYYCVKFRKGSPE
DVEFKSGPGTEMALGAKPS
S42
(SEQ ID NO: 33)
EEELQMIQPEKLLLVTVGETATLHCTVTSLLPVGPVLWFRGVGPGRELIY
MSGRGHFPRVTTVSDLTKRNNIDFSIRISSITPADVGTYYCVKFRKGSPE
DVEFKSGPGTEMALGAKPS
S43
(SEQ ID NO: 34)
EEELQMIQPEKLLLVTVGETATLHCTVTSLLPVGPVLWFRGVGPGRELIY
MSGRGHFPRVTTVSDLTKRNNRDFSIRISSITPADVGTYYCVKFRKGSPE
DVEFKSGPGTEMALGAKPS
S44
(SEQ ID NO: 35)
EEELQMIQPEKLLLVTVGETATLHCTVTSLLPVGPVLWFRGVGPGRELIY
MSGRGHFPRVTTVSDLTKRNNVDFSIRISSITPADVGTYYCVKFRKGSPE
DVEFKSGPGTEMALGAKPS
S45
(SEQ ID NO: 36)
EEELQMIQPEKLLLVTVGETATLHCTVTSLLPVGPVLWFRGVGPGRELIY
MSGRGHFPRVTTVSDLTKRDNMDFSIRISSITPADVGTYYCVKFRKGSPE
DVEFKSGPGTEMALGAKPS
S46
(SEQ ID NO: 37)
EEELQMIQPEKLLLVTVGETATLHCTVTSLLPVGPVLWFRGVGPGRELIY
MSGRGHFPRVTTVSDLTKRENKDFSIRISSITPADVGTYYCVKFRKGSPE
DVEFKSGPGTEMALGAKPS
S47
(SEQ ID NO: 38)
EEELQMIQPEKLLLVTVGETATLHCTVTSLLPVGPVLWFRGVGPGRELIY
MSGRGHFPRVTTVSDLTKRENKDFSIRISSITPADVGTYYCVKFRKGSPE
DVEFKSGPGTEVALGAKPS
S48
(SEQ ID NO: 39)
EEELQMIQPEKLLLVTVGETATLHCTVTSLLPVGPVLWFRGVGPGRELIY
RSGRGHFPRVTTVSDLTKRNNKDFSIRISSITPADVGTYYCVKFRKGSPE
DVEFKSGPGTEMALGAKPS
S49
(SEQ ID NO: 40)
EEELQMIQPEKLLLVTVGETATLHCTVTSLLPVGPVLWFRGVGPGRELIY
RSGRGHFPRVTTVSDLTKRENKDFSIRISSITPADVGTYYCVKFRKGSPE
DVEFKSGPGTEMALGAKPS.

### Example 5. Construction and Expression of PD-L1-CD47 Bifunctional Fusion Protein

The obtained anti PD-L1 antibodies were linked to SIRPγ to form fusion proteins, and the PD-L1-CD47 bifunctional fusion proteins were obtained by expression and purification by conventional methods.

TABLE 6
Sequences of PD-L1-CD47 bifunctional fusion proteins

| | Heavy chain + linker | |
|---|---|---|
| Protein | (if present) + SIRP γ variant | Light chain |
| h1830-S58 | QVQLQESGPGLVKPSQTLSLTCTVSGGSISDGSA | DIVMTQSPDS |
| | YWSWIRQHPGKGLEYIGFISRAGSTYNTPSLKGR | NCKSSQSLFY |
| | VTISRDTSKNQFSLKLSSVTAADTAVYYCARSGG | HSNQKHSLA |
| | WLAPFDYWGRGTLVTVSSASTKGPSVFPLAPCS | WYQQKPGQP |
| | RSTSESTAALGCLVKDYFPEPVTVSWNSGALTSG | PKLLIYGAST |
| | VHTFPAVLQSSGLYSLSSVVTVPSSSLGTKTYTCN | RESGVPDRFS |
| | VDHKPSNTKVDKRVESKYGPPCPPCPAPEFLGGP | GSGSGTDFTL |
| | SVFLFPPKPKDTLMISRTPEVTCVVVDVSQEDPE | TISSLQAEDVA |
| | VQFNWYVDGVEVHNAKTKPREEQFNSTYRVVS | VYYCQQYYG |
| | VLTVLHQDWLNGKEYKCKVSNKGLPSSIEKTISK | YPYTFGGGTK |
| | AKGQPREPQVYTLPPSQEEMTKNQVSLTCLVKG | VEIKRTVAAPS |
| | FYPSDIAVEWESNGQPENNYKTTPPVLDSDGSFF | VFIFPPSDEQL |
| | LYSRLTVDKSRWQEGNVFSCSVMHEALHNHYT | KSGTASVVCL |
| | QKSLSLSLGGGGGSGGGGSGGGGGGEEELQMIQ | LNNFYPREAK |
| | PEKLLLVTVGETATLHCTVTSLLPVGPVLWFRGV | VQWKVDNAL |
| | GPGRELIYMSGRGHFPRVTTVSDLTKRNNMDFSI | QSGNSQESVT |
| | RISSITPADVGTYYCVKFRKGSPEDVEFKSGPGTE | EQDSKDSTYS |
| | MALGAKPS | LSSTLTLSKA |
| | (SEQ ID NO: 41) | DYEKHKVYA |
| | | CEVTHQGLSS |
| | | LAVSLGERATI |
| | | PVTKSFNRGEC |
| | | (SEQ ID NO: 14, |
| | | the same as |
| | | that of h1830 |
| | | light chain) |
| h1830-S15 | QVQLQESGPGLVKPSQTLSLTCTVSGGSISDGSA | |
| | YWSWIRQHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTSG | |
| | VHTFPAVLQSSGLYSLSSVVTVPSSSLGTKTYTCN | |
| | VDHKPSNTKVDKRVESKYGPPCPPCPAPEFLGGP | |
| | SVFLFPPKPKDTLMISRTPEVTCVVVDVSQEDPE | |
| | VQFNWYVDGVEVHNAKTKPREEQFNSTYRVVS | |
| | VLTVLHQDWLNGKEYKCKVSNKGLPSSIEKTISK | |
| | AKGQPREPQVYTLPPSQEEMTKNQVSLTCLVKG | |
| | FYPSDIAVEWESNGQPENNYKTTPPVLDSDGSFF | |
| | LYSRLTVDKSRWQEGNVFSCSVMHEALHNHYT | |
| | QKSLSLSLGGGGGSGGGGSGGGGGGEEELQMIQ | |
| | PEKLLLVTVGETATLHCTVTSLLPVGPVLWFRGV | |
| | GPGRELIYVSGRGHFPRVTTVSDLTKRENRDFSIR | |
| | ISSITPADVGTYYCVKFRKGSPEDVEFKSGPGTE | |
| | MALGAKPS | |
| | (SEQ ID NO: 42) | |
| h1830-S12 | QVQLQESGPGLVKPSQTLSLTCTVSGGSISDGSA | |
| | YWSWIRQHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTSG | |
| | VHTFPAVLQSSGLYSLSSVVTVPSSSLGTKTYTCN | |
| | VDHKPSNTKVDKRVESKYGPPCPPCPAPEFLGGP | |
| | SVFLFPPKPKDTLMISRTPEVTCVVVDVSQEDPE | |
| | VQFNWYVDGVEVHNAKTKPREEQFNSTYRVVS | |
| | VLTVLHQDWLNGKEYKCKVSNKGLPSSIEKTISK | |
| | AKGQPREPQVYTLPPSQEEMTKNQVSLTCLVKG | |
| | FYPSDIAVEWESNGQPENNYKTTPPVLDSDGSFF | |
| | LYSRLTVDKSRWQEGNVFSCSVMHEALHNHYT | |
| | QKSLSLSLGGGGGSGGGGSGGGGGGEEELQMIQ | |
| | PEKLLLVTVGETATLHCTVTSLLPVGPVLWFRGV | |
| | GPGRELIYVSGRGHFPRVTTVSDLTKRENKDFSIR | |
| | ISSITPADVGTYYCVKFRKGSPEDVEFKSGPGTE | |
| | MALGAKPS | |
| | (SEQ ID NO: 43) | |
| h1830-S19 | QVQLQESGPGLVKPSQTLSLTCTVSGGSISDGSA | |
| | YWSWIRQHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTSG | |
| | VHTFPAVLQSSGLYSLSSVVTVPSSSLGTKTYTCN | |
| | VDHKPSNTKVDKRVESKYGPPCPPCPAPEFLGGP | |
| | SVFLFPPKPKDTLMISRTPEVTCVVVDVSQEDPE | |
| | VQFNWYVDGVEVHNAKTKPREEQFNSTYRVVS | |
| | VLTVLHQDWLNGKEYKCKVSNKGLPSSIEKTISK | |
| | AKGQPREPQVYTLPPSQEEMTKNQVSLTCLVKG | |
| | FYPSDIAVEWESNGQPENNYKTTPPVLDSDGSFF | |
| | LYSRLTVDKSRWQEGNVFSCSVMHEALHNHYT | |
| | QKSLSLSLGGGGGSGGGGSGGGGGGEEELQMIQ | |
| | PEKLLLVTVGETATLHCTVTSLLPVGPVLWFRGV | |
| | GPGRELIYVSGRGHFPRVTTVSDLTKRNNRDFSI | |
| | RISSITPADVGTYYCVKFRKGSPEDVEFKSGPGTE | |
| | MALGAKPS | |
| | (SEQ ID NO: 44) | |
| h1830-S85 | QVQLQESGPGLVKPSQTLSLTCTVSGGSISDGSA | |
| | YWSWIRQHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTSG | |
| | VHTFPAVLQSSGLYSLSSVVTVPSSSLGTKTYTCN | |
| | VDHKPSNTKVDKRVESKYGPPCPPCPAPEFLGGP | |
| | SVFLFPPKPKDTLMISRTPEVTCVVVDVSQEDPE | |
| | VQFNWYVDGVEVHNAKTKPREEQFNSTYRVVS | |
| | VLTVLHQDWLNGKEYKCKVSNKGLPSSIEKTISK | |
| | AKGQPREPQVYTLPPSQEEMTKNQVSLTCLVKG | |
| | FYPSDIAVEWESNGQPENNYKTTPPVLDSDGSFF | |
| | LYSRLTVDKSRWQEGNVFSCSVMHEALHNHYT | |
| | QKSLSLSLGGGGGSGGGGSGGGGGGEEELQMIQ | |
| | PEKLLLVTVGETATLHCTVTSLLPVGPVLWFRGV | |
| | GPGRELIYVSGRGHFPRVTTVSDLTKRNNKDFSI | |
| | RISSITPADVGTYYCVKFRKGSPEDVEFKSGPGTE | |
| | MALGAKPS | |
| | (SEQ ID NO: 45) | |
| h1830-S37 | QVQLQESGPGLVKPSQTLSLTCTVSGGSISDGSA | |
| | YWSWIRQHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTSG | |
| | VHTFPAVLQSSGLYSLSSVVTVPSSSLGTKTYTCN | |
| | VDHKPSNTKVDKRVESKYGPPCPPCPAPEFLGGP | |
| | SVFLFPPKPKDTLMISRTPEVTCVVVDVSQEDPE | |
| | VQFNWYVDGVEVHNAKTKPREEQFNSTYRVVS | |
| | VLTVLHQDWLNGKEYKCKVSNKGLPSSIEKTISK | |
| | AKGQPREPQVYTLPPSQEEMTKNQVSLTCLVKG | |
| | FYPSDIAVEWESNGQPENNYKTTPPVLDSDGSFF | |
| | LYSRLTVDKSRWQEGNVFSCSVMHEALHNHYT | |
| | QKSLSLSLGGGGGSGGGGSGGGGGGEEELQMIQ | |
| | PEKLLLVTVGETATLHCTVTSLLPVGPVLWFRGV | |
| | GPGRELIYMSGRGHFPRVTTVSDLTKRNNKDFSI | |
| | RISSITPADVGTYYCVKFRKGSPEDVEFKSGPGTE | |
| | MALGAKPS | |
| | (SEQ ID NO: 46) | |
| h1830-19- | QVQLQESGPGLVKPSQTLSLTCTVSGGSISDGSA | |
| S79 | YWSWIROHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTS | |
| | GVHTFPAVLQSSGLYSLSSVVTVPSSSLGTKTYT | |
| | CNVDHKPSNTKVDKRVESKYGPPCPRCPAPEFL | |
| | GGPSVFLFPPKPKDTLMISRTPEVTCVVVDVSQE | |
| | DPEVQFNWYVDGVEVHNAKTKPREEQFNSTYR | |
| | VVSVLTVLHQDWLNGKEYKCKVSNKGLPSSIEK | |
| | TISKAKGQPREPQVYTLPPSQEEMTKNQVSLTCL | |
| | VKGFYPSDIAVEWESNGQPENNYKTTPPVLDSD | |
| | GSFFLYSRLTVDKSRWQEGNVFSCSVMHEALHN | |
| | HYTQKSLSLSLGGGGGSGGGGSGGGGSGGGGE | |
| | EELQMIQPEKLLLVTVGETATLHCTVTSLWPVGP | |
| | VLWFRGVGPGRELIYRTGTGRFPRVTTVSDLTKR | |
| | NNMDFSIRISSITPADVGTYYCVKFRKGSPEDVEF | |
| | KSGPGTEVALGAKPS | |
| | (SEQ ID NO: 47) | |
| h1830-S79 | QVQLQESGPGLVKPSQTLSLTCTVSGGSISDGSA | |
| | YWSWIROHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTS | |
| | GVHTFPAVLQSSGLYSLSSVVTVPSSSLGTKTYT | |
| | CNVDHKPSNTKVDKRVESKYGPPCPRCPAPEFL | |
| | GGPSVFLFPPKPKDTLMISRTPEVTCVVVDVSQE | |
| | DPEVQFNWYVDGVEVHNAKTKPREEQFNSTYR | |
| | VVSVLTVLHQDWLNGKEYKCKVSNKGLPSSIEK | |
| | TISKAKGQPREPQVYTLPPSQEEMTKNQVSLTCL | |
| | VKGFYPSDIAVEWESNGQPENNYKTTPPVLDSD | |
| | GSFFLYSRLTVDKSRWQEGNVFSCSVMHEALHN | |
| | HYTQKSLSLSLGGGGGSGGGGSGGGGGGEEELQ | |
| | MIQPEKLLLVTVGETATLHCTVTSLWPVGPVLWF | |
| | RGVGPGRELIYRTGTGRFPRVTTVSDLTKRNNM | |
| | DFSIRISSITPADVGTYYCVKFRKGSPEDVEFKSG | |
| | PGTEVALGAKPS | |
| | (SEQ ID NO: 48) | |
| h1830G1- | QVQLQESGPGLVKPSQTLSLTCTVSGGSISDGSA | |
| 19-S79 | YWSWIRQHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPSS | |
| | KSTSGGTAALGCLVKDYFPEPVTVSWNSGALTS | |
| | GVHTFPAVLQSSGLYSLSSVVTVPSSSLGTQTYIC | |
| | NVNHKPSNTKVDKKVEPKSCDKTHTCPPCPAPE | |
| | LLGGPSVFLFPPKPKDTLMISRTPEVTCVVVDVS | |
| | HEDPEVKFNWYVDGVEVHNAKTKPREEQYNST | |
| | YRVVSVLTVLHQDWLNGKEYKCKVSNKALPAPI | |
| | EKTISKAKGQPREPQVYTLPPSRDELTKNQVSLT | |
| | CLVKGFYPSDIAVEWESNGQPENNYKTTPPVLDS | |
| | DGSFFLYSKLTVDKSRWQQGNVFSCSVMHEALH | |
| | NHYTQKSLSLSPGKGGGGGSGGGGSGGGGSGG | |
| | GGEEELQMIQPEKLLLVTVGETATLHCTVTSLWP | |
| | VGPVLWFRGVGPGRELIYRTGTGRFPRVTTVSDL | |
| | TKRNNMDFSIRISSITPADVGTYYCVKFRKGSPE | |
| | DVEFKSGPGTEVALGAKPS | |
| | (SEQ ID NO: 49) | |
| h1830-S38 | QVQLQESGPGLVKPSQTLSLTCTVSGGSISDGSA | |
| | YWSWIRQHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTSG | |
| | VHTFPAVLQSSGLYSLSSVVTVPSSSLGTKTYTCN | |
| | VDHKPSNTKVDKRVESKYGPPCPPCPAPEFLGGP | |
| | SVFLFPPKPKDTLMISRTPEVTCVVVDVSQEDPE | |
| | VQFNWYVDGVEVHNAKTKPREEQFNSTYRVVS | |
| | VLTVLHQDWLNGKEYKCKVSNKGLPSSIEKTISK | |
| | AKGQPREPQVYTLPPSQEEMTKNQVSLTCLVKG | |
| | FYPSDIAVEWESNGQPENNYKTTPPVLDSDGSFF | |
| | LYSRLTVDKSRWQEGNVFSCSVMHEALHNHYT | |
| | QKSLSLSLGGGGGSGGGGSGGGGGGEEELQMIQ | |
| | PEKLLLVTVGETATLHCTVTSLLPVGPVLWFRGV | |
| | GPGRELIYFSGRGHFPRVTTVSDLTKRNNMDFSI | |
| | RISSITPADVGTYYCVKFRKGSPEDVEFKSGPGTE | |
| | MALGAKPS | |
| | (SEQ ID NO: 50) | |
| h1830-S22 | QVQLQESGPGLVKPSQTLSLTCTVSGGSISDGSA | |
| | YWSWIRQHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTSG | |
| | VHTFPAVLQSSGLYSLSSVVTVPSSSLGTKTYTCN | |
| | VDHKPSNTKVDKRVESKYGPPCPPCPAPEFLGGP | |
| | SVFLFPPKPKDTLMISRTPEVTCVVVDVSQEDPE | |
| | VQFNWYVDGVEVHNAKTKPREEQFNSTYRVVS | |
| | VLTVLHQDWLNGKEYKCKVSNKGLPSSIEKTISK | |
| | AKGQPREPQVYTLPPSQEEMTKNQVSLTCLVKG | |
| | FYPSDIAVEWESNGQPENNYKTTPPVLDSDGSFF | |
| | LYSRLTVDKSRWQEGNVFSCSVMHEALHNHYT | |
| | QKSLSLSLGGGGGSGGGGSGGGGGGEEELQMIQ | |
| | PEKLLLVTVGETATLHCTVTSLLPVGPVLWFRGV | |
| | GPGRELIYISGRGHFPRVTTVSDLTKRNNMDFSIR | |
| | ISSITPADVGTYYCVKFRKGSPEDVEFKSGPGTE | |
| | MALGAKPS | |
| | (SEQ ID NO: 51) | |
| h1830-S29 | QVQLQESGPGLVKPSQTLSLTCTVSGGSISDGSA | |
| | YWSWIRQHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTSG | |
| | VHTFPAVLQSSGLYSLSSVVTVPSSSLGTKTYTCN | |
| | VDHKPSNTKVDKRVESKYGPPCPPCPAPEFLGGP | |
| | SVFLFPPKPKDTLMISRTPEVTCVVVDVSQEDPE | |
| | VQFNWYVDGVEVHNAKTKPREEQFNSTYRVVS | |
| | VLTVLHQDWLNGKEYKCKVSNKGLPSSIEKTISK | |
| | AKGQPREPQVYTLPPSQEEMTKNQVSLTCLVKG | |
| | FYPSDIAVEWESNGQPENNYKTTPPVLDSDGSFF | |
| | LYSRLTVDKSRWQEGNVFSCSVMHEALHNHYT | |
| | QKSLSLSLGGGGGSGGGGSGGGGGGEEELQMIQ | |
| | PEKLLLVTVGETATLHCTVTSLLPVGPVLWFRGV | |
| | GPGRELIYLSGRGHFPRVTTVSDLTKRNNMDFSI | |
| | RISSITPADVGTYYCVKFRKGSPEDVEFKSGPGTE | |
| | MALGAKPS | |
| | (SEQ ID NO: 52) | |
| h1830-S34 | QVQLQESGPGLVKPSQTLSLTCTVSGGSISDGSA | |
| | YWSWIRQHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTSG | |
| | VHTFPAVLQSSGLYSLSSVVTVPSSSLGTKTYTCN | |
| | VDHKPSNTKVDKRVESKYGPPCPPCPAPEFLGGP | |
| | SVFLFPPKPKDTLMISRTPEVTCVVVDVSQEDPE | |
| | VQFNWYVDGVEVHNAKTKPREEQFNSTYRVVS | |
| | VLTVLHQDWLNGKEYKCKVSNKGLPSSIEKTISK | |
| | AKGQPREPQVYTLPPSQEEMTKNQVSLTCLVKG | |
| | FYPSDIAVEWESNGQPENNYKTTPPVLDSDGSFF | |
| | LYSRLTVDKSRWQEGNVFSCSVMHEALHNHYT | |
| | QKSLSLSLGGGGGSGGGGSGGGGGGEEELQMIQ | |
| | PEKLLLVTVGETATLHCTVTSLLPVGPVLWFRGV | |
| | GPGRELIYRSGRGHFPRVTTVSDLTKRNNMDFSI | |
| | RISSITPADVGTYYCVKFRKGSPEDVEFKSGPGTE | |
| | MALGAKPS | |
| | (SEQ ID NO: 53) | |
| h1830-S41 | QVQLQESGPGLVKPSQTLSLTCTVSGGSISDGSA | |
| | YWSWIRQHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTSG | |
| | VHTFPAVLQSSGLYSLSSVVTVPSSSLGTKTYTCN | |
| | VDHKPSNTKVDKRVESKYGPPCPPCPAPEFLGGP | |
| | SVFLFPPKPKDTLMISRTPEVTCVVVDVSQEDPE | |
| | VQFNWYVDGVEVHNAKTKPREEQFNSTYRVVS | |
| | VLTVLHQDWLNGKEYKCKVSNKGLPSSIEKTISK | |
| | AKGQPREPQVYTLPPSQEEMTKNQVSLTCLVKG | |
| | FYPSDIAVEWESNGQPENNYKTTPPVLDSDGSFF | |
| | LYSRLTVDKSRWQEGNVFSCSVMHEALHNHYT | |
| | QKSLSLSLGGGGGSGGGGSGGGGGGEEELQMIQ | |
| | PEKLLLVTVGETATLHCTVTSLLPVGPVLWFRGV | |
| | GPGRELIYVSGRGHFPRVTTVSDLTKRNNMDFSI | |
| | RISSITPADVGTYYCVKFRKGSPEDVEFKSGPGTE | |
| | MALGAKPS | |
| | (SEQ ID NO: 54) | |
| h1830-S42 | QVQLQESGPGLVKPSQTLSLTCTVSGGSISDGSA | |
| | YWSWIRQHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTSG | |
| | VHTFPAVLQSSGLYSLSSVVTVPSSSLGTKTYTCN | |
| | VDHKPSNTKVDKRVESKYGPPCPPCPAPEFLGGP | |
| | SVFLFPPKPKDTLMISRTPEVTCVVVDVSQEDPE | |
| | VQFNWYVDGVEVHNAKTKPREEQFNSTYRVVS | |
| | VLTVLHQDWLNGKEYKCKVSNKGLPSSIEKTISK | |
| | AKGQPREPQVYTLPPSQEEMTKNQVSLTCLVKG | |
| | FYPSDIAVEWESNGQPENNYKTTPPVLDSDGSFF | |
| | LYSRLTVDKSRWQEGNVFSCSVMHEALHNHYT | |
| | QKSLSLSLGGGGGSGGGGSGGGGGGEEELQMIQ | |
| | PEKLLLVTVGETATLHCTVTSLLPVGPVLWFRGV | |
| | GPGRELIYMSGRGHFPRVTTVSDLTKRNNIDFSIR | |
| | ISSITPADVGTYYCVKFRKGSPEDVEFKSGPGTE | |
| | MALGAKPS | |
| | (SEQ ID NO: 55) | |
| h1830-S43 | QVQLQESGPGLVKPSQTLSLTCTVSGGSISDGSA | |
| | YWSWIRQHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTSG | |
| | VHTFPAVLQSSGLYSLSSVVTVPSSSLGTKTYTCN | |
| | VDHKPSNTKVDKRVESKYGPPCPPCPAPEFLGGP | |
| | SVFLFPPKPKDTLMISRTPEVTCVVVDVSQEDPE | |
| | VQFNWYVDGVEVHNAKTKPREEQFNSTYRVVS | |
| | VLTVLHQDWLNGKEYKCKVSNKGLPSSIEKTISK | |
| | AKGQPREPQVYTLPPSQEEMTKNQVSLTCLVKG | |
| | FYPSDIAVEWESNGQPENNYKTTPPVLDSDGSFF | |
| | LYSRLTVDKSRWQEGNVFSCSVMHEALHNHYT | |
| | QKSLSLSLGGGGGSGGGGSGGGGGGEEELQMIQ | |
| | PEKLLLVTVGETATLHCTVTSLLPVGPVLWFRGV | |
| | GPGRELIYMSGRGHFPRVTTVSDLTKRNNRDFSI | |
| | RISSITPADVGTYYCVKFRKGSPEDVEFKSGPGTE | |
| | MALGAKPS | |
| | (SEQ ID NO: 56) | |
| h1830-S44 | QVQLQESGPGLVKPSQTLSLTCTVSGGSISDGSA | |
| | YWSWIRQHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTSG | |
| | VHTFPAVLQSSGLYSLSSVVTVPSSSLGTKTYTCN | |
| | VDHKPSNTKVDKRVESKYGPPCPPCPAPEFLGGP | |
| | SVFLFPPKPKDTLMISRTPEVTCVVVDVSQEDPE | |
| | VQFNWYVDGVEVHNAKTKPREEQFNSTYRVVS | |
| | VLTVLHQDWLNGKEYKCKVSNKGLPSSIEKTISK | |
| | AKGQPREPQVYTLPPSQEEMTKNQVSLTCLVKG | |
| | FYPSDIAVEWESNGQPENNYKTTPPVLDSDGSFF | |
| | LYSRLTVDKSRWQEGNVFSCSVMHEALHNHYT | |
| | QKSLSLSLGGGGGSGGGGSGGGGGGEEELQMIQ | |
| | PEKLLLVTVGETATLHCTVTSLLPVGPVLWFRGV | |
| | GPGRELIYMSGRGHFPRVTTVSDLTKRNNVDFSI | |
| | RISSITPADVGTYYCVKFRKGSPEDVEFKSGPGTE | |
| | MALGAKPS | |
| | (SEQ ID NO: 57) | |
| h1830-S45 | QVQLQESGPGLVKPSQTLSLTCTVSGGSISDGSA | |
| | YWSWIRQHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTSG | |
| | VHTFPAVLQSSGLYSLSSVVTVPSSSLGTKTYTCN | |
| | VDHKPSNTKVDKRVESKYGPPCPPCPAPEFLGGP | |
| | SVFLFPPKPKDTLMISRTPEVTCVVVDVSQEDPE | |
| | VQFNWYVDGVEVHNAKTKPREEQFNSTYRVVS | |
| | VLTVLHQDWLNGKEYKCKVSNKGLPSSIEKTISK | |
| | AKGQPREPQVYTLPPSQEEMTKNQVSLTCLVKG | |
| | FYPSDIAVEWESNGQPENNYKTTPPVLDSDGSFF | |
| | LYSRLTVDKSRWQEGNVFSCSVMHEALHNHYT | |
| | QKSLSLSLGGGGGSGGGGSGGGGGGEEELQMIQ | |
| | PEKLLLVTVGETATLHCTVTSLLPVGPVLWFRGV | |
| | GPGRELIYMSGRGHFPRVTTVSDLTKRDNMDFSI | |
| | RISSITPADVGTYYCVKFRKGSPEDVEFKSGPGTE | |
| | MALGAKPS | |
| | (SEQ ID NO: 58) | |
| h1830-S46 | QVQLQESGPGLVKPSQTLSLTCTVSGGSISDGSA | |
| | YWSWIRQHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTSG | |
| | VHTFPAVLQSSGLYSLSSVVTVPSSSLGTKTYTCN | |
| | VDHKPSNTKVDKRVESKYGPPCPPCPAPEFLGGP | |
| | SVFLFPPKPKDTLMISRTPEVTCVVVDVSQEDPE | |
| | VQFNWYVDGVEVHNAKTKPREEQFNSTYRVVS | |
| | VLTVLHQDWLNGKEYKCKVSNKGLPSSIEKTISK | |
| | AKGQPREPQVYTLPPSQEEMTKNQVSLTCLVKG | |
| | FYPSDIAVEWESNGQPENNYKTTPPVLDSDGSFF | |
| | LYSRLTVDKSRWQEGNVFSCSVMHEALHNHYT | |
| | QKSLSLSLGGGGGSGGGGSGGGGGGEEELQMIQ | |
| | PEKLLLVTVGETATLHCTVTSLLPVGPVLWFRGV | |
| | GPGRELIYMSGRGHFPRVTTVSDLTKRENKDFSI | |
| | RISSITPADVGTYYCVKFRKGSPEDVEFKSGPGTE | |
| | MALGAKPS | |
| | (SEQ ID NO: 59) | |
| h1830-S47 | QVQLQESGPGLVKPSQTLSLTCTVSGGSISDGSA | |
| | YWSWIRQHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTSG | |
| | VHTFPAVLQSSGLYSLSSVVTVPSSSLGTKTYTCN | |
| | VDHKPSNTKVDKRVESKYGPPCPPCPAPEFLGGP | |
| | SVFLFPPKPKDTLMISRTPEVTCVVVDVSQEDPE | |
| | VQFNWYVDGVEVHNAKTKPREEQFNSTYRVVS | |
| | VLTVLHQDWLNGKEYKCKVSNKGLPSSIEKTISK | |
| | AKGQPREPQVYTLPPSQEEMTKNQVSLTCLVKG | |
| | FYPSDIAVEWESNGQPENNYKTTPPVLDSDGSFF | |
| | LYSRLTVDKSRWQEGNVFSCSVMHEALHNHYT | |
| | QKSLSLSLGGGGGSGGGGSGGGGGGEEELQMIQ | |
| | PEKLLLVTVGETATLHCTVTSLLPVGPVLWFRGV | |
| | GPGRELIYMSGRGHFPRVTTVSDLTKRENKDFSI | |
| | RISSITPADVGTYYCVKFRKGSPEDVEFKSGPGTE | |
| | VALGAKPS | |
| | (SEQ ID NO: 60) | |
| h1830-S48 | QVQLQESGPGLVKPSQTLSLTCTVSGGSISDGSA | |
| | YWSWIRQHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTSG | |
| | VHTFPAVLQSSGLYSLSSVVTVPSSSLGTKTYTCN | |
| | VDHKPSNTKVDKRVESKYGPPCPPCPAPEFLGGP | |
| | SVFLFPPKPKDTLMISRTPEVTCVVVDVSQEDPE | |
| | VQFNWYVDGVEVHNAKTKPREEQFNSTYRVVS | |
| | VLTVLHQDWLNGKEYKCKVSNKGLPSSIEKTISK | |
| | AKGQPREPQVYTLPPSQEEMTKNQVSLTCLVKG | |
| | FYPSDIAVEWESNGQPENNYKTTPPVLDSDGSFF | |
| | LYSRLTVDKSRWQEGNVFSCSVMHEALHNHYT | |
| | QKSLSLSLGGGGGSGGGGSGGGGGGEEELQMIQ | |
| | PEKLLLVTVGETATLHCTVTSLLPVGPVLWFRGV | |
| | GPGRELIYRSGRGHFPRVTTVSDLTKRNNKDFSI | |
| | RISSITPADVGTYYCVKFRKGSPEDVEFKSGPGTE | |
| | MALGAKPS | |
| | (SEQ ID NO: 61) | |
| h1830-S49 | QVQLQESGPGLVKPSQTLSLTCTVSGGSISDGSA | |
| | YWSWIRQHPGKGLEYIGFISRAGSTYNTPSLKGR | |
| | VTISRDTSKNQFSLKLSSVTAADTAVYYCARSGG | |
| | WLAPFDYWGRGTLVTVSSASTKGPSVFPLAPCS | |
| | RSTSESTAALGCLVKDYFPEPVTVSWNSGALTSG | |
| | VHTFPAVLQSSGLYSLSSVVTVPSSSLGTKTYTCN | |
| | VDHKPSNTKVDKRVESKYGPPCPPCPAPEFLGGP | |
| | SVFLFPPKPKDTLMISRTPEVTCVVVDVSQEDPE | |
| | VQFNWYVDGVEVHNAKTKPREEQFNSTYRVVS | |
| | VLTVLHQDWLNGKEYKCKVSNKGLPSSIEKTISK | |
| | AKGQPREPQVYTLPPSQEEMTKNQVSLTCLVKG | |
| | FYPSDIAVEWESNGQPENNYKTTPPVLDSDGSFF | |
| | LYSRLTVDKSRWQEGNVFSCSVMHEALHNHYT | |
| | QKSLSLSLGGGGGSGGGGSGGGGGGEEELQMIQ | |
| | PEKLLLVTVGETATLHCTVTSLLPVGPVLWFRGV | |
| | GPGRELIYRSGRGHFPRVTTVSDLTKRENKDFSIR | |
| | ISSITPADVGTYYCVKFRKGSPEDVEFKSGPGTE | |
| | MALGAKPS | |
| | (SEQ ID NO: 62) | |
| h1831-19- | QVQLVQSGAEVKKPGASVKVSCKASGYTFTSY | DIVLTQSPASL |
| S58 | WMHWVRQAPGQGLEWMGRITPSSGFAMYNEK | AVSPGQRATIT |
| | FKNRVTMTRDTSTSTVYMELSSLRSEDTAVYYC | CRASESVSIH |
| | ARGGSSYDYFDYWGQGTTVTVSSASTKGPSVFP | GTHLMHWYQ |
| | LAPCSRSTSESTAALGCLVKDYFPEPVTVSWNSG | QKPGQPPKLL |
| | ALTSGVHTFPAVLQSSGLYSLSSVVTVPSSSLGTK | IYAASNLESG |
| | TYTCNVDHKPSNTKVDKRVESKYGPPCPPCPAPE | VPARFSGSGS |
| | FLGGPSVFLFPPKPKDTLMISRTPEVTCVVVDVS | GTDFTLTINPV |
| | QEDPEVQFNWYVDGVEVHNAKTKPREEQFNST | EAEDTANYYC |
| | YRVVSVLTVLHQDWLNGKEYKCKVSNKGLPSSI | QQSFEDPLTF |
| | EKTISKAKGQPREPQVYTLPPSQEEMTKNQVSLT | GQGTKLEIKR |
| | CLVKGFYPSDIAVEWESNGQPENNYKTTPPVLDS | TVAAPSVFIFP |
| | DGSFFLYSRLTVDKSRWQEGNVFSCSVMHEALH | PSDEQLKSGT |
| | NHYTQKSLSLSLGGGGGSGGGGSGGGGSGGGG | ASVVCLLNNF |
| | EEELQMIQPEKLLLVTVGETATLHCTVTSLLPVG | YPREAKVQW |
| | PVLWFRGVGPGRELIYMSGRGHFPRVTTVSDLT | KVDNALQSG |
| | KRNNMDFSIRISSITPADVGTYYCVKFRKGSPED | NSQESVTEQD |
| | VEFKSGPGTEMALGAKPS | SKDSTYSLSS |
| | (SEQ ID NO: 63) | TLTLSKADYE |
| | | KHKVYACEV |
| | | THQGLSSPVT |
| | | KSFNRGEC |
| | | (SEQ ID |
| | | NO: 17, the |
| | | same as that of |
| | | h1831 light |
| | | chain) |
| h1831-S15 | QVQLVQSGAEVKKPGASVKVSCKASGYTFTSY | |
| | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPRCPA | |
| | PEFLGGPSVFLFPPKPKDTLMISRTPEVTCVVVD | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPPV | |
| | LDSDGSFFLYSRLTVDKSRWQEGNVFSCSVMHE | |
| | ALHNHYTQKSLSLSLGGGGGSGGGGSGGGGGG | |
| | EEELQMIQPEKLLLVTVGETATLHCTVTSLLPVG | |
| | PVLWFRGVGPGRELIYVSGRGHFPRVTTVSDLTK | |
| | RENRDFSIRISSITPADVGTYYCVKFRKGSPEDVE | |
| | FKSGPGTEMALGAKPS | |
| | (SEQ ID NO: 64) | |
| h1831-S12 | QVQLVQSGAEVKKPGASVKVSCKASGYTFTSY | |
| | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPRCPA | |
| | PEFLGGPSVFLFPPKPKDTLMISRTPEVTCVVVD | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPPV | |
| | LDSDGSFFLYSRLTVDKSRWQEGNVFSCSVMHE | |
| | ALHNHYTQKSLSLSLGGGGGSGGGGSGGGGGG | |
| | EEELQMIQPEKLLLVTVGETATLHCTVTSLLPVG | |
| | PVLWFRGVGPGRELIYVSGRGHFPRVTTVSDLTK | |
| | RENKDFSIRISSITPADVGTYYCVKFRKGSPEDVE | |
| | FKSGPGTEMALGAKPS | |
| | (SEQ ID NO: 65) | |
| h1831-S19 | QVQLVQSGAEVKKPGASVKVSCKASGYTFTSY | |
| | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPRCPA | |
| | PEFLGGPSVFLFPPKPKDTLMISRTPEVTCVVVD | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPPV | |
| | LDSDGSFFLYSRLTVDKSRWQEGNVFSCSVMHE | |
| | ALHNHYTQKSLSLSLGGGGGSGGGGSGGGGGG | |
| | EEELQMIQPEKLLLVTVGETATLHCTVTSLLPVG | |
| | PVLWFRGVGPGRELIYVSGRGHFPRVTTVSDLTK | |
| | RNNRDFSIRISSITPADVGTYYCVKFRKGSPEDVE | |
| | FKSGPGTEMALGAKPS | |
| | (SEQ ID NO: 66) | |
| h1831-S85 | QVQLVQSGAEVKKPGASVKVSCKASGYTFTSY | |
| | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPRCPA | |
| | peflggpsvflfppkpkdtlmisrtpevtcvvvd | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPPV | |
| | LDSDGSFFLYSRLTVDKSRWQEGNVFSCSVMHE | |
| | ALHNHYTQKSLSLSLGGGGGSGGGGSGGGGGG | |
| | EEELQMIQPEKLLLVTVGETATLHCTVTSLLPVG | |
| | PVLWFRGVGPGRELIYVSGRGHFPRVTTVSDLTK | |
| | RNNKDFSIRISSITPADVGTYYCVKFRKGSPEDVE | |
| | FKSGPGTEMALGAKPS | |
| | (SEQ ID NO: 67) | |
| h1831-S37 | QVQLVQSGAEVKKPGASVKVSCKASGYTFTSY | |
| | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPRCPA | |
| | peflggpsvflfppkpkdtlmisrtpevtcvvvd | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPPV | |
| | LDSDGSFFLYSRLTVDKSRWQEGNVFSCSVMHE | |
| | ALHNHYTQKSLSLSLGGGGGSGGGGSGGGGGG | |
| | EEELQMIQPEKLLLVTVGETATLHCTVTSLLPVG | |
| | PVLWFRGVGPGRELIYMSGRGHFPRVTTVSDLT | |
| | KRNNKDFSIRISSITPADVGTYYCVKFRKGSPEDV | |
| | EFKSGPGTEMALGAKPS | |
| | (SEQ ID NO: 68) | |
| h1831-19- | QVQLVQSGAEVKKPGASVKVSCKASGYTFTSY | |
| S37 | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGTK | |
| | TYTCNVDHKPSNTKVDKRVESKYGPPCPPCPAPE | |
| | FLGGPSVFLFPPKPKDTLMISRTPEVTCVVVDVS | |
| | QEDPEVQFNWYVDGVEVHNAKTKPREEQFNST | |
| | YRVVSVLTVLHQDWLNGKEYKCKVSNKGLPSSI | |
| | EKTISKAKGQPREPQVYTLPPSQEEMTKNQVSLT | |
| | CLVKGFYPSDIAVEWESNGQPENNYKTTPPVLDS | |
| | DGSFFLYSRLTVDKSRWQEGNVFSCSVMHEALH | |
| | NHYTQKSLSLSLGGGGGSGGGGSGGGGSGGGG | |
| | EEELQMIQPEKLLLVTVGETATLHCTVTSLLPVG | |
| | PVLWFRGVGPGRELIYMSGRGHFPRVTTVSDLT | |
| | KRNNKDFSIRISSITPADVGTYYCVKFRKGSPEDV | |
| | EFKSGPGTEMALGAKPS | |
| | (SEQ ID NO: 109) | |
| h1831-19- | QVQLVQSGAEVKKPGASVKVSCKASGYTFTSY | |
| S79 | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGTK | |
| | TYTCNVDHKPSNTKVDKRVESKYGPPCPPCPAPE | |
| | FLGGPSVFLFPPKPKDTLMISRTPEVTCVVVDVS | |
| | QEDPEVQFNWYVDGVEVHNAKTKPREEQFNST | |
| | YRVVSVLTVLHQDWLNGKEYKCKVSNKGLPSSI | |
| | EKTISKAKGQPREPQVYTLPPSQEEMTKNQVSLT | |
| | CLVKGFYPSDIAVEWESNGQPENNYKTTPPVLDS | |
| | DGSFFLYSRLTVDKSRWQEGNVFSCSVMHEALH | |
| | NHYTQKSLSLSLGGGGGSGGGGSGGGGSGGGG | |
| | EEELQMIQPEKLLLVTVGETATLHCTVTSLWPVG | |
| | PVLWFRGVGPGRELIYRTGTGRFPRVTTVSDLTK | |
| | RNNMDFSIRISSITPADVGTYYCVKFRKGSPEDV | |
| | EFKSGPGTEVALGAKPS | |
| | (SEQ ID NO: 69) | |
| h1831-S38 | QVQLVQSGAEVKKPGASVKVSCKASGYTFTSY | |
| | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPRCPA | |
| | peflggpsvflfppkpkdtlmisrtpevtcvvvd | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPPV | |
| | LDSDGSFFLYSRLTVDKSRWQEGNVFSCSVMHE | |
| | ALHNHYTQKSLSLSLGGGGGSGGGGSGGGGGG | |
| | EEELQMIQPEKLLLVTVGETATLHCTVTSLLPVG | |
| | PVLWFRGVGPGRELIYFSGRGHFPRVTTVSDLTK | |
| | RNNMDFSIRISSITPADVGTYYCVKFRKGSPEDV | |
| | EFKSGPGTEMALGAKPS | |
| | (SEQ ID NO: 70) | |
| h1831-S22 | QVQLVQSGAEVKKPGASVKVSCKASGYTFTSY | |
| | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPRCPA | |
| | peflggpsvflfppkpkdtlmisrtpevtcvvvd | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPPV | |
| | LDSDGSFFLYSRLTVDKSRWQEGNVFSCSVMHE | |
| | ALHNHYTQKSLSLSLGGGGGSGGGGSGGGGGG | |
| | EEELQMIQPEKLLLVTVGETATLHCTVTSLLPVG | |
| | PVLWFRGVGPGRELIYISGRGHFPRVTTVSDLTK | |
| | RNNMDFSIRISSITPADVGTYYCVKFRKGSPEDV | |
| | EFKSGPGTEMALGAKPS | |
| | (SEQ ID NO: 71) | |
| h1831-S29 | QVQLVQSGAEVKKPGASVKVSCKASGYTFTSY | |
| | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPRCPA | |
| | peflggpsvflfppkpkdtlmisrtpevtcvvvd | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPPV | |
| | LDSDGSFFLYSRLTVDKSRWQEGNVFSCSVMHE | |
| | ALHNHYTQKSLSLSLGGGGGSGGGGSGGGGGG | |
| | EEELQMIQPEKLLLVTVGETATLHCTVTSLLPVG | |
| | PVLWFRGVGPGRELIYLSGRGHFPRVTTVSDLTK | |
| | RNNMDFSIRISSITPADVGTYYCVKFRKGSPEDV | |
| | EFKSGPGTEMALGAKPS | |
| | (SEQ ID NO: 72) | |
| M831-S34 | QVQLVQSGAEVKKPGASVKVSCKASGYTFTSY | |
| | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPRCPA | |
| | PEFLGGPSVFLFPPKPKDTLMISRTPEVTCVVVD | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPPV | |
| | LDSDGSFFLYSRLTVDKSRWQEGNVFSCSVMHE | |
| | ALHNHYTQKSLSLSLGGGGGSGGGGSGGGGGG | |
| | EEELQMIQPEKLLLVTVGETATLHCTVTSLLPVG | |
| | PVLWFRGVGPGRELIYRSGRGHFPRVTTVSDLTK | |
| | RNNMDFSIRISSITPADVGTYYCVKFRKGSPEDV | |
| | EFKSGPGTEMALGAKPS | |
| | (SEQ ID NO: 73) | |
| h1831-S41 | QVQLVQSGAEVKKPGASVKVSCKASGYTFTSY | |
| | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPPCPA | |
| | PEFLGGPSVFLFPPKPKDTLMISRTPEVTCVVVD | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPPV | |
| | LDSDGSFFLYSRLTVDKSRWQEGNVFSCSVMHE | |
| | ALHNHYTQKSLSLSLGGGGGSGGGGSGGGGGG | |
| | EEELQMIQPEKLLLVTVGETATLHCTVTSLLPVG | |
| | PVLWFRGVGPGRELIYVSGRGHFPRVTTVSDLTK | |
| | RNNMDFSIRISSITPADVGTYYCVKFRKGSPEDV | |
| | EFKSGPGTEMALGAKPS | |
| | (SEQ ID NO: 74) | |
| h1831-S42 | QVQLVQSGAEVKKPGASVKVSCKASGYTFTSY | |
| | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPRCPA | |
| | peflggpsvflfppkpkdtlmisrtpevtcvvvd | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPPV | |
| | LDSDGSFFLYSRLTVDKSRWQEGNVFSCSVMHE | |
| | ALHNHYTQKSLSLSLGGGGGSGGGGSGGGGGG | |
| | EEELQMIQPEKLLLVTVGETATLHCTVTSLLPVG | |
| | PVLWFRGVGPGRELIYMSGRGHFPRVTTVSDLT | |
| | KRNNIDFSIRISSITPADVGTYYCVKFRKGSPEDV | |
| | EFKSGPGTEMALGAKPS | |
| | (SEQ ID NO: 75) | |
| h1831-S43 | QVQLVQSGAEVKKPGASVKVSCKASGYTFTSY | |
| | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPRCPA | |
| | peflggpsvflfppkpkdtlmisrtpevtcvvvd | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPPV | |
| | LDSDGSFFLYSRLTVDKSRWQEGNVFSCSVMHE | |
| | ALHNHYTQKSLSLSLGGGGGSGGGGSGGGGGG | |
| | EEELQMIQPEKLLLVTVGETATLHCTVTSLLPVG | |
| | PVLWFRGVGPGRELIYMSGRGHFPRVTTVSDLT | |
| | KRNNRDFSIRISSITPADVGTYYCVKFRKGSPEDV | |
| | EFKSGPGTEMALGAKPS | |
| | (SEQ ID NO: 76) | |
| h1831-S44 | QVQLVQSGAEVKKPGASVKVSCKASGYTFTSY | |
| | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPPCPA | |
| | peflggpsvflfppkpkdtlmisrtpevtcvvvd | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPPV | |
| | LDSDGSFFLYSRLTVDKSRWQEGNVFSCSVMHE | |
| | ALHNHYTQKSLSLSLGGGGGSGGGGSGGGGGG | |
| | EEELQMIQPEKLLLVTVGETATLHCTVTSLLPVG | |
| | PVLWFRGVGPGRELIYMSGRGHFPRVTTVSDLT | |
| | KRNNVDFSIRISSITPADVGTYYCVKFRKGSPEDV | |
| | EFKSGPGTEMALGAKPS | |
| | (SEQ ID NO: 77) | |
| M831-S45 | QVQLVQSGAEVKKPGASVKVSCKASGYTFTSY | |
| | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPRCPA | |
| | peflggpsvflfppkpkdtlmisrtpevtcvvvd | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPPV | |
| | LDSDGSFFLYSRLTVDKSRWQEGNVFSCSVMHE | |
| | ALHNHYTQKSLSLSLGGGGGSGGGGSGGGGGG | |
| | EEELQMIQPEKLLLVTVGETATLHCTVTSLLPVG | |
| | PVLWFRGVGPGRELIYMSGRGHFPRVTTVSDLT | |
| | KRDNMDFSIRISSITPADVGTYYCVKFRKGSPED | |
| | VEFKSGPGTEMALGAKPS | |
| | (SEQ ID NO: 78) | |
| h1831-S46 | QVQLVQSGAEVKKPGASVKVSCKASGYTFTSY | |
| | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPRCPA | |
| | PEFLGGPSVFLFPPKPKDTLMISRTPEVTCVVVD | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPPV | |
| | LDSDGSFFLYSRLTVDKSRWQEGNVFSCSVMHE | |
| | ALHNHYTQKSLSLSLGGGGGSGGGGSGGGGGG | |
| | EEELQMIQPEKLLLVTVGETATLHCTVTSLLPVG | |
| | PVLWFRGVGPGRELIYMSGRGHFPRVTTVSDLT | |
| | KRENKDFSIRISSITPADVGTYYCVKFRKGSPEDV | |
| | EFKSGPGTEMALGAKPS | |
| | (SEQ ID NO: 79) | |
| h1831-S47 | QVQLVQSGAEVKKPGASVKVSCKASGYTFTSY | |
| | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPRCPA | |
| | PEFLGGPSVFLFPPKPKDTLMISRTPEVTCVVVD | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPPV | |
| | LDSDGSFFLYSRLTVDKSRWQEGNVFSCSVMHE | |
| | ALHNHYTQKSLSLSLGGGGGSGGGGSGGGGGG | |
| | EEELQMIQPEKLLLVTVGETATLHCTVTSLLPVG | |
| | PVLWFRGVGPGRELIYMSGRGHFPRVTTVSDLT | |
| | KRENKDFSIRISSITPADVGTYYCVKFRKGSPEDV | |
| | EFKSGPGTEVALGAKPS | |
| | (SEQ ID NO: 80) | |
| h1831-S48 | QVQLVQSGAEVKKPGASVKVSCKASGYTFTSY | |
| | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPRCPA | |
| | PEFLGGPSVFLFPPKPKDTLMISRTPEVTCVVVD | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPPV | |
| | LDSDGSFFLYSRLTVDKSRWQEGNVFSCSVMHE | |
| | ALHNHYTQKSLSLSLGGGGGSGGGGSGGGGGG | |
| | EEELQMIQPEKLLLVTVGETATLHCTVTSLLPVG | |
| | PVLWFRGVGPGRELIYRSGRGHFPRVTTVSDLTK | |
| | RNNKDFSIRISSITPADVGTYYCVKFRKGSPEDVE | |
| | FKSGPGTEMALGAKPS | |
| | (SEQ ID NO: 81) | |
| h1831-S49 | QVQLVQSGAEVKKPGASVKVSCKASGYTFTSY | |
| | WMHWVRQAPGQGLEWMGRITPSSGFAMYNEK | |
| | FKNRVTMTRDTSTSTVYMELSSLRSEDTAVYYC | |
| | ARGGSSYDYFDYWGQGTTVTVSSASTKGPSVFP | |
| | LAPCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPPCPA | |
| | PEFLGGPSVFLFPPKPKDTLMISRTPEVTCVVVD | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPPV | |
| | LDSDGSFFLYSRLTVDKSRWQEGNVFSCSVMHE | |
| | ALHNHYTQKSLSLSLGGGGGSGGGGSGGGGGG | |
| | EEELQMIQPEKLLLVTVGETATLHCTVTSLLPVG | |
| | PVLWFRGVGPGRELIYRSGRGHFPRVTTVSDLTK | |
| | RENKDFSIRISSITPADVGTYYCVKFRKGSPEDVE | |
| | FKSGPGTEMALGAKPS | |
| | (SEQ ID NO: 82) | |

The following proteins were also prepared and purified by conventional methods as positive or negative controls.

Anti-CD47 Antibody hu5F9 (the Sequence was from U.S. Ser. No. 09/017,675B)

Hu5F9 heavy chain
(SEQ ID NO: 83)
QVQLVQSGAEVKKPGASVKVSCKASGYTFTNYNMH
WVRQAPGQRLEWMGTIYPGNDDTSYNQKFKDRVTI
TADTSASTAYMELSSLRSEDTAVYYCARGGYRAMD
YWGQGTLVTVSSASTKGPSVFPLAPCSRSTSESTA
ALGCLVKDYFPEPVTVSWNSGALTSGVHTFPAVLQ
SSGLYSLSSVVTVPSSSLGTKTYTCNVDHKPSNTK
VDKRVESKYGPPCPPCPAPEFLGGPSVFLFPPKPK
DTLMISRTPEVTCVVVDVSQEDPEVQFNWYVDGVE
VHNAKTKPREEQFNSTYRVVSVLTVLHQDWLNGKE
YKCKVSNKGLPSSIEKTISKAKGQPREPQVYTLPP
SQEEMTKNQVSLTCLVKGFYPSDIAVEWESNGQPE
NNYKTTPPVLDSDGSFFLYSRLTVDKSRWQEGNVF
SCSVMHEALHNHYTQKSLSLSLGK;
Hu5F9 light chain
(SEQ ID NO: 84)
DIVMTQSPLSLPVTPGEPASISCRSSQSIVYSNGN
TYLGWYLQKPGQSPQLLIYKVSNRFSGVPDRFSGS
GSGTDFTLKISRVEAEDVGVYYCFQGSHVPYTFGQ
GTKLEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC;
SIRPα-CV (synthesized referring to
Engineered SIRPα Variants as
Immunotherapeutic Adjuvants to
Anticancer Antibodies, Science.
2013 Jul 5; 341(6141):88-91, SEQ ID NO: 85)
EEELQIIQPDKSVLVAAGETATLRCTITSLFPVGP
IQWFRGAGPGRVLIYNQRQGPFPRVTTVSDTTKRN
NMDFSIRIGNITPADAGTYYCIKFRKGSPDDVEFK
SGAGTELSVRAKPSEPKSSDKTHTCPPCPAPELLG
GPSVFLFPPKPKDTLMISRTPEVTCVVVDVSHEDP
EVKFNWYVDGVEVHNAKTKPREEQYNSTYRVVSVL
TVLHQDWLNGKEYKCKVSNKALPAPIEKTISKAKG
QPREPQVYTLPPSRDELTKNQVSLTCLVKGFYPSD
IAVEWESNGQPENNYKTTPPVLDSDGSFFLYSKLT
VDKSRWQQGNVFSCSVMHEALHNHYTQKSLSLSPG
K;
TTI-621:
(sequence from WO2014094122A1, SEQ ID NO: 133)
EEELQVIQPDKSVSVAAGES
AILHCTVTSLIPVGPIQWFRGAGPARELIYNQKEG
HFPRVTTVSESTKRENMDFSISISNITPADAGTYY
CVKFRKGSPDTEFKSGAGTELSVRAKPSDKTHTCP
PCPAPELLGGPSVFLFPPKPKDTLMISRTPEVTCV
VVDVSHEDPEVKFNWYVDGVEVHNAKTKPREEQYN
STYRVVSVLTVLHQDWLNGKEYKCKVSNKALPAPI
EKTISKAKGQPREPQVYTLPPSRDELTKNQVSLTC
LVKGFYPSDIAVEWESNGQPENNYKTTPPVLDSDG
SFFLYSKLTVDKSRWQQGNVFSCSVMHEALHNHYT
QKSLSLSPGK;
S58-Fc
(SEQ ID NO: 86)
EEELQMIQPEKLLLVTVGETATLHCTVTSLLPVGP
VLWFRGVGPGRELIYMSGRGHFPRVTTVSDLTKRN
NMDFSIRISSITPADVGTYYCVKFRKGSPEDVEFK
SGPGTEMALGAKPSESKYGPPCPPCPAPEFLGGPS
VFLFPPKPKDTLMISRTPEVTCVVVDVSQEDPEVQ
FNWYVDGVEVHNAKTKPREEQFNSTYRVVSVLTVL
HQDWLNGKEYKCKVSNKGLPSSIEKTISKAKGQPR
EPQVYTLPPSQEEMTKNQVSLTCLVKGFYPSDIAV
EWESNGQPENNYKTTPPVLDSDGSFFLYSRLTVDK
SRWQEGNVFSCSVMHEALHNHYTQKSLSLSLGK;

### Anti-CD47 Antibody Hu167-IgG4 AA (Prepared According to the Method Disclosed in Patent Application WO2018095428A1)

Hu167-IgG4 AA heavy chain
(SEQ ID NO: 87)
QVQLVQSGAEVKKPGASVKVSCKASGYTFTSYWMH
WVRQAPGQGLEWMGNIDPSDSETHYNQKFKDRVTM
TRDTSISTAYMELSRLRSDDTAVYYCARWGYLGRS
AMDYWGQGTTVTVSSASTKGPSVFPLAPCSRSTSE
STAALGCLVKDYFPEPVTVSWNSGALTSGVHTFPA
VLQSSGLYSLSSVVTVPSSSLGTKTYTCNVDHKPS
NTKVDKRVESKYGPPCPPCPAPEAAGGPSVFLFPP
KPKDTLMISRTPEVTCVVVDVSQEDPEVQFNWYVD
GVEVHNAKTKPREEQFNSTYRVVSVLTVLHQDWLN
GKEYKCKVSNKGLPSSIEKTISKAKGQPREPQVYT
LPPSQEEMTKNQVSLTCLVKGFYPSDIAVEWESNG
QPENNYKTTPPVLDSDGSFFLYSRLTVDKSRWQEG
NVFSCSVMHEALHNHYTQKSLSLSLGK;
Hu167-IgG4 AA light chain
(SEQ ID NO: 88)
DVQITQSPSSLSASVGDRVTITCRTSKSISKFLAW
YQQKPGKAPKLLIYSGSTLQSGVPSRFSGSGSGTD
FTLTISSLQPEDFATYYCQQHNEYPWTFGGGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC;
S37-Fc
(SEQ ID NO: 110)
EEELQMIQPEKLLLVTVGETATLHCTVTSLLPVGP
VLWFRGVGPGRELIYMSGRGHFPRVTTVSDLTKRN
NKDFSIRISSITPADVGTYYCVKFRKGSPEDVEFK
SGPGTEMALGAKPSESKYGPPCPPCPAPEFLGGPS
VFLFPPKPKDTLMISRTPEVTCVVVDVSQEDPEVQ
FNWYVDGVEVHNAKTKPREEQFNSTYRVVSVLTVL
HQDWLNGKEYKCKVSNKGLPSSIEKTISKAKGQPR
EPQVYTLPPSQEEMTKNQVSLTCLVKGFYPSDIAV
EWESNGQPENNYKTTPPVLDSDGSFFLYSRLTVDK
SRWQEGNVFSCSVMHEALHNHYTQKSLSLSLGK.

Antibody h1831K

The h1831 antibody was subjected to CDR mutation modification, and 36 mutants were obtained, and finally the N53K (position determined according to the Kabat numbering criteria) mutant h1831K was selected. The h1831 light chain LCDR2 was mutated from AASNLES to AASKLES, to obtain a new antibody h1831K.

h1831 light chain: (SEQ ID NO: 111)
DIVLTQSPASLAVSPGQRATITCRASESVSIHGTHLMHWY
QQKPGQPPKLLIYAASKLESGVPARFSGSGSGTDFTLTIN
PVEAEDTANYYCQQSFEDPLTFGQGTKLEIKRTVAAPSVF
IFPPSDEQLKSGTASVVCLLNNFYPREAKVQWKVDNALQS
GNSQESVTEQDSKDSTYSLSSTLTLSKADYEKHKVYACEV
THQGLSSPVTKSFNRGEC;
wherein LCDR1 was
RASESVSIHGTHLMH (SEQ ID NO: 106),
LCDR2 was AASKLES (SEQ ID NO: 112),
LCDR3 was QQSFEDPLT (SEQ ID NO: 108).
h1831K light chain variable region
(SEQ ID NO: 113)
DIVLTQSPASLAVSPGQRATITCRASESVSIHGTHLMHWY
QQKPGQPPKLLIYAASKLESGVPARFSGSGSGTDFTLTIN
PVEAEDTANYYCQQSFEDPLTFGQGTKLEIK;
h1831K heavy chain
(same as h1831 heavy chain sequence,
SEQ ID NO: 16)
QVQLVQSGAEVKKPGASVKVSCKASGYTFTSYWMHWVRQA
PGQGLEWMGRITPSSGFAMYNEKFKNRVTMTRDTSTSTVY
MELSSLRSEDTAVYYCARGGSSYDYFDYWGQGTTVTVSSA
STKGPSVFPLAPCSRSTSESTAALGCLVKDYFPEPVTVSW
NSGALTSGVHTFPAVLQSSGLYSLSSVVTVPSSSLGTKTY
TCNVDHKPSNTKVDKRVESKYGPPCPPCPAPEFLGGPSVF
LFPPKPKDTLMISRTPEVTCVVVDVSQEDPEVQFNWYVDG
VEVHNAKTKPREEQFNSTYRVVSVLTVLHQDWLNGKEYKC
KVSNKGLPSSIEKTISKAKGQPREPQVYTLPPSQEEMTKN
QVSLTCLVKGFYPSDIAVEWESNGQPENNYKTTPPVLDSD
GSFFLYSRLTVDKSRWQEGNVFSCSVMHEALHNHYTQKSL
SLSLG;
>h1831K-19-S37 heavy chain (same as h1831-19-
S37 heavy chain sequence, SEQ ID NO: 109)
QVQLVQSGAEVKKPGASVKVSCKASGYTFTSYWMHWVRQA
PGQGLEWMGRITPSSGFAMYNEKFKNRVTMTRDTSTSTVY
MELSSLRSEDTAVYYCARGGSSYDYFDYWGQGTTVTVSSA
STKGPSVFPLAPCSRSTSESTAALGCLVKDYFPEPVTVSW
NSGALTSGVHTFPAVLQSSGLYSLSSVVTVPSSSLGTKTY
TCNVDHKPSNTKVDKRVESKYGPPCPPCPAPEFLGGPSVF
LFPPKPKDTLMISRTPEVTCVVVDVSQEDPEVQFNWYVDG
VEVHNAKTKPREEQFNSTYRVVSVLTVLHQDWLNGKEYKC
KVSNKGLPSSIEKTISKAKGQPREPQVYTLPPSQEEMTKN
QVSLTCLVKGFYPSDIAVEWESNGQPENNYKTTPPVLDSD
GSFFLYSRLTVDKSRWQEGNVFSCSVMHEALHNHYTQKSL
SLSLGGGGGSGGGGSGGGGSGGGGEEELQMIQPEKLLLVT
VGETATLHCTVTSLLPVGPVLWFRGVGPGRELIYMSGRGH
FPRVTTVSDLTKRNNKDFSIRISSITPADVGTYYCVKFRK
GSPEDVEFKSGPGTEMALGAKPS;
> h1831K-19-S37 light chain (same as
h1831K light chain sequence, SEQ ID NO: 111)
DIVLTQSPASLAVSPGQRATITCRASESVSIHGTHLMHWY
QQKPGQPPKLLIYAASKLESGVPARFSGSGSGTDFTLTIN
PVEAEDTANYYCQQSFEDPLTFGQGTKLEIKRTVAAPSVF
IFPPSDEQLKSGTASVVCLLNNFYPREAKVQWKVDNALQS
GNSQESVTEQDSKDSTYSLSSTLTLSKADYEKHKVYACEV
THQGLSSPVTKSFNRGEC;
S79-Fc (SEQ ID NO: 114)
EEELQMIQPEKLLLVTVGETATLHCTVTSLWPVGPVLWFR
GVGPGRELIYRTGTGRFPRVTTVSDLTKRNNMDFSIRISS
ITPADVGTYYCVKFRKGSPEDVEFKSGPGTEVALGAKPSE
SKYGPPCPPCPAPEFLGGPSVFLFPPKPKDTLMISRTPEV
TCVVVDVSQEDPEVQFNWYVDGVEVHNAKTKPREEQFNST
YRVVSVLTVLHQDWLNGKEYKCKVSNKGLPSSIEKTISKA
KGQPREPQVYTLPPSQEEMTKNQVSLTCLVKGFYPSDIAV
EWESNGQPENNYKTTPPVLDSDGSFFLYSRLTVDKSRWQE
GNVFSCSVMHEALHNHYTQKSLSLSLGK;
S15-Fc (SEQ ID NO: 115)
EEELQMIQPEKLLLVTVGETATLHCTVTSLLPVGPVLWFR
GVGPGRELIYVSGRGHFPRVTTVSDLTKRENRDFSIRISS
ITPADVGTYYCVKFRKGSPEDVEFKSGPGTEMALGAKPSE
SKYGPPCPPCPAPEFLGGPSVFLFPPKPKDTLMISRTPEV
TCVVVDVSQEDPEVQFNWYVDGVEVHNAKTKPREEQFNST
YRVVSVLTVLHQDWLNGKEYKCKVSNKGLPSSIEKTISKA
KGQPREPQVYTLPPSQEEMTKNQVSLTCLVKGFYPSDIAV
EWESNGQPENNYKTTPPVLDSDGSFFLYSRLTVDKSRWQE
GNVFSCSVMHEALHNHYTQKSLSLSLGK;
S12-Fc (SEQ ID NO: 116)
EEELQMIQPEKLLLVTVGETATLHCTVTSLLPVGPVLWFR
GVGPGRELIYVSGRGHFPRVTTVSDLTKRENKDFSIRISS
ITPADVGTYYCVKFRKGSPEDVEFKSGPGTEMALGAKPSE
SKYGPPCPPCPAPEFLGGPSVFLFPPKPKDTLMISRTPEV
TCVVVDVSQEDPEVQFNWYVDGVEVHNAKTKPREEQFNST
YRVVSVLTVLHQDWLNGKEYKCKVSNKGLPSSIEKTISKA
KGQPREPQVYTLPPSQEEMTKNQVSLTCLVKGFYPSDIAV
EWESNGQPENNYKTTPPVLDSDGSFFLYSRLTVDKSRWQE
GNVFSCSVMHEALHNHYTQKSLSLSLGK;
S19-Fc (SEQ ID NO: 117)
EEELQMIQPEKLLLVTVGETATLHCTVTSLLPVGPVLWFR
GVGPGRELIYVSGRGHFPRVTTVSDLTKRNNRDFSIRISS
ITPADVGTYYCVKFRKGSPEDVEFKSGPGTEMALGAKPSE
SKYGPPCPPCPAPEFLGGPSVFLFPPKPKDTLMISRTPEV
TCVVVDVSQEDPEVQFNWYVDGVEVHNAKTKPREEQFNST
YRVVSVLTVLHQDWLNGKEYKCKVSNKGLPSSIEKTISKA
KGQPREPQVYTLPPSQEEMTKNQVSLTCLVKGFYPSDIAV
EWESNGQPENNYKTTPPVLDSDGSFFLYSRLTVDKSRWQE
GNVFSCSVMHEALHNHYTQKSLSLSLGK;
S85-Fc (SEQ ID NO: 118)
EEELQMIQPEKLLLVTVGETATLHCTVTSLLPVGPVLWFR
GVGPGRELIYVSGRGHFPRVTTVSDLTKRNNKDFSIRISS
ITPADVGTYYCVKFRKGSPEDVEFKSGPGTEMALGAKPSE
SKYGPPCPPCPAPEFLGGPSVFLFPPKPKDTLMISRTPEV
TCVVVDVSQEDPEVQFNWYVDGVEVHNAKTKPREEQFNST
YRVVSVLTVLHQDWLNGKEYKCKVSNKGLPSSIEKTISKA
KGQPREPQVYTLPPSQEEMTKNQVSLTCLVKGFYPSDIAV
EWESNGQPENNYKTTPPVLDSDGSFFLYSRLTVDKSRWQE
GNVFSCSVMHEALHNHYTQKSLSLSLGK;
S38-Fc (SEQ ID NO: 119)
EEELQMIQPEKLLLVTVGETATLHCTVTSLLPVGPVLWFR
GVGPGRELIYFSGRGHFPRVTTVSDLTKRNNMDFSIRISS
ITPADVGTYYCVKFRKGSPEDVEFKSGPGTEMALGAKPSE
SKYGPPCPPCPAPEFLGGPSVFLFPPKPKDTLMISRTPEV
TCVVVDVSQEDPEVQFNWYVDGVEVHNAKTKPREEQFNST
YRVVSVLTVLHQDWLNGKEYKCKVSNKGLPSSIEKTISKA
KGQPREPQVYTLPPSQEEMTKNQVSLTCLVKGFYPSDIAV
EWESNGQPENNYKTTPPVLDSDGSFFLYSRLTVDKSRWQE
GNVFSCSVMHEALHNHYTQKSLSLSLGK;
S22-Fc (SEQ ID NO: 120)
EEELQMIQPEKLLLVTVGETATLHCTVTSLLPVGPVLWFR
GVGPGRELIYISGRGHFPRVTTVSDLTKRNNMDFSIRISS
ITPADVGTYYCVKFRKGSPEDVEFKSGPGTEMALGAKPSE
SKYGPPCPPCPAPEFLGGPSVFLFPPKPKDTLMISRTPEV
TCVVVDVSQEDPEVQFNWYVDGVEVHNAKTKPREEQFNST
YRVVSVLTVLHQDWLNGKEYKCKVSNKGLPSSIEKTISKA
KGQPREPQVYTLPPSQEEMTKNQVSLTCLVKGFYPSDIAV
EWESNGQPENNYKTTPPVLDSDGSFFLYSRLTVDKSRWQE
GNVFSCSVMHEALHNHYTQKSLSLSLGK;
S29-Fc (SEQ ID NO: 121)
EEELQMIQPEKLLLVTVGETATLHCTVTSLLPVGPVLWFR
GVGPGRELIYLSGRGHFPRVTTVSDLTKRNNMDFSIRISS
ITPADVGTYYCVKFRKGSPEDVEFKSGPGTEMALGAKPSE
SKYGPPCPPCPAPEFLGGPSVFLFPPKPKDTLMISRTPEV
TCVVVDVSQEDPEVQFNWYVDGVEVHNAKTKPREEQFNST
YRVVSVLTVLHQDWLNGKEYKCKVSNKGLPSSIEKTISKA
KGQPREPQVYTLPPSQEEMTKNQVSLTCLVKGFYPSDIAV
EWESNGQPENNYKTTPPVLDSDGSFFLYSRLTVDKSRWQE
GNVFSCSVMHEALHNHYTQKSLSLSLGK;
S34-Fc (SEQ ID NO: 122)
EEELQMIQPEKLLLVTVGETATLHCTVTSLLPVGPVLWFR
GVGPGRELIYRSGRGHFPRVTTVSDLTKRNNMDFSIRISS
ITPADVGTYYCVKFRKGSPEDVEFKSGPGTEMALGAKPSE
SKYGPPCPPCPAPEFLGGPSVFLFPPKPKDTLMISRTPEV
TCVVVDVSQEDPEVQFNWYVDGVEVHNAKTKPREEQFNST
YRVVSVLTVLHQDWLNGKEYKCKVSNKGLPSSIEKTISKA
KGQPREPQVYTLPPSQEEMTKNQVSLTCLVKGFYPSDIAV
EWESNGQPENNYKTTPPVLDSDGSFFLYSRLTVDKSRWQE
GNVFSCSVMHEALHNHYTQKSLSLSLGK;
S41-Fc (SEQ ID NO: 123)
EEELQMIQPEKLLLVTVGETATLHCTVTSLLPVGPVLWFR
GVGPGRELIYVSGRGHFPRVTTVSDLTKRNNMDFSIRISS
ITPADVGTYYCVKFRKGSPEDVEFKSGPGTEMALGAKPSE
SKYGPPCPPCPAPEFLGGPSVFLFPPKPKDTLMISRTPEV
TCVVVDVSQEDPEVQFNWYVDGVEVHNAKTKPREEQFNST
YRVVSVLTVLHQDWLNGKEYKCKVSNKGLPSSIEKTISKA
KGQPREPQVYTLPPSQEEMTKNQVSLTCLVKGFYPSDIAV
EWESNGQPENNYKTTPPVLDSDGSFFLYSRLTVDKSRWQE
GNVFSCSVMHEALHNHYTQKSLSLSLGK;
S42-Fc (SEQ ID NO: 124)
EEELQMIQPEKLLLVTVGETATLHCTVTSLLPVGPVLWFR
GVGPGRELIYMSGRGHFPRVTTVSDLTKRNNIDFSIRISS
ITPADVGTYYCVKFRKGSPEDVEFKSGPGTEMALGAKPSE
SKYGPPCPPCPAPEFLGGPSVFLFPPKPKDTLMISRTPEV
TCVVVDVSQEDPEVQFNWYVDGVEVHNAKTKPREEQFNST
YRVVSVLTVLHQDWLNGKEYKCKVSNKGLPSSIEKTISKA
KGQPREPQVYTLPPSQEEMTKNQVSLTCLVKGFYPSDIAV
EWESNGQPENNYKTTPPVLDSDGSFFLYSRLTVDKSRWQE
GNVFSCSVMHEALHNHYTQKSLSLSLGK;
S43-Fc (SEQ ID NO: 125)
EEELQMIQPEKLLLVTVGETATLHCTVTSLLPVGPVLWFR
GVGPGRELIYMSGRGHFPRVTTVSDLTKRNNRDFSIRISS
ITPADVGTYYCVKFRKGSPEDVEFKSGPGTEMALGAKPSE
SKYGPPCPPCPAPEFLGGPSVFLFPPKPKDTLMISRTPEV
TCVVVDVSQEDPEVQFNWYVDGVEVHNAKTKPREEQFNST
YRVVSVLTVLHQDWLNGKEYKCKVSNKGLPSSIEKTISKA
KGQPREPQVYTLPPSQEEMTKNQVSLTCLVKGFYPSDIAV
EWESNGQPENNYKTTPPVLDSDGSFFLYSRLTVDKSRWQE
GNVFSCSVMHEALHNHYTQKSLSLSLGK;
S44-Fc (SEQ ID NO: 126)
EEELQMIQPEKLLLVTVGETATLHCTVTSLLPVGPVLWFR
GVGPGRELIYMSGRGHFPRVTTVSDLTKRNNVDFSIRISS
ITPADVGTYYCVKFRKGSPEDVEFKSGPGTEMALGAKPSE
SKYGPPCPPCPAPEFLGGPSVFLFPPKPKDTLMISRTPEV
TCVVVDVSQEDPEVQFNWYVDGVEVHNAKTKPREEQFNST
YRVVSVLTVLHQDWLNGKEYKCKVSNKGLPSSIEKTISKA
KGQPREPQVYTLPPSQEEMTKNQVSLTCLVKGFYPSDIAV
EWESNGQPENNYKTTPPVLDSDGSFFLYSRLTVDKSRWQE
GNVFSCSVMHEALHNHYTQKSLSLSLGK;
S45-Fc (SEQ ID NO: 127)
EEELQMIQPEKLLLVTVGETATLHCTVTSLLPVGPVLWFR
GVGPGRELIYMSGRGHFPRVTTVSDLTKRDNMDFSIRISS
ITPADVGTYYCVKFRKGSPEDVEFKSGPGTEMALGAKPSE
SKYGPPCPPCPAPEFLGGPSVFLFPPKPKDTLMISRTPEV
TCVVVDVSQEDPEVQFNWYVDGVEVHNAKTKPREEQFNST
YRVVSVLTVLHQDWLNGKEYKCKVSNKGLPSSIEKTISKA
KGQPREPQVYTLPPSQEEMTKNQVSLTCLVKGFYPSDIAV
EWESNGQPENNYKTTPPVLDSDGSFFLYSRLTVDKSRWQE
GNVFSCSVMHEALHNHYTQKSLSLSLGK;
S46-Fc (SEQ ID NO: 128)
EEELQMIQPEKLLLVTVGETATLHCTVTSLLPVGPVLWFR
GVGPGRELIYMSGRGHFPRVTTVSDLTKRENKDFSIRISS
ITPADVGTYYCVKFRKGSPEDVEFKSGPGTEMALGAKPSE
SKYGPPCPPCPAPEFLGGPSVFLFPPKPKDTLMISRTPEV
TCVVVDVSQEDPEVQFNWYVDGVEVHNAKTKPREEQFNST
YRVVSVLTVLHQDWLNGKEYKCKVSNKGLPSSIEKTISKA
KGQPREPQVYTLPPSQEEMTKNQVSLTCLVKGFYPSDIAV
EWESNGQPENNYKTTPPVLDSDGSFFLYSRLTVDKSRWQE
GNVFSCSVMHEALHNHYTQKSLSLSLGK;
S47-Fc (SEQ ID NO: 129)
EEELQMIQPEKLLLVTVGETATLHCTVTSLLPVGPVLWFR
GVGPGRELIYMSGRGHFPRVTTVSDLTKRENKDFSIRISS
ITPADVGTYYCVKFRKGSPEDVEFKSGPGTEVALGAKPSE
SKYGPPCPPCPAPEFLGGPSVFLFPPKPKDTLMISRTPEV
TCVVVDVSQEDPEVQFNWYVDGVEVHNAKTKPREEQFNST
YRVVSVLTVLHQDWLNGKEYKCKVSNKGLPSSIEKTISKA
KGQPREPQVYTLPPSQEEMTKNQVSLTCLVKGFYPSDIAV
EWESNGQPENNYKTTPPVLDSDGSFFLYSRLTVDKSRWQE
GNVFSCSVMHEALHNHYTQKSLSLSLGK;
S48-Fc (SEQ ID NO: 130)
EEELQMIQPEKLLLVTVGETATLHCTVTSLLPVGPVLWFR
GVGPGRELIYRSGRGHFPRVTTVSDLTKRNNKDFSIRISS
ITPADVGTYYCVKFRKGSPEDVEFKSGPGTEMALGAKPSE
SKYGPPCPPCPAPEFLGGPSVFLFPPKPKDTLMISRTPEV
TCVVVDVSQEDPEVQFNWYVDGVEVHNAKTKPREEQFNST
YRVVSVLTVLHQDWLNGKEYKCKVSNKGLPSSIEKTISKA
KGQPREPQVYTLPPSQEEMTKNQVSLTCLVKGFYPSDIAV
EWESNGQPENNYKTTPPVLDSDGSFFLYSRLTVDKSRWQE
GNVFSCSVMHEALHNHYTQKSLSLSLGK;
S49-Fc (SEQ ID NO: 131)
EEELQMIQPEKLLLVTVGETATLHCTVTSLLPVGPVLWFR
GVGPGRELIYRSGRGHFPRVTTVSDLTKRENKDFSIRISS
ITPADVGTYYCVKFRKGSPEDVEFKSGPGTEMALGAKPSE
SKYGPPCPPCPAPEFLGGPSVFLFPPKPKDTLMISRTPEV
TCVVVDVSQEDPEVQFNWYVDGVEVHNAKTKPREEQFNST
YRVVSVLTVLHQDWLNGKEYKCKVSNKGLPSSIEKTISKA
KGQPREPQVYTLPPSQEEMTKNQVSLTCLVKGFYPSDIAV
EWESNGQPENNYKTTPPVLDSDGSFFLYSRLTVDKSRWQE
GNVFSCSVMHEALHNHYTQKSLSLSLGK.

The IgG4 control as a negative control was an antibody against a target related to neither PD-L1 nor CD47. IgG4-Fc and IgG1-Fc comprise only the Fc segment, respectively, and do not comprise any antigen-specific variable region segments.

### TEST EXAMPLES

### Test Example 1. ELISA Experiment of PD-L1-CD47 Bifunctional Fusion Protein Binding to CD47-his Protein

The binding of the PD-L1-CD47 bifunctional fusion protein was detected by measuring the amount of the bifunctional fusion protein bound to the human CD47 or cyno CD47 immobilized on the ELISA plate. CD47-ECD-His (see Table 7) was diluted with PBS to 1 μg/ml and coated on a 96-well ELISA plate. After washing and blocking of the plate, bifunctional fusion protein samples of different concentrations were added, the plate was washed again, and then horseradish peroxidase-goat anti-human (H+L) antibody (Jackson, CAT #109-035-088) was added. The plate was washed again and tetramethyl benzidine solution was added for color development. Finally the stop solution was added, the OD450 was measured on a microplate reader and EC50 values were calculated. The results were shown in Table 8-1 and Table 8-2.

TABLE 7
Sources of CD47 of different germlines

| | MW/ | | | |
|---|---|---|---|---|
| CD47 type | kDa | Cat.No. | Lot.No. | Manufacturer |
| Human CD47 | 15.2 | 12283-H08H | N/A | S.B |
| Cyno CD47 | 15.8 | CD7-C52H1 | 2171b-76VFl- | ACROBiosystems |
| | | | K9 | |

TABLE 8-1
Binding ELISA of PD-L1-CD47 bifunctional fusion protein to human
CD47 and cyno CD47

| | Human CD47-his binding | Cyno CD47-his binding |
|---|---|---|
| Antibody name | EC50 (ng/ml) | EC50 (ng/ml) |
| h1830-S37 | 53.79 | 51.55 |
| h1830-S85 | 71.19 | 66.72 |
| h1830-S19 | 67.35 | 63.43 |
| h1830-S12 | 51.11 | 78.94 |
| h1830-S15 | 64.09 | 72.26 |
| h1831-19-S58 | 46.95 | 59.99 |
| h1830-S58 | 40.79 | 61.79 |
| h1830-19-S79 | 90.53 | 43.53 |
| Hu167 IgG4AA | 70.32 | 31.73 |
| TTI-621 | 30.17 | 293.4 |
| hu5F9 | 37.4 | 30.33 |
| S58-Fc | 24.79 | 15.43 |
| IgG4 | No binding | No binding |

TABLE 8-2
Binding ELISA of PD-L1-CD47 bifunctional fusion protein to human
CD47 and cyno CD47

| | Binding ELISA (EC50, ng/ml) | |
|---|---|---|
| Sample name | Human CD47 | Cyno CD47 |
| h1830-S37 | 40.74 | 30.57 |
| h1830-S85 | 84.66 | 64.08 |
| h1831K-19-S37 | 60.91 | 33.01 |
| S58-Fc | 25.55 | 11.96 |
| S37-Fc | 22.8 | 11.4 |
| IgG4 | No binding | No binding |

The results showed that each PD-L1-CD47 bifunctional fusion protein has a very strong affinity with free human CD47 protein, and also has a very strong cross-affinity with cyno CD47.

### Test Example 2. ELISA Experiment of PD-L1-CD47 Bifunctional Fusion Protein Binding to PD-L1-his Protein

The binding of the PD-L1-CD47 bifunctional fusion protein was detected by measuring the amount of the antibody bound with PD-L1 of different species immobilized on the ELISA plate. PD-L1-his antigen of different germlines (see Table 9) was diluted with PBS to 1 μg/ml and coated on a 96-well ELISA plate (Costar, CAT #3590). After washing and blocking of the plate, PD-L1-CD47 bifunctional fusion protein samples of different concentrations were added, the plate was washed again, and then horseradish peroxidase-goat anti-human (H+L) antibody (Jackson, CAT #109-035-088) was added. The plate was washed again and tetramethyl benzidine solution was added for color development. Finally the stop solution was added, the OD450 was measured on a microplate reader and EC50 values were calculated. The results were shown in Table 10.

TABLE 9
Sources of PD-L1 of different germlines

| | | | | Manufac- |
|---|---|---|---|---|
| PD-LI type | MW/kDa | Cat.No. | Lot.No. | turer |
| hPD-L1-His | 26.8 | 10084-H08H | LC11SE1203 | S.B |
| Cyno PD-L1-His | 26.7 | 90251-C08H | LC10DE1308 | S.B |
| mPD-L1 | 26.3 | 50010-M08H | LC10NO0102 | S.B |

TABLE 10
Binding ELISA of PD-L1-CD47 bifunctional fusion protein
to PD-L1 of different species

| | Human | Mouse | Cyno |
|---|---|---|---|
| | PD-L1-his | PD-L1-his | PD-L1-his |
| | binding | binding | binding |
| | (OD450) | (OD450) | (OD450) |
| | EC50 | EC50 | EC50 |
| Antibody name | (ng/ml) | (ng/ml) | (ng/ml) |
| h1830-S37 | 99.28 | 32 | 176.8 |
| h1830-S85 | 141.8 | 27.48 | 206.6 |
| h1830-S19 | 166.5 | 34.05 | 179.4 |
| h1830-S12 | 146.2 | 34.28 | 178.1 |
| h1830-S15 | 127.8 | 38.63 | 232.1 |
| h1831-19-S58 | 94.68 | No binding | 237.3 |
| h1831-19-S79 | 97.64 | No binding | 214.5 |
| h1830-S58 | 160.6 | 33.52 | 320.1 |
| h1830-19-S79 | 95.16 | 58.2 | 254.1 |
| HRP00052 | 163.1 | No binding | 195.5 |
| h1830 | 131.3 | 28.94 | 230.2 |
| h1831 | 129.2 | No binding | 221.7 |
| h1831K-19-S37 | 46 | No binding | 97.21 |
| IgG4 control | No binding | No binding | No binding |

The results showed that each PD-L1-CD47 bifunctional fusion protein has a very strong affinity with free human PD-L1 protein, and also a very strong cross-affinity with cyno PD-L1. PD-L1-CD47 bifunctional fusion protein comprising h1830 antibody also has a very strong cross-affinity with mouse PD-L1.

### Test Example 3. Blocking Effect of PD-L1-CD47 Bifunctional Fusion Protein on the Binding of PD-L1/PD1 and of PD-L1/B7.1

PD-L1-Fc (prepared in-house) was diluted with PBS to 1 μg/ml, added to a 96-well plate at 100 μl/well, and placed at 4° C. for 16 h-20 h. The PBS buffer was removed from the 96-well plate, which was washed with PBST (pH 7.4 PBS comprising 0.05% tween20) buffer for once. PBST/1% milk was added at 120 μl/well and incubated at room temperature for 1 h for blocking. The blocking solution was removed and the plate was washed with PBST buffer for once. 90 μl of the PD-L1-CD47 bifunctional fusion protein to be tested diluted to appropriate concentrations with sample diluent (pH7.4 PBS comprising 5% BSA, 0.05% Tween20) was added, and pre-incubated at 4° C. for 1 h. 10× concentration of biotin-labeled PD-1 (Beijing Sino Biological Inc., 10 μg/ml) or B7-1 (Beijing Sino Biological Inc., 10 μg/ml) was add at a volume of 10 μl/well. After shaking and mixing on a shaker, the plate was incubated at 37° C. for 1 h. The reaction system was removed and the plate was washed with PBST for 6 times. 100 μl/well Streptavidin-Peroxidase Polymer 1:400 diluted with PBST buffer was added and incubated with shaking at room temperature for 50 min. The plate was washed with PBST for 6 times. 100 μl/well TMB was added and incubated at room temperature for 5-10 min. 100 μl/well 1 M H2504 was added to stop the reaction. OD450 was measured by using NOVOStar on a microplate reader and IC50 value was calculated. The results were shown in Table 11.

TABLE 11
Blocking ELISA of PD-L1-CD47 bifunctional fusion proteins

| | Protein blocking ELISA | |
|---|---|---|
| | Blocking the binding of | Blocking the binding of |
| | human PD-L1-Fc to PD1 | human PD-L1-Fc to B7-1 |
| Antibody name | (OD450) IC50 (ng/ml) | (OD450) IC50 (ng/ml) |
| h1830-S37 | 145.6 | 169.2 |
| h1830-S85 | 81.41 | 148.5 |
| h1830-S19 | 51.83 | 34.23 |
| h1830-S12 | 42.91 | 30.39 |
| h1830-S15 | 47.12 | 55.48 |
| h1831-19-S58 | 122.5 | 159.7 |
| h1831-19-S79 | 72.28 | 113.3 |
| h1830-S58 | 102.5 | 126.8 |
| h1830-19-S79 | 64.69 | 98.84 |
| HRP00052 | 32.04 | 30.71 |
| h1830 | 34.96 | 75.25 |
| h1831 | 26.58 | 49.39 |
| IgG4 control | No binding | No binding |

The test results showed that all bifunctional fusion proteins could also effectively block PD-L1/PD-1 and PD-L1/B7.1 pathways.

### Test Example 4. Experiment of PD-L1-CD47 Bifunctional Fusion Proteins Binding to Human Red Blood Cells

Fresh healthy human blood was mixed with PBS in equal volume and centrifuged at 300 g for 5 min to obtain cell clusters. Red blood cells were obtained after washing with PBS for 3-5 times. Cells were resuspended in FACS buffer (PBS+5% BSA) with the cell density adjusted to 2×10⁶ cells/ml and seeded to a 96-well round bottom plate (3795 #, corning) at 100 μl/well. Then different concentrations of antibodies and bifunctional fusion proteins were added and incubated at 4° C. for 1 hour. After washing twice with FACS buffer (PBS+2% FBS), the secondary antibody (Alexa 488 goat anti-human IgG antibody (Invitrogen, CAT #A11013)) was added and incubated for 30 min on ice in the dark. Finally, the cells were resuspended after washing twice with FACS buffer. The plate was read in FACS Cantoll.

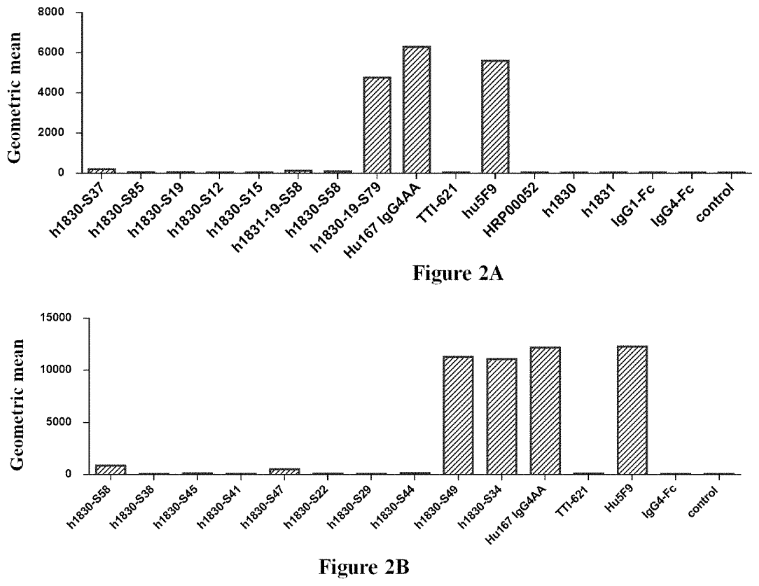

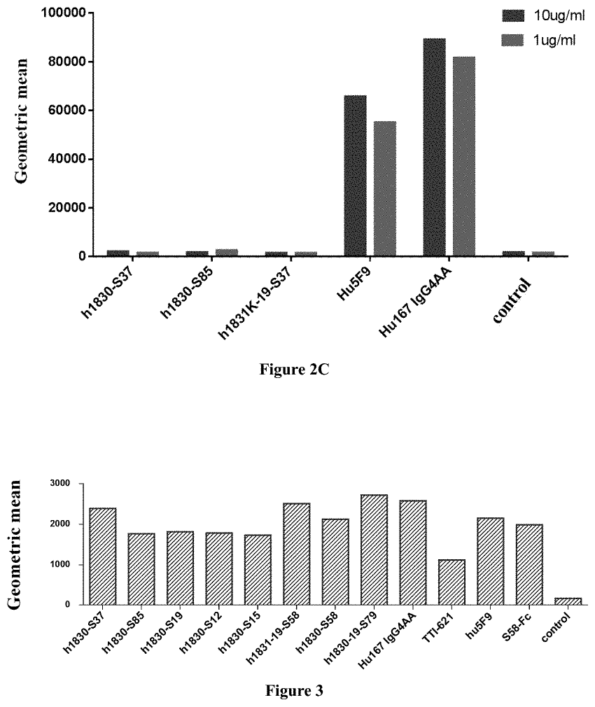

FACS test results showed that the control antibodies hu5F9 and Hu167 IgG4AA have strong binding ability with natural CD47 on the surface of human red blood cells. Among the bifunctional fusion proteins involved, except the bifunctional fusion proteins comprising S79, S34 and S49 which bind with CD47 on the surface of human red blood cells, other bifunctional fusion proteins have no binding ability with natural CD47 on the surface of human red blood cells, suggesting the safety advantages of the above bifunctional fusion proteins. The results were shown in FIG. 2A, FIG. 2B and FIG. 2C (the experiments performed in FIG. 2A, FIG. 2B and FIG. 2C involve three batches of experiments, with different donor cells).

### Test Example 5. Experiment of PD-L1-CD47 Bifunctional Fusion Proteins Binding to Tumor Cells

Raji cells were cultured in RPMI medium (Hyclone, CAT #SH30809.01B) (comprising 10% fetal bovine serum). Raji cells at 1×10⁶ cells/ml were blocked with 5% BSA, the bifunctional fusion protein samples were added to a concentration of 10 μg/ml. After washing twice, Alexa Fluor 488-goat anti-human (H+L) antibody (Invitrogen, CAT #A11013) was added. After washing twice, the fluorescence signal value was read by a flow cytometer.

The FACS test results showed that the PD-L1-CD47 bifunctional fusion proteins involved have a very strong binding ability with the natural CD47 on the surface of Raji cells, which is equivalent to the binding ability of the control antibody Hu5F9. The results were shown in FIG. 3.

### Test Example 6. In Vitro Cell-Mediated Cell Phagocytosis (ADCP) Experiment of PD-L1-CD47 Bifunctional Fusion Proteins

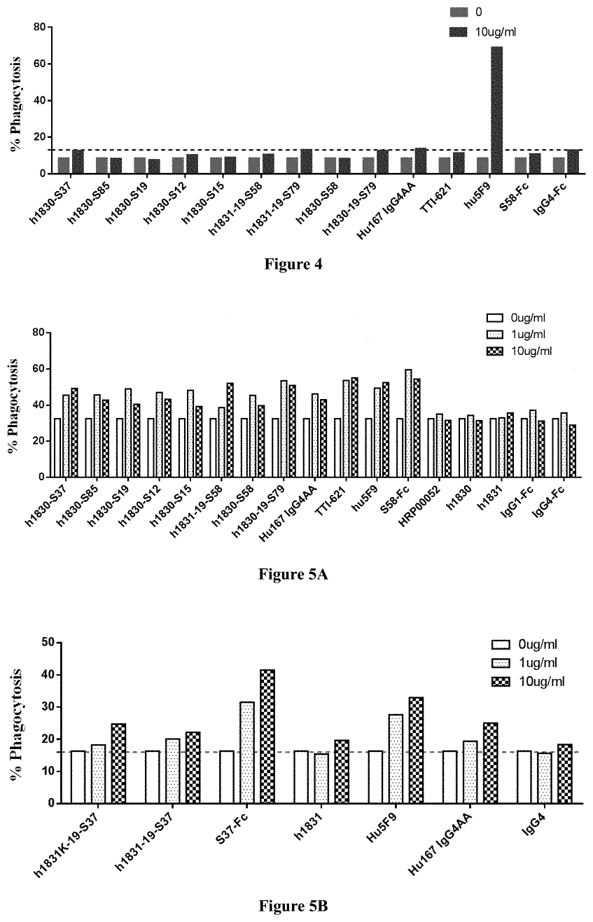

PBMC was isolated from fresh human blood, and then CD14+ monocytes were sorted by using Human CD14 MicroBeads (130-050-201 #, Miltenyi Biotec). These CD14+ monocytes were differentiated into macrophages by culturing in macrophage differentiation medium (1640+10% FBS+50 ng/ml M-CSF) for 9 days. These monocyte-derived macrophages (MDM) became adhesive and had tentacles. On the day of the experiment, the macrophages were digested with trypsin for 5 min, scraped off gently with a scraper and spread on a 96-well round bottom plate (3795 #). Human RBC cells (red blood cells) were labeled with 0.5 μM CFSE (BD, Catalog number 565082 #) in a 37° C. water bath for 13 min. After washing twice with PBS, the samples were added to macrophages at the ratio of 5 RBC cells per each macrophage, and PD-L1-CD47 bifunctional fusion proteins were added at various concentrations. The target cells were subjected to phagocytosis for 2.5 hours. After phagocytosis, cells were washed twice with PBS. Then human Fc blocker (564219 #, BD) was added according to a certain ratio and placed at room temperature for 10 min to exclude non-specific binding. Subsequently, APC-labeled CD14 antibody (17-0149-42 #, Ebioscience) was added. Cells were incubated on ice for 30 min. After the last twice washes, analysis was performed by flow cytometry. Phagocytosis was measured by selecting CFSE+ positive cells in the APC+ positive living cell gate, and then evaluating the percentage of CSFE+ positive cells (see FIG. 4).

### Test Example 7. In Vitro Cell-Mediated Cell Phagocytosis (ADCP) Experiment of PD-L1-CD47 Bifunctional Fusion Proteins

PBMC was isolated from fresh human blood, and then CD14+ monocytes were sorted by using Human CD14 MicroBeads (130-050-201 #, Miltenyi Biotec). These CD14+ monocytes were differentiated into macrophages by culturing in macrophage differentiation medium (1640+10% FBS+50 ng/ml M-CSF) for 9 days. These monocyte-derived macrophages (MDM) became adhesive and had tentacles. On the day of the experiment, the macrophages were digested with trypsin for 5 min, scraped off gently with a scraper and spread on a 96-well round bottom plate (3795 #). Molp-8 cells (Nanjing CoBioer) were labeled with 0.1 μM CFSE in a 37° C. water bath for 13 min. After washing twice with PBS, samples were added to macrophages at the ratio of 5 Molp-8 tumor cells per each macrophage, and PD-L1-CD47 antibodies were added at various concentrations. The target cells were subjected to phagocytosis for 2.5 hours. After phagocytosis, cells were washed twice with PBS. Then human Fc blocker was added according to a certain ratio and placed at room temperature for 10 min to exclude non-specific binding. Subsequently, APC-labeled CD14 antibody was added. Cells were incubated on ice for 30 min. After the last twice washes, analysis was performed by flow cytometry. Phagocytosis was measured by selecting in the APC+ positive living cell gate, and then evaluating the percentage of CSFE+ positive cells (see FIG. 5A and FIG. 5B).

The results of Test Examples 6 and 7 were shown in FIG. 4 and FIG. 5A to FIG. 5B, showing that the added bifunctional fusion proteins could effectively promote the phagocytosis on tumor cells. However, the bifunctional fusion proteins have no phagocytic effect on red blood cells, suggesting the potential advantages of the bifunctional fusion protein antibodies of the present disclosure in terms of safety. Meanwhile, the control antibody hu5F9 could effectively phagocytose red blood cells.

### Test Example 8. Red Blood Cell Agglutination Experiment of PD-L1-CD47 Bifunctional Fusion Proteins

Fresh healthy human blood was diluted 100 times with PBS (B320 #, Shanghai BasalMedia Technologies Co., Ltd.). The diluted whole blood was plated onto a 96-well round bottom plate (3795 #, corning) at 30 μl/well. Then antibodies or bifunctional fusion proteins with different concentration gradients were added in equal volumes. After mixing, the plate was placed at 37° C. for 4-6 h. Sedimentation of red blood cells was observed by using a high-content microscope. Cells with no blood coagulation were clear red spots, and cells with blood coagulation appeared diffused.

Each sample was diluted from the first column (0.5 mg/ml) to the 11th column (1:3 dilution). The 12th column was PBS blank wells without antibody.

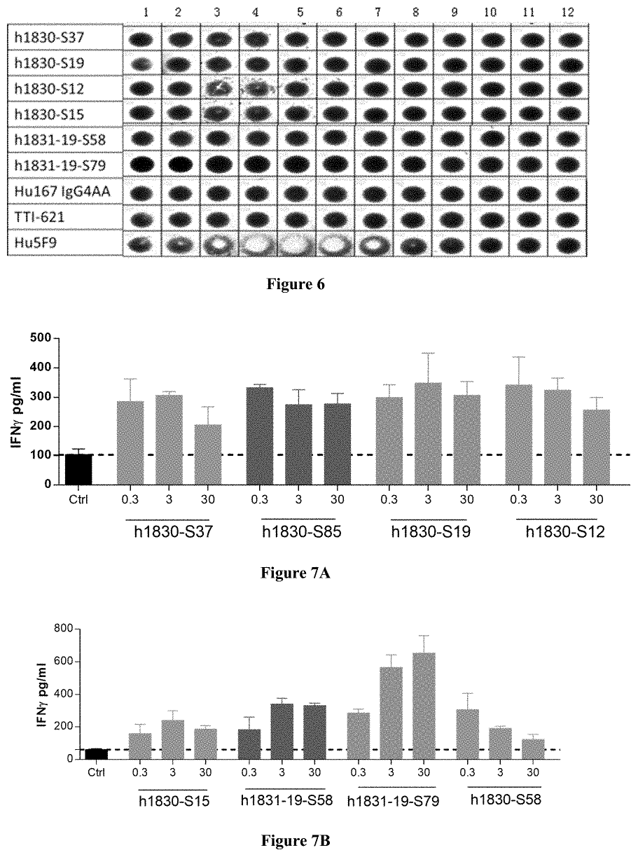

The results showed (see FIG. 6) that under the same conditions, the bifunctional fusion proteins h1830-537, h1830-519, h1830-512, h1830-S15, h1831-19-558 and h1831-19-579 did not cause red blood cell agglutination under the different concentrations tested, suggesting the advantages of the bifunctional fusion proteins of the present disclosure in terms of safety.

### Test Example 9. Affinity Experiment of PD-L1-CD47 Bifunctional Fusion Proteins Detected by BIAcore

The response signals of bifunctional fusion proteins for different antigens were detected in real time by Biacore T200 instrument by using a Protein A biosensor chip (Cat. #29127556, GE) to affinity capture IgG, and different antigens (hCD47, cyno CD47, hPD-L1, cyno PD-L1 and mPD-L1, see Test Examples 3 and 4 for the sources) flowed through the surface of the chip, and the binding and dissociation curves were obtained. Once the dissociation of each experimental cycle was completed, the biosensor chip was washed and regenerated with 10 mM Glycine-HCl pH 1.5 buffer. The experimental buffer system was 1×HBS-EP buffer solution (Cat #BR-1001-88, GE). Once the experiment was completed, the data was fit with (1:1) Langmuir model by using the GE Biacore T200 Evaluation version 3.0 software to obtain the affinity values. The results were shown in Table 12-1 and Table 12-2. The results showed that the affinity of all modified SIRPγ peptide variants to human CD47 was dramatically improved, when compared with that of the wild-type SIRPγ peptide.

TABLE 12-1
Biacore affnity of antibodies to different antigens (KD (M))

| | Human | Cyno | Human | Cyno | Mouse |
|---|---|---|---|---|---|
| | CD47 | CD47 | PD-L1 | PD-Ll | PD-L1 |
| h1830-S37 | 1.72E−09 | 2.03E−09 | 3.87E−10 | 4.09E−10 | 7.09E−08 |
| h1830-S85 | 3.84E−09 | 4.63E−09 | 3.74E−10 | 3.97E−10 | 8.58E−08 |
| h1830-S19 | 5.60E−09 | 6.55E−09 | 3.99E−10 | 4.24E−10 | 6.86E−08 |
| h1830-S12 | 4.36E−09 | 5.11E−09 | 4.00E−10 | 4.29E−10 | 6.98E−08 |
| h1830-S15 | 4.11E−09 | 4.78E−09 | 3.80E−10 | 4.96E−10 | 7.88E−08 |
| h1831-19- | 1.77E−09 | 1.94E−09 | 8.25E−11 | 8.91E−11 | No |
| S58 | | | | | binding |
| h1831-19- | 3.83E−09 | 4.63E−09 | 8.74E−11 | 1.21E−10 | No |
| S79 | | | | | binding |
| h1830-S58 | 1.95E−09 | 2.18E−09 | 3.74E−10 | 3.98E−10 | 7.51E−08 |
| h1830-S79 | 4.06E−09 | 4.90E−09 | 4.25E−10 | 4.55E−10 | 7.75E−08 |
| Hul67 | 6.53E−10 | 1.09E−09 | — | — | — |
| IgG4AA | | | | | |
| TTI-621 | 4.15E−09 | 1.04E−08 | — | — | — |
| Hu5F9 | 5.05E−10 | 8.50E−10 | — | — | — |
| S58-Fc | 5.51E−10 | 9.74E−10 | — | — | — |
| HRP00052 | — | — | 1.44E−10 | 1.51E−10 | No |
| | | | | | binding |
| h1830 | — | — | 4.66E−10 | 4.98E−10 | 9.75E−08 |
| h1831 | — | — | 8.06E−11 | 9.19E−11 | No |
| | | | | | binding |
| h1831-19- | 1.16E−09 | 1.39E−09 | 5.29E−11 | 4.71E−11 | No |
| S37 | | | | | binding |
| h1831K-19- | 1.25E−09 | 1.48E−09 | 5.63E−11 | 4.93E−11 | No |
| S37 | | | | | binding |
| S37-Fc | 5.24E−10 | 8.93E−10 | — | — | — |

TABLE 12-2
Biacore affinity of bifunctional fusion proteins to human
CD47 (KD (M))

| Bifunctional | Biacore (human CD47) |
|---|---|
| fusion protein | KD(M) |
| h1830-S38 | 1.02E−08 |
| h1830-S22 | 6.30E−09 |
| h1830-S29 | 4.59E−09 |
| h1830-S34 | 2.60E−09 |
| h1830-S41 | 5.05E−09 |
| h1830-S42 | 4.19E−09 |
| h1830-S43 | 2.13E−09 |
| h1830-S44 | 2.99E−09 |
| h1830-S45 | 4.85E−09 |
| h1830-S46 | 1.77E−09 |
| h1830-S47 | 1.94E−09 |
| h1830-S48 | 2.49E−09 |
| h1830-S49 | 3.09E−09 |

### Test Example 10. Effect of PD-L1-CD47 Bifunctional Fusion Proteins on the Cell Secretion of IFNγ in PBMC-T Lymphocyte Activation Experiment

In order to study the effect of PD-L1-CD47 bifunctional fusion proteins on the function of human primary T lymphocytes, human peripheral blood mononuclear cells (PBMC) were collected and purified, stimulated with tuberculin (TB) for 5 days in vitro, and the secretion level of the cytokine IFNγ was detected. The experimental process is briefly described as follows:

PBMCs were obtained from fresh blood using Ficoll-Hypaque (17-5442-02, GE) by density gradient centrifugation (Stem Cell Technologies), cultured in RPMI 1640 (SH30809.01, GE) culture medium supplemented with 10% (v/v) FBS (10099-141, Gibco) at 37° C. and 5% CO₂.

The freshly isolated and purified PBMCs were adjusted to a density of 2×10⁶ cells/ml with RPMI 1640 culture medium. 40 μl tuberculin (97-8800, Synbiotics) was added to 20 mL cell suspension and cultured in a 37° C., 5% CO₂ incubator for 5 days. On the 5th day, the aforementioned cultured cells were collected by centrifugation, resuspended in fresh RPMI 1640 culture medium, adjusted to a density of 1.1×10⁶ cells/ml, and seeded into a 96-well cell culture plate at 90 μl per well. At the same time, gradient diluted antibody samples diluted with PBS (B320, Shanghai BasalMedia Technologies Co., Ltd.) were added at 10 μl per well. The cell culture plate was placed in a 37° C., 5% CO₂ incubator and incubated for 3 days. The cell culture plate was taken out and the cell culture supernatant was collected by centrifugation (4000 rpm, 10 min). IFN-γ level was detected by using the ELISA method (human IFN-γ detection kit: EHC102g.96, Neobioscience). Instructions of the reagents were referred to for specific operations.

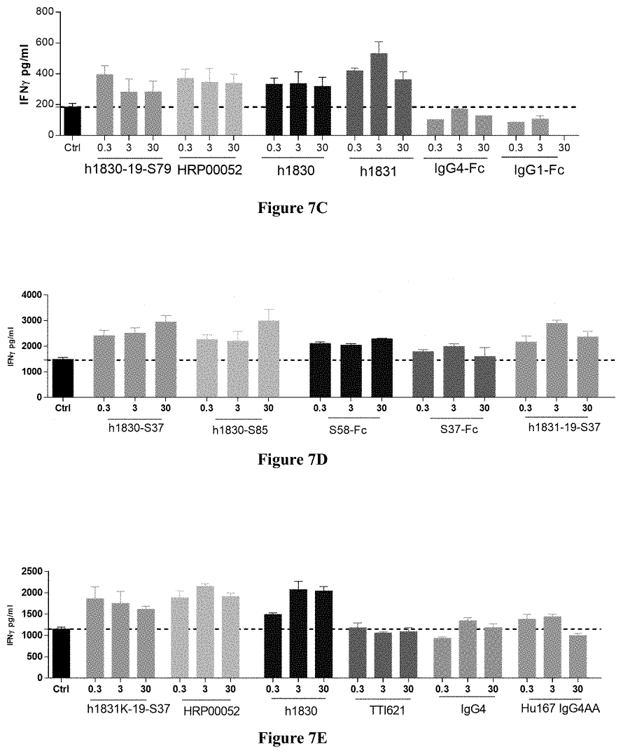

The results (see FIG. 7A to FIG. 7E) showed that among the tested molecules, all bifunctional fusion proteins could activate IFN-γ secretion, which was comparable to the control antibody HRP00052.

### Test Example 11. Effect of PD-L1-CD47 Bifunctional Fusion Proteins in Mouse Colon Cancer Model MC38/H-11-hCD47

In this experiment, B-hCD274/hCD47/hSIRPα mice were used and inoculated with artificially modified murine colon cancer MC38 cells: MC38/H-11-hCD47 (tranfected with human PD-L1 and human CD47, with murine CD47 and PDL1 knocked out) to establish the tumor-bearing mouse model, and the in vivo inhibition effect of different doses of PD-L1-CD47 bifunctional fusion proteins h1830-585, SIRPγ protein S58-Fc and PD-L1 monoclonal antibody h1830 on the growth of murine colon cancer transplanted tumor was evaluated. B-hCD274/hCD47/hSIRPα mice were purchased from Biocytogen Experimental Animals, SPF grade; body weight: 22.0±3.0 g; gender: female.

MC38/H-11-hCD47 (#5-4) cells were inoculated subcutaneously into B-hCD274/hCD47/hSIRPα mice at an inoculum of 1×10⁶ cells/100 μl/mouse. After establishing the tumor-bearing model, the tumor volume was measured and animals with too large and too small body weight and tumor size were excluded. Tumor-bearing mice were randomly divided into 5 groups (n=7) according to tumor volume: PBS control group, h1830-585 high-dose experimental group, h1830-585 low-dose experimental group, h1830 experimental group and S58-Fc experimental group. The date of grouping was set as D0.

1) Tumor growth was observed and recorded by using the method of measuring the tumor diameter. At the same time, the body weight of animals were observed and recorded.

2) The tumor diameter and animal body weight were measured twice a week.

3) On the 17th day after administration, the tumors of animals in the PBS control group were relatively large, thus following the principles of animal welfare, the animals in the PBS experimental group were sacrificed; on the 25th day after administration, the remaining animals in the experimental groups were sacrificed.

4) Tumor volume (TV) calculation formula: TV=½×a×b², wherein a and b represent the long diameter and short diameter of the measured tumor, respectively.

5) Relative tumor growth rate T/C %=(T−T0)/(C−00)×100%

6) Tumor growth inhibition rate TGI %=1−T/C %

Statistical analysis of the experimental data was performed by using Excel and GraphPad. The animal body weight, tumor volume and tumor weight of each group were all presented as mean±standard error (Mean±SEM), and Graphpad Prism 6 software was used for plotting.

This experiment intended to evaluate the inhibitory effect of different doses of PD-L1-CD47 bifunctional fusion proteins on the tumor growth in B-hCD274/hCD47/hSIRPα mouse colon cancer transplanted tumor model. In this experiment, different antibodies or bifunctional fusion proteins were administrated at the same time when grouping.

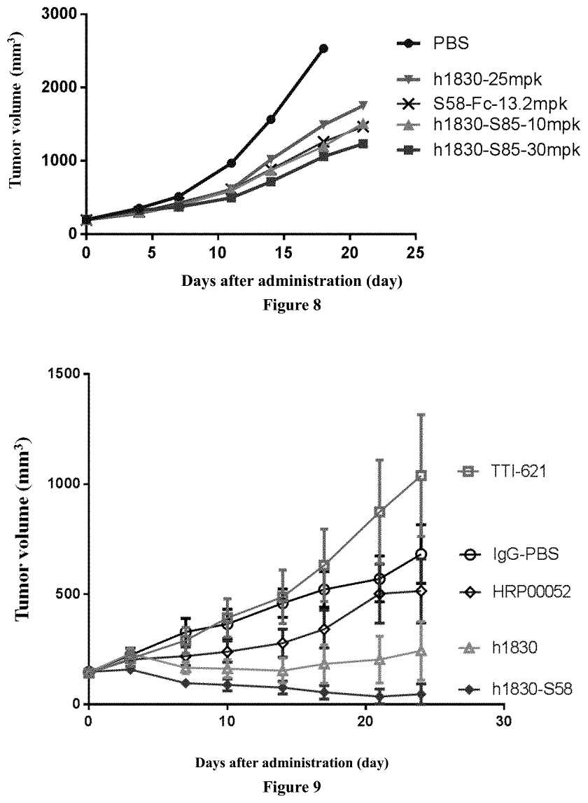

As shown in the results of FIG. 8 and Table 13, different doses of bifunctional fusion protein (h1830-585) experimental group, PD-L1 monoclonal antibody (h1830) experimental group and SIRPγ protein S58-Fc experimental group all have smaller tumor volumes than the PBS control group; the tumor inhibitory effect of the PD-L1-CD47 bifunctional fusion protein high-dose experimental group is better than the same dose of PD-L1 monoclonal antibody experimental group and SIRPγ protein experimental group; there is a dose-dependent relationship among the experimental groups of different h1830-S85 doses.

During the experiment, there was no significant difference in body weight between the administration group and the control group, and the mice were tolerated to each administered antibody.

TABLE 13
Tumor inhibitory effect of antibodies or bifunctional fusion proteins
on transplanted tumors in mice (TGI %)

| | h1830-S85 | h1830-S85 | h1830 | S58-Fc |
|---|---|---|---|---|
| Day | 30 mpk | 10 mpk | 25 mpk | 13.2 mpk |
| 4 | 18.43 | 36.60 | 42.98 | 29.45 |
| 7 | 45.80 | 39.39 | 35.93 | 26.04 |
| 11 | 61.48 | 49.07 | 45.22 | 45.40 |
| 14 | 62.23 | 50.67 | 39.72 | 49.63 |
| 18 | 63.22 | 57.19 | 44.48 | 54.25 |

### Test Example 12. Effect of PD-L1-CD47 Bifunctional Fusion Protein in Mouse MC38-hPD-L1-hCD47 Colon Cancer Model

MC38-hPD-L1-hCD47 cells (an MC38 cells transferred with human PD-L1 and human CD47, but with mouse CD47 knocked out) were inoculated into C57/BL-6 mice subcutaneously at 5.8×10⁵ cells/100 μl/mouse. After establishing the tumor-bearing model, the tumor volume was measured and animals with too large and too small body weight and tumor size were excluded. Tumor-bearing mice were randomly divided into 5 groups (n=7) according to tumor volume: IgG4 control group, h1830-S58 experimental group, HRP00052 experimental group, h1830 experimental group and TTI-621 experimental group. The date of grouping was set as D0. After grouping, each agent was intraperitoneally administered three times a week for a total of 10 times and an administration cycle of 18 days. The tumor-bearing mice were no longer monitored, two days after withdrawal of administration. The tumor volume was measured twice a week, the weight was weighed, and the data was recorded. See the table below for grouping and administration. In this experiment, different antibodies were administrated at the same time when grouping. From the 14th day after administration, the dose of all the experimental groups was reduced to half; from the 25th day after administration, administration was stopped in all experimental groups.

TABLE 14
Experimental grouping and administration

| | Dose of | Route of | Administration |
|---|---|---|---|
| Grouping | agent | administration | cycle |
| IgG-PBS | 10 mpk | i.p. | q.o.d. |
| h1830-S58 | 12 mpk | i.p. | q.o.d |
| HRP00052 | 10 mpk | i.p. | q.o.d |
| h1830 | 10 mpk | i.p. | q.o.d |
| TTI-621 | 5 mpk | i.p. | q.o.d |

Note:
i.p means intraperitoneal injection,
q.o.d means once every other day.

The animal body weight, tumor volume and tumor weight of each group were all presented as mean±standard error (Mean±SEM), and Graphpad Prism 6 and Excel software were used for plotting, and student t test was used for statistical analysis.

Tumor volume (TV)=½×L_{long}×Lₛₕₒᵣₜ²

Tumor growth rate T/C %=(T−T0)/(C−C0)×100%

Tumor growth inhibition rate % TGI=1−T/C %

As shown in the results of FIG. 9, the tumor volume in the PD-L1-CD47 bifunctional fusion protein h1830-S58 experimental group and PD-L1 monoclonal antibody (h1830) experimental group (which cross-reacts with mouse PD-L1) was smaller than that of the control group and TTI-621 experimental group, and the statistical difference compared with the control group was observed around one week after administration; the TTI-621 experimental group did not show tumor inhibitory effect in this experiment. In the h1830-S58 experimental group, 7 days after administration, the tumor inhibition rate reached 128.51%. By the end of the experiment, the tumor inhibition rate remained at a relatively high level.

After the experiment, the tumor-bearing mice were euthanized, and the tumor was collected and weighed. The results for tumor weight were similar to that of the tumor volume. During the experiment, there was no significant difference in body weight between the administration group and the control group, and the mice were tolerated to each administered antibody.

### Test Example 13. Effect of PD-L1-CD47 Bifunctional Fusion Protein in Mouse MC38-hPD-L1 Colon Cancer Model

MC38-hPD-L1 cells (an MC38 cells transferred with human PD-L1) were inoculated into C57/BL-6 mice subcutaneously at 3.5×10⁵ cells/100 μl/mouse. After establishing the tumor-bearing model, the tumor volume was measured and animals with too large and too small body weight and tumor size were excluded. Tumor-bearing mice were randomly divided into 5 groups (n=7) according to tumor volume: IgG4 control group, h1830-19-S79 experimental group, h1830G1-19-S79 experimental group, SIRPα-CV experimental group and h1830 experimental group. The date of grouping was set as D0. After grouping, each agent was intraperitoneally administered three times a week for a total of 12 times and an administration cycle of 28 days. The tumor-bearing mice were no longer monitored, two days after withdrawal of administration. The tumor volume was measured twice a week, the weight was weighed, and the data was recorded. See the table below for grouping and administration.

TABLE 15
Experimental grouping and administration

| | Dose of | Route of | Administration |
|---|---|---|---|
| Grouping | agent | administration | cycle |
| IgG4 control | 10 mpk | i.p. | q.o.d. |
| h1830G1-19-S79 | 12 mpk | i.p. | q.o.d. |
| h1830-19-S79 | 12 mpk | i.p. | q.o.d |
| SIRPa-CV | 5 mpk | i.p. | q.o.d |
| h1830 | 10 mpk | i.p. | q.o.d |

The animal body weight, tumor volume and tumor weight of each group were all presented as mean±standard error (Mean±SEM), and Graphpad Prism 5 and Excel software were used for plotting, and student t test was used for statistical analysis.

Tumor volume (TV)=½×L_{long}×Lₛₕₒᵣₜ²

Tumor growth rate T/C %=(T−T0)/(C−C0)×100%

Tumor growth inhibition rate % TGI=1−T/C %

This experiment intended to evaluate the inhibitory effect of different IgG forms of PD-L1-CD47 bifunctional fusion proteins on the tumor growth in C57/BL-6 mouse colon cancer transplanted tumor model. In this experiment, different antibodies were administrated at the same time when grouping. From the 14th day after administration, the dose in all the experimental groups was reduced to half; from the 25th day after administration, administration was stopped in all experimental groups.

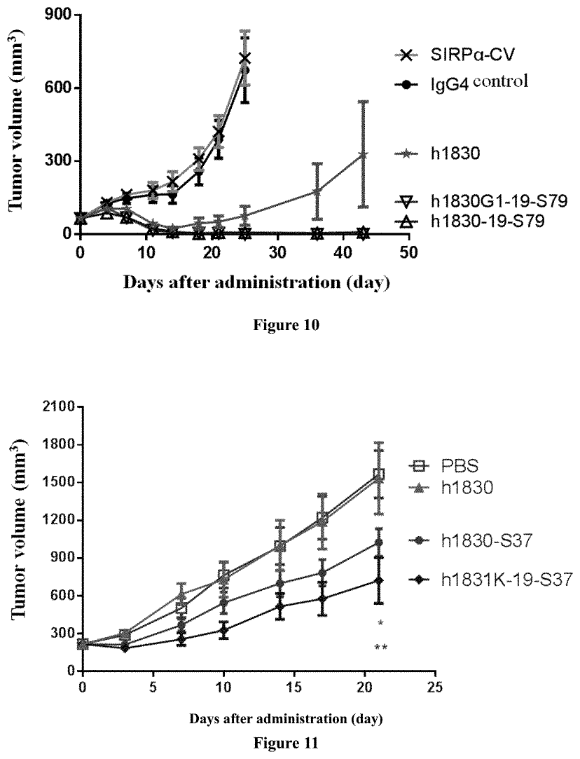

As shown in the results of FIG. 10, until the 25th day after administration, the tumor volume in all the bifunctional fusion protein administration group and PD-L1 monoclonal antibody h1830 administration group was smaller than that of the IgG4 control group and SIRPα-CV (TTI-621) experimental group, and there were statistical differences compared with the control group.

On the 25th day after administration, the control group and the SIRPα-CV (TTI-621) experimental group were euthanized due to the relatively large tumor size, while administration was stopped in the remaining experimental groups and the observation was continued. The results showed that the tumor volume in the PD-L1 monoclonal antibody h1830 experimental group showed a trend of rapid increase over time, and the tumor volume in the bifunctional fusion protein h1830-19-579 and h1830G1-19-579 experimental groups did not change significantly, and there was also no significant difference between these two different IgG forms of bispecific antibodies.

After the experiment, the tumor-bearing mice were euthanized, and the tumor was collected and weighed. The results for tumor weight were similar to that of the tumor volume. During the experiment, there was no significant difference in body weight between the administration group and the control group, and the mice were tolerated to each administered antibody.

### Test Example 14. Efficacy of PD-L1-CD47 Bifunctional Fusion Proteins on MOLP-8 Transplanted Tumor Nude Mice

Balb/c nude mice were inoculated with MOLP-8 cells (5×10⁶+50% matrigel/mouse) subcutaneously at the right ribs for a total of 120 mice. After 10 d, the average tumor volume reached about 214.89±6.75 mm³. The tumor-bearing mice were randomly divided into 7 groups (n=8): PBS control group, h1830-537 experimental group, h1830-S58 experimental group, h1831K-19-S 37, h1830 experimental group, S37-Fc and Hu167 IgG4AA experimental group. The date of grouping was set as D0. After grouping, each agent was intraperitoneally administered twice a week for 3 consecutive weeks. The tumor volume was measured twice a week, the weight was weighed, and the data was recorded. The animal body weight, tumor volume and tumor weight of each group were all presented as mean±standard error (Mean±SEM), and Graphpad Prism 6 and Excel software were used for plotting, and student t test was used for statistical analysis.

Calculation formula of tumor volume (V): V=½×L_{long}=Lₛₕₒᵣₜ²

Relative tumor volume (RTV)=V_{T}/V₀

Tumor growth inhibition rate (%)=(C_{RTV}−T_{RTV})/C_{RTV} (%)

Wherein V₀ and V_{T} were the tumor volume at the start of the experiment and at the end of the experiment, respectively. C_{RTV} and T_{RTV} are the relative tumor volumes of the blank control group (Blank) and the experimental group at the end of the experiment, respectively.

The results of this experiment showed (see FIG. 11) that the mice were intraperitoneally injected once every other day for 10 consecutive administrations. Data until the 21st day of the experiment was used for statistics. The PD-L1-CD47 bifunctional fusion protein h1830-537 (30 mpk) has a tumor inhibition rate of 34.98% (P<0.05); the bifunctional fusion protein h1831K-19-S37 (30 mpk) has a tumor inhibition rate of 54.18% (P<0.01); h1830 (25 mpk) did not have inhibitory effect on tumor growth.

During the administration, the animals in each group had normal body weights, indicating that the bifunctional fusion proteins have no obvious toxicity and side effects.

### Test Example 15. Blocking Effect of PD-L1-CD47 Bifunctional Fusion Proteins on the Binding of CD47/SIRPα

CD47-Fc was diluted with PBS to 1 μg/ml, added to a 96-well plate at 100 μl/well, and placed at 4° C. for 16 h-20 h. The PBS buffer was removed from the 96-well plate, which was washed with PBST (pH 7.4 PBS comprising 0.05% tween20) buffer for once. PBST/1% milk was added at 120 μl/well and incubated at room temperature for 1 h for blocking. The blocking solution was removed and the plate was washed with PBST buffer for once. 90 μl of the PD-L1-CD47 bifunctional fusion protein to be tested diluted to appropriate concentrations with sample diluent (pH 7.4 PBS comprising 5% BSA, 0.05% Tween20) was added and pre-incubated at 4° C. for 1 h. 10× concentration of biotin-labeled SIRPα-his (10 μg/ml) was added at a volume of 10 μl/well, shaken and mixed well on a shaker, and then incubated at 37° C. for 1 h. The reaction system was removed and the plate was washed with PBST for 6 times. 100 μl/well Streptavidin-Peroxidase Polymer 1:400 diluted with PBST buffer was added and incubated with shaking at room temperature for 50 min. The plate was washed with PBST for 6 times. 100 μl/well TMB was added and incubated at room temperature for 5-10 min. 100 μl/well 1 M H₂SO₄ was added to stop the reaction. OD450 was measured by using NOVOStar on a microplate reader and IC50 value was calculated. The results were shown in Table 16.

TABLE 16
Blocking effect of the bispecific antibodies on the
binding of CD47/SIRPα

| Sample | IC50 (ng/ml) |
|---|---|
| h831K-19-S37 | 251.7 |
| S37-Fc | 566 |
| TTI-621 | 25985 |
| Hu5F9 | 263.1 |

The results showed that the bifunctional fusion proteins could effectively block the pathway of CD47 and SIRPα.

### Test Example 16. Efficacy of PD-L1-CD47 Bifunctional Fusion Proteins on Human Breast Cancer Cell MDA-MB-231 Transplanted Tumor

MDA-MB-231 cells (ATCC) were inoculated subcutaneously at the right rib of NOD/SCID mice at 3×10⁶ cells/200 μl/mouse (comprising 50% Matrigel). When the average tumor volume of the tumor-bearing mice reached about 145 mm³, mice were randomly divided into 4 groups: PBS, h1831K-19-S37-30mpk, h1831K-19-S37-10mpk, h1831K-25mpk (maintaining equimolar concentration with that of h1831K-19-S37 high dose) with 8 mice in each group. The grouping day was defined as Day( ) of the experiment. On Day 0, the PBMCs of two volunteers stimulated with CD3 antibody for 3 days were mixed at a ratio of 1:1, and injected into the mouse tumor tissue at 5×10⁵ cells/100 μl/mouse. Stimulation of the remaining PBMCs were stopped, and these PBMCs continued to be cultured for 1 week and then intraperitoneally injected into tumor-bearing mice at 5×10⁶ cells/100 μl/mouse, which was regarded as the first round of injection. By the end of the experiment, a total of two rounds of PBMCs were injected. Starting from Day 0, each antibody to be tested was injected intraperitoneally three times a week. The tumor volume and animal weight were monitored twice a week and the data was recorded. When the tumor volume exceeded 1000 mm³ or ulcer was observed in most tumors or the body weight was reduced by 20%, the tumor-bearing animals were euthanized as the experimental endpoint.

All data were graphed and statistically analyzed by using Excel and GraphPad

Prism 5 software.

Calculation formula of tumor volume (V) is: V=½×a×b², wherein a and b represent length and width respectively.

Relative tumor proliferation rate T/C (%)=(T−T₀)/(C−C₀)×100, wherein T and C are the tumor volume in the treatment group and the control group at the end of the experiment; T₀ and C₀ are the tumor volume at the beginning of the experiment.

Tumor growth inhibition rate (TGI) (%)=1−T/C (%).

The experimental results showed that in the human breast cancer MDA-MB-231 mouse subcutaneously transplanted tumor model, the PDL1-CD47 bispecific antibody h1831K-19-S37 and the PDL1 monoclonal antibody h1831K both showed good tumor inhibition effects (p<0.001 vs PBS).

The PD-L1-CD47 bispecific antibody h1831K-19-S37 (30, 10 mg/kg) could significantly inhibit the growth of human breast cancer MDA-MB-231 mouse subcutaneously transplanted tumors, and there was a dose-dependency between high and low doses. From 3 days after administration till the end of the experiment (Day23), regardless of high-dose group or the low-dose group, the tumor inhibitory effect of h1831K-19-S37 was always better than that of the high-dose PD-L1 monoclonal antibody control h1831K (25 mg/kg) (p<0.001), and there were also statistical differences between high and low doses (p<0.01) (Table 17).

At the endpoint of the experiment, the tumor-bearing mice were euthanized, the tumor was collected and the tumor weight was measured. The results showed that the ex vivo tumor weight was in line with the trend of tumor volume. All treatment groups were significantly better than the control group (p<0.001). Both the high and low dose groups of the bispecific antibody h1831K-19-S37 were better than the high dose PDL1 monoclonal antibody control h1831K (25 mg/kg, p<0.001), and there was a dose-dependent effect between the high and low doses of h1831K-19-S37.

The tumor-bearing mice were tolerated to the PDL1-CD47 bispecific antibody and the monoclonal antibody thereof. There was only a slight fluctuation in body weight during the whole administration process, and no obvious weight loss or other symptoms caused by the agent was observed.

TABLE 17
Inhibitory effect of bispecific antibodies on mice subcutaneous
transplanted tumors

| | Tumor volume | Tumor volume on Day23 | |
|---|---|---|---|
| | on Day0 Mean ± | Mean ± SEM | |
| Group | SEM (mm³) | (mm³) | TGI (%) |
| PBS | 145.8 ± 8.9 | 809.7 ± 41.9 | — |
| hl831K-19-S37-30 mpk | 143.7 ± 10.0 | 73.2 ± 16.1*** | 110.62 |
| h1831K-19-S37-10 mpk | 143.8 ± 8.5 | 163.2 ± 15.4*** | 97.08 |
| hl831K-25 mpk | 144.9 ± 8.4 | 440.4 ± 58.2*** | 55.49 |

Note:
Day0: time of the first administration.
***represents p < 0.001 vs PBS, determined by student’s T test.

1. A bifunctional fusion protein comprising a SIRPγ peptide variant and an anti-human PD-L1 antibody, the SIRPγ peptide variant being linked to the polypeptide chain of the anti-human PD-L1 antibody directly or indirectly through a linker,
wherein the SIRPγ peptide variant is a SIRPγ peptide variant with a substitution mutation at position N51 relative to the wild-type SIRPγ peptide as shown in SEQ ID NO: 20, preferably, the linker is selected from any one of the group consisting of SEQ ID NO: 89-96, (GGGGS)n, (GGGES)n and (GKPGS)n, wherein n is an integer of 2 to 7.

2. The bifunctional fusion protein of claim 1, wherein the carboxyl terminal of the SIRPγ peptide variant is linked to the amino terminal of the heavy chain variable region of the anti-human PD-L1 antibody,
or the carboxyl terminal of the SIRPγ peptide variant is linked to the amino terminal of the light chain variable region of the anti-human PD-L1 antibody,
or the carboxyl terminal of the heavy chain of the anti-human PD-L1 antibody is linked to the amino terminal of the SIRPγ peptide variant,
or the carboxyl terminal of the light chain of the anti-human PD-L1 antibody is linked to the amino terminal of the SIRPγ peptide variant.

3. The bifunctional fusion protein according to claim 1, wherein the SIRPγ peptide variant further comprises amino acid substitution(s) at one or more positions selected from the group consisiting of K19, K53, N101, L31, Q52, E54, H56, N70, M72 and M112 relative to the wild-type SIRPγ peptide.

4. The bifunctional fusion protein of claim 1, wherein the SIRPγ peptide variant with a substitution mutation at position N51 does not substantially bind with CD47 on surface of red blood cells, preferably, the SIRPγ peptide variant with a substitution mutation at position N51 comprises N51F, N51I, N51L, N51M or N51V substitution mutation.

5. The bifunctional fusion protein of claim 1, wherein the SIRPγ peptide variant comprises N51R substitution mutation relative to the wild-type SIRPγ peptide as shown in SEQ ID NO: 20.

6. The bifunctional fusion protein of claim 1, wherein the SIRPγ peptide variant comprises K19E, K53G and N101D substitution mutations relative to the wild-type SIRPγ peptide as shown in SEQ ID NO: 20;
preferably, the SIRPγ peptide variant comprises K19E, N51V, Q52S, K53G, E54R, M72K and N101D mutations relative to the wild-type SIRPγ peptide as shown in SEQ ID NO: 20; or
the SIRPγ peptide variant comprises K19E, N51M, Q52S, K53G, E54R, M72K and N101D mutations relative to the wild-type SIRPγ peptide as shown in SEQ ID NO: 20.

7. The bifunctional fusion protein of claim 6, wherein the SIRPγ peptide variant further comprises amino acid substitution(s) at one or more positions selected from the group consisiting of M6, V27, L30, V33, V36, L37, V42, E47, L66, T67, V92 and S98.

8. The bifunctional fusion protein of claim 6, wherein the SIRPγ peptide variant is as shown in SEQ ID NO: 1.

9. The bifunctional fusion protein of claim 6, wherein the SIRPγ peptide variant is as shown in SEQ ID NO: 2.

10. The bifunctional fusion protein of claim 6, wherein the SIRPγ peptide variant is as shown in any one of the group consisiting of SEQ ID NO: 21, 22, 23, 24, 25, 26, 27, 28, 29, 30, 31, 32, 33, 34, 35, 36, 37, 38, 39 and 40.

11. The bifunctional fusion protein of claim 1, wherein the anti-human PD-L1 antibody is selected from the group consisiting of Avelumab, Atezolizumab, Durvalumab, JS-003, CS-1001, LY-3300054, KD-033, CK-301, CCX-4503, CX-072, KN-035, HRP00052, HRP00049, FAZ-053, GR-1405, KD-005, HLX-20, KL-A167, CBT-502, STI-A1014, REMD-290, BGB-A333, BCD-135 and MCLA-145.

12. The bifunctional fusion protein of claim 1, wherein the anti-human PD-L1 antibody comprises a heavy chain variable region and a light chain variable region, wherein:
the heavy chain variable region comprises HCDR1, HCDR2 and HCDR3 regions with the same sequence(s) as those in the heavy chain variable region as shown in SEQ ID NO: 6, and
the light chain variable region comprises LCDR1, LCDR2 and LCDR3 regions with the same sequence(s) as those in the light chain variable region as shown in SEQ ID NO: 7;
the heavy chain variable region comprises HCDR1, HCDR2 and HCDR3 regions with the same sequence(s) as those in the heavy chain variable region as shown in SEQ ID NO: 8, and
the light chain variable region comprises LCDR1, LCDR2 and LCDR3 regions with the same sequence(s) as those in the light chain variable region as shown in SEQ ID NO: 9; or
the heavy chain variable region comprises HCDR1, HCDR2 and HCDR3 regions with the same sequence(s) as those in the heavy chain variable region as shown in SEQ ID NO: 8, and
the light chain variable region comprises LCDR1, LCDR2 and LCDR3 regions with the same sequence(s) as those in the light chain variable region as shown in SEQ ID NO: 113;
preferably,
the heavy chain variable region comprises HCDR1, HCDR2 and HCDR3 regions as shown in SEQ ID NO: 97, 98 and 99, respectively, and
the light chain variable region comprises LCDR1, LCDR2 and LCDR3 regions as shown in SEQ ID NO: 100, 101 and 102, respectively; or
the heavy chain variable region comprises HCDR1, HCDR2 and HCDR3 regions as shown in SEQ ID NO: 103, 104 and 105, respectively, and
the light chain variable region comprises LCDR1, LCDR2 and LCDR3 regions as shown in SEQ ID NO: 106, 107 and 108, respectively; or
the heavy chain variable region comprises HCDR1, HCDR2 and HCDR3 regions as shown in SEQ ID NO: 103, 104 and 105, respectively, and
the light chain variable region comprises LCDR1, LCDR2 and LCDR3 regions as shown in SEQ ID NO: 106, 112 and 108, respectively.

13. The bifunctional fusion protein of claim 12, wherein the anti-human PD-L1 antibody comprises a heavy chain variable region and a light chain variable region, wherein:
the heavy chain variable region is as shown in SEQ ID NO: 6, and
the light chain variable region is as shown in SEQ ID NO: 7; or
the heavy chain variable region is as shown in SEQ ID NO: 8, and
the light chain variable region is as shown in SEQ ID NO: 113; or
the heavy chain variable region is as shown in SEQ ID NO: 8, and
the light chain variable region is as shown in SEQ ID NO: 9.

14. The bifunctional fusion protein of claim 12, wherein the anti-human PD-L1 antibody further comprises a heavy chain constant region and a light chain constant region, preferably, the heavy chain constant region is as shown in SEQ ID NO: 10 or 11, and the light chain constant region is as shown in SEQ ID NO: 12.

15. The bifunctional fusion protein of claim 14, wherein the anti-human PD-L1 antibody comprises a heavy chain and a light chain, wherein the heavy chain is as shown in SEQ ID NO: 13 or 15, and the light chain is as shown in SEQ ID NO: 14; or
the heavy chain is as shown in SEQ ID NO: 16 or 18, and the light chain is as shown in SEQ ID NO: 17 or 111.

16. The bifunctional fusion protein of claim 15, wherein the bifunctional fusion protein comprises a first polypeptide and a second polypeptide, wherein:
the first polypeptide is selected from the polypeptide as shown in any one of the group consisiting of SEQ ID NO: 41, 42, 43, 44, 45, 46, 47, 48, 49, 50, 51, 52, 53, 54, 55, 56, 57, 58, 59, 60, 61 and 62, and the second polypeptide is selected from the polypeptide as shown in SEQ ID NO: 14; or
the first polypeptide is selected from the polypeptide as shown in any one of the group consisiting of SEQ ID NO: 63, 64, 65, 66, 67, 68, 69, 70, 71, 72, 73, 74, 75, 76, 77, 78, 79, 80, 81, 82 and 109, and the second polypeptide is selected from the polypeptide as shown in SEQ ID NO: 17; or
the first polypeptide is selected from the polypeptide as shown in any one of the group consisiting of SEQ ID NO: 63, 64, 65, 66, 67, 68, 69, 70, 71, 72, 73, 74, 75, 76, 77, 78, 79, 80, 81, 82 and 109, and the second polypeptide is selected from the polypeptide as shown in SEQ ID NO: 111.

17. A SIRPγ peptide variant, wherein the SIRPγ peptide variant is a SIRPγ peptide variant with a substitution mutation at position N51 relative to the wild-type SIRPγ peptide as shown in SEQ ID NO: 20.

18. (canceled)

19. (canceled)

20. (canceled)

21. (canceled)

22. (canceled)

23. (canceled)

24. (canceled)

25. (canceled)

26. (canceled)

27. An anti-human PD-L1 antibody comprising a light chain variable region and a heavy chain variable region, the heavy chain variable region comprises HCDR1, HCDR2 and HCDR3 regions as shown in SEQ ID NO: 103, 104 and 105, respectively, wherein the light chain variable region comprises LCDR1, LCDR2 and LCDR3 regions as shown in SEQ ID NO: 106, 112 and 108, respectively.

28. (canceled)

29. (canceled)

30. (canceled)

31. A pharmaceutical composition comprising a therapeutically effective amount of the bifunctional fusion protein of claim 1, and one or more pharmaceutically acceptable carriers, diluents, buffers or excipients.

32. An isolated nucleic acid molecule encoding the bifunctional fusion protein of claim 1.

33. (canceled)

34. A method for eliminating immunosuppression-related diseases in a subject, which comprises administering to the subject a therapeutically effective amount of the bifunctional fusion protein of claim 1.

35. The method for eliminating immunosuppression-related diseases in a subject of claim 34, wherein the immunosuppression-related diseases include cancer, bacterial or viral infection, preferably, the cancer includes carcinoma, lymphoma, blastoma, sarcoma and leukemia or lymphoid malignancy, more preferably include squamous cell carcinoma, myeloma, small cell lung cancer, non-small cell lung cancer, head and neck squamous cell carcinoma, glioma, Hodgkin's lymphoma, non-Hodgkin's lymphoma, diffuse large B-cell lymphoma, follicular lymphoma, acute lymphoblastic leukemia, acute myelocytic leukemia, chronic lymphocytic leukemia, chronic myelocytic leukemia, primary mediastinal large B-cell lymphoma, mantle cell lymphoma, small lymphocytic lymphoma, T-cell/histiocyte-rich large B-cell lymphoma, multiple myeloma, myeloid cell leukemia-1 protein, myelodysplastic syndrome, gastrointestinal cancer, ovarian cancer, liver cancer, lymphoblastic leukemia, lymphocytic leukemia, colorectal cancer, endometrial cancer, prostate cancer, thyroid cancer, melanoma, chondrosarcoma, neuroblastoma, pancreatic cancer, glioblastoma multiforme, bone cancer, Ewing's sarcoma, cervical cancer, brain cancer, bladder cancer, breast cancer, colon cancer, hepatocellular carcinoma, clear cell renal cell carcinoma, head and neck cancer, pharyngolaryngeal cancer, hepatobiliary cancer, central nervous system cancer, esophageal cancer, malignant pleural mesothelioma, systemic light chain amyloidosis, lymphoplasmacytic lymphoma, myelodysplastic syndrome, myelodysplastic tumor, neuroendocrine tumor, Merkel cell carcinoma, testicular cancer and skin cancer.
